(19) United States
(12) Patent Application Publication
(10) Pub. No.: US 2016/0319347 A1
(43) Pub. Date: Nov. 3, 2016

(54) SYSTEMS AND METHODS FOR DETECTION OF GENOMIC VARIANTS

(71) Applicant: Health Research Inc., Buffalo, NY (US)

(72) Inventors: Carl MOrrison, Fredonia, NY (US); Mary Nesline, Buffalo, NY (US); Jeffrey Conroy, Williamsville, NY (US); Christopher Darlak, Lockport, NY (US)

(73) Assignee: Health Research Inc., Buffalo, NY (US)

(21) Appl. No.: 15/035,063

(22) PCT Filed: Nov. 10, 2014
(86) PCT No.: PCT/US14/64895

§ 371 Date: May 6, 2016

Related U.S. Application Data
(60) Provisional application No. 61/901,890, filed on Nov. 8, 2013.
(60) Provisional application No. 61/936,572, filed on Feb. 6, 2014.
(60) Provisional application No. 61/951,760, filed on Mar. 12, 2014.
(60) Provisional application No. 62/025,845, filed on Jul. 17, 2014.

(51) Int. Cl. C12Q 1/68

(52) U.S. Cl. CPC C12Q 1/6874; C12Q 1/6886; C12Q 2600/156; C12Q 2600/118

(57) ABSTRACT

The invention relates to the detection of genomic variants using next generation sequencing platforms, and increasing the positive predictive value and/or sensitivity of detection. The invention relates to methods and systems for detecting the presence or absence of at least one specific genetic variant, including an allelic variant, in a biological sample. Accurately detecting genetic variants can lead to more accurate diagnosis, prognosis, treatment, and/or prevention of various conditions and disease, including cancer.

### RELATED APPLICATIONS

This application claims benefit of priority to U.S. Provisional Patent Application Ser. No. 61/901,890, filed Nov. 8, 2013; U.S. Provisional Patent Application Ser. No. 61/936,572, filed Feb. 6, 2014; U.S. Provisional Patent Application Ser. No. 61/951,760 filed Mar. 12, 2014; and U.S. Provisional Patent Application Ser. No. 62/025,845, filed Jul. 17, 2014, the contents of which are hereby incorporated by reference in their entireties.

### FIELD OF THE INVENTION

The present disclosure relates to the fields of genomic sequence analysis, diagnostic, prognostic and predictive testing and personalized medicine. In particular, the invention relates to the detection of genomic variants using next generation sequencing platforms, and increasing the positive predictive value and/or sensitivity of detection.

### BACKGROUND

Somatic mutation detection using genomic sequencing of real-life clinical specimens invokes a number of unique challenges. One of the confounding limitations in conventional genomic sequencing assays arises from problems with initially determining the genomic variants present, i.e., mutation or variant calling. When this determination is inaccurate, the positive predictive value (PPV) is low, rendering genomic assays unreliable and leading to inaccurate predictions of patient responsiveness to various therapies. Not only is the reliability of personalized genomic profiles important, but so is the PPV of ascertaining which cancers possess drug-resistant genotypes and/or phenotypes at the time of diagnosis or after diagnosis.

“Variant calling” refers to the process of selecting a nucleotide value, e.g., A, G, T, or C, for a nucleotide position being sequenced. Typically, the sequencing reads (or base calling) for a position will provide more than one value, e.g., some reads will give a T and some will give a G. Variant calling is the process of assigning a nucleotide value, e.g., one of those values to the sequence. Although it is referred to as “variant” calling, it can be applied to assign a nucleotide value to any nucleotide position, e.g., positions corresponding to mutant alleles, wild-type alleles, alleles that have not been characterized as either mutant or wild-type, or to positions not characterized by variability.

Many computational steps are required to translate raw sequencing data output into variant calls. DePristo et al. (2011), Nat. Genet. 43(5):491-98. The sequencing data can be used to detect, for example, single nucleotide polymorphisms (SNPs), multi-nucleotide substitutions, insertions and deletions (indels), microsatellite instability, inversions, fusions, splice variants, isoforms, over-expression, under-expression, translocations, copy number variation, copy neutral loss of heterozygosity, tandem repeats, and/or rearrangements, or any combination thereof.

Identifying true variants from machine errors due to the high rate and context-specific nature of sequencing errors is an outstanding challenge in analyzing sequencing results. For example, the tissue preservative most commonly used, formalin fixation paraffin embedded (FFPE), often leads to variable DNA quality. In addition, some specimens procured for testing (e.g., cancer biopsies) are heterogeneous with varying amounts of normal tissue that leads to additional heterogeneity in the levels of specific variants. As a result, the number of variant reads and the corresponding variant allele frequency (VAF) that defines a given mutation can be difficult to measure. Compounding the issue are the numerous sequencing assays, callers and bioinformatics processes available for variant detection, making it difficult to apply universal methods to identify the “real” somatic variants in a population of numerous false positive variant calls.

In the prior art, the process of analyzing sequencing data can include: initial read mapping; local realignment around indels; base quality score recalibration; SNP discovery and genotyping to find all potential variants; and machine learning to separate true segregating variation from machine artifacts common to next-generation sequencing technologies. Depristo et al. (2011), Nat. Genet. 43, 491-498. The final output of the process is a recalibrated variant call file (VCF). After the VCF is generated, the next process is identifying variants relevant to the patient's disease. Results can indicate not a single concrete call set but instead a continuum from confident to less reliable variant calls. This decreases the usefulness of the variant call, and makes it difficult to perform downstream analysis, including therapeutic and prognostic indications.

### SUMMARY

The present disclosure relates to methods and systems for analyzing genetic sequences to detect variants that are associated with cancer, drug resistance, and other conditions and diseases, and to the use of such detected variants to diagnose, assess, treat, and/or prevent the conditions and diseases.

In one aspect, the present disclosure relates to methods for detecting the presence of at least one specific allelic variant in a biological sample, including receiving first sequencing data produced by sequencing a first aliquot of nucleic acids from the biological sample using a first sequencing platform. The methods also include receiving second sequencing data produced by sequencing a second aliquot of nucleic acids from the biological sample using a second sequencing platform, the first sequencing platform being the same as or differing from the second sequencing platform. In the methods, the first sequencing data and second sequencing data comprise the nucleotide sequences of a multiplicity of sequencing reads including a multiplicity of allelic variants. The methods further include selecting from the multiplicity of allelic variants in the first sequencing data and second sequencing data at least one specific allelic variant for analysis. The presence of the specific allelic variant is detected if a first analysis of the first sequencing data relating to the specific allelic variant passes at least one filter selected from the group consisting of absence of a first platform-dependent systematic error, a first platform-sample-target-dependent minimum variant read threshold and a first platform-sample-target-dependent minimum variant allelic frequency. Alternatively or in conjunction, the presence of the specific allelic variant is detected if a second analysis of the second sequencing data relating to the specific allelic variant passes at least one filter selected from the group consisting of absence of a second platform-dependent systematic error, a second platform-sample-target-dependent minimum variant read threshold and a second platform-sample-target-dependent minimum variant allelic frequency.

In another aspect, the present disclosure relates to methods for detecting the absence of at least one specific allelic variant in a biological sample, including receiving first sequencing data produced by sequencing a first aliquot of nucleic acids from the biological sample using a first sequencing platform. The methods also include receiving second sequencing data produced by sequencing a second aliquot of nucleic acids from the biological sample using a second sequencing platform, the first sequencing platform being the same as or differing from the second sequencing platform. In the methods, the first sequencing data and second sequencing data comprise the nucleotide sequences of a multiplicity of sequencing reads including a multiplicity of allelic variants. The methods further include selecting from the multiplicity of allelic variants in the first sequencing data and second sequencing data at least one specific allelic variant for analysis. The absence of the specific allelic variant is detected if a first analysis of the first sequencing data relating to the specific allelic variant does not pass at least one filter selected from the group consisting of absence of a first platform-dependent systematic error, a first platform-sample-target-dependent minimum variant read threshold and a first platform-sample-target-dependent minimum variant allelic frequency. Alternatively or in conjunction, the absence of the specific allelic variant is detected if a second analysis of the second sequencing data relating to the specific allelic variant does not pass at least one filter selected from the group consisting of absence of a second platform-dependent systematic error, a second platform-sample-target-dependent minimum variant read threshold and a second platform-sample-target-dependent minimum variant allelic frequency.

In another aspect, the present disclosure relates to a method including receiving first sequencing data indicative of a presence or absence of a specific allelic variant in a biological sample based on results from a first sequencing process performed on a first sequencing platform. The first sequencing data comprises nucleotide sequences of a multiplicity of sequencing reads including a first multiplicity of allelic variants. The method also includes receiving second sequencing data indicative of a presence or absence of the specific allelic variant in the biological sample based on results from a second sequencing process performed on a second sequencing platform. The second sequencing data comprises nucleotide sequences of a multiplicity of sequencing reads including a second multiplicity of allelic variants. The method further includes determining at least one first filter value based on base-pair level characteristics of a biological standard comprising the specific allelic variant detected by the first sequencing platform, wherein the at least one first filter value is selected from the group consisting of: a first platform-sample-target-dependent minimum variant reads threshold, a first platform-sample-target-dependent minimum variant allelic frequency, and a first sample-dependent set of systematic errors. The method also includes conducting a first comparison of the at least one first filter value to the first sequencing data to determine if the data indicative of the presence or absence of the specific allelic variant passes the first filter value and determining at least one second filter value based on base-pair level characteristics of the biological standard comprising the specific allelic variant detected by the second sequencing platform, wherein the at least one second filter value is selected from the group consisting of: a second platform-sample-target-dependent minimum variant reads threshold, a second platform-sample-target-dependent minimum variant allelic frequency, and a set sample-dependent of second systematic errors. The method also includes conducting a second comparison of the at least one second filter value to the second sequencing data to determine if the data indicative of the presence or absence of the specific allelic variant passes the second filter value and detecting the presence or absence of the specific allelic variant in the biological sample based on the results of the first comparison and the second comparison.

In a further aspect, the present disclosure relates to a system that includes a first sequencing platform apparatus, a second sequencing platform apparatus, and a multi-platform variant detection system. The multi-platform variant detection system includes a first interface for receiving first sequencing data indicative of a presence or absence of a specific allelic variant in a biological sample based on results from a first sequencing process performed on the first sequencing platform, a second interface for receiving second sequencing data indicative of a presence or absence of a specific allelic variant in the biological sample based on results from a second sequencing process performed on the second sequencing platform, and a computer-readable memory. The computer-readable memory comprises at least one first filter value based on base-pair level characteristics of a biological standard comprising the specific allelic variant detected by the first sequencing platform. The first filter value is selected from the group consisting of: a first platform-sample-target-dependent minimum variant reads threshold, a first platform-sample-target-dependent minimum variant allelic frequency, and a first sample-dependent set of systematic errors. The computer-readable memory also comprises at least one second filter value based on base-pair level characteristics of the biological standard comprising the specific allelic variant detected by the second sequencing platform. The second filter value is selected from the group consisting of: a second platform-sample-target-dependent minimum variant reads threshold, a second platform-sample-target-dependent minimum variant allelic frequency filter, and a second sample-dependent set of systematic errors. The computer-readable memory further comprises instructions that when executed cause the multi-platform variant detection system to: conduct a first comparison of the first at least one filter value to the first sequencing data to determine if the data indicative of the presence or absence of the specific allelic variant passes the at least one first filter value, conduct a second comparison of the second at least one filter value to the second sequencing data to determine if the data indicative of the presence or absence of the specific allelic variant passes the second at least one filter value, and detect the presence or absence of the specific allelic variant in the biological sample based on the results of the first comparison and the second comparison.

In some embodiments, in the disclosed systems and methods, the first sequencing data indicative of the presence or absence of a specific allelic variant in the biological sample is based on sequencing nucleic acids amplified from the biological sample using the first sequencing platform.

In some embodiments, in the disclosed systems and methods, the second sequencing data indicative of the presence or absence of a specific allelic variant in the biological sample is based on sequencing nucleic acids amplified from the biological sample using the second sequencing platform.

In some embodiments, in the disclosed systems and methods, the specific allelic variant is selected from a subset of the multiplicity of variants comprising known therapeutically actionable variants.

In some embodiments, in the disclosed systems and methods, the specific allelic variant is selected from a subset of the multiplicity of variants which does not include at least one known therapeutically non-actionable variant.

In some embodiments, in the disclosed systems and methods, the specific allelic variant is selected from a subset of possible variants which comprises known diagnostically informative variants.

In some embodiments, in the disclosed systems and methods, the specific allelic variant is selected from a pre-defined list of variants which does not include at least one known diagnostically non-informative variant.

In some embodiments, in the disclosed systems and methods, the specific allelic variant is selected from a subset of possible variants which comprises known prognostically informative variants.

In some embodiments, in the disclosed systems and methods, the specific allelic variant is selected from a subset of possible variants which does not include at least one known prognostically non-informative variant.

In some embodiments, in the disclosed systems and methods, the at least one first filter value in the first comparison is the first platform-sample-target-dependent minimum variant read threshold.

In some embodiments, in the disclosed systems and methods, the at least one first filter value in the second comparison is the second platform-sample-target-dependent minimum variant read threshold.

In some embodiments, in the disclosed systems and methods, at least one of the first and the second platform-sample-target-dependent minimum variant read threshold is empirically determined by sequencing at least one control nucleic acid sample.

In some embodiments, in the disclosed systems and methods, at least one of the first and the second platform-sample-target-dependent minimum variant read threshold is known from sequencing at least one control nucleic acid sample.

In some embodiments, in the disclosed systems and methods, the control nucleic acid sample comprises the specific allelic variant.

In some embodiments, in the disclosed systems and methods, the at least one first filter value in the first comparison is the first platform-sample-target-dependent minimum variant allele frequency.

In some embodiments, in the disclosed systems and methods, the at least one filter value in the second comparison is the second platform-sample-target-dependent minimum variant allele frequency.

In some embodiments, in the disclosed systems and methods, at least one of the first and the second platform-sample-target-dependent minimum variant allele frequency is empirically determined by sequencing at least one control nucleic acid sample.

In some embodiments, in the disclosed systems and methods, at least one of the first and the second platform-sample-target-dependent minimum variant allele frequency is known from sequencing at least one control nucleic acid sample.

In some embodiments, in the disclosed systems and methods, the control nucleic acid sample comprises the specific allelic variant.

In some embodiments, in the disclosed systems and methods, at least one of the first and the second platform-sample-target-dependent minimum variant allele frequency is less than 4.0%.

In some embodiments, in the disclosed systems and methods, at least one of the first and the second platform-sample-target-dependent minimum variant allele frequency is less than 3.5%.

In some embodiments, in the disclosed systems and methods, at least one of the first and the second platform-sample-target-dependent minimum variant allele frequency is less than 3.0%.

In some embodiments, in the disclosed systems and methods, at least one of the first and the second platform-sample-target-dependent minimum variant allele frequency is less than 2.5%.

In some embodiments, in the disclosed systems and methods, at least one of the first and the second platform-sample-target-dependent minimum variant allele frequency is less than 2.0%.

In some embodiments, in the disclosed systems and methods, the at least one filter value in the first comparison is absence of at least one first sample-dependent systematic error.

In some embodiments, in the disclosed systems and methods, the at least one filter value in the second comparison is absence of at least one second first sample-dependent systematic error.

In some embodiments, in the disclosed systems and methods, at least one of the first and the second sample-dependent systematic error is empirically determined by sequencing at least one control nucleic acid sample comprising the specific allelic variant.

In some embodiments, in the disclosed systems and methods, at least one of the first and the second sample-dependent systematic error is known from sequencing at least one control nucleic acid sample.

In some embodiments, in the disclosed systems and methods, the control nucleic acid sample comprises the specific allelic variant.

In some embodiments, in the disclosed systems and methods, the detecting the presence of the specific allelic variant further requires that either: (i) the first comparison of the first sequencing data relating to the specific allelic variant passes at least two filters values selected from the group consisting of the first platform-sample-target-dependent minimum variant reads threshold, the first platform-sample-target-dependent minimum variant allelic frequency, and absence of the first sample-dependent set of systematic errors, or (ii) the second comparison of the second sequencing data relating to the specific allelic variant passes at least two filters values selected from the group consisting of the second platform-sample-target-dependent minimum variant reads threshold, the second platform-sample-target-dependent minimum variant allelic frequency, and absence of the second sample-dependent set of systematic errors.

In some embodiments, in the disclosed systems and methods, detecting the presence of the specific allelic variant further requires that either: (i) the first comparison of the first sequencing data relating to the specific allelic variant passes at least three filters values selected from the group consisting of the first platform-sample-target-dependent minimum variant reads threshold, the first platform-sample-target-dependent minimum variant allelic frequency, and absence of the first sample-dependent set of systematic errors, or (ii) the second comparison of the second sequencing data relating to the specific allelic variant passes at least three filters values selected from the group consisting of the second platform-sample-target-dependent minimum variant reads threshold, the second platform-sample-target-dependent minimum variant allelic frequency, and absence of the second sample-dependent set of systematic errors.

In some embodiments, in the disclosed systems and methods, the first sequencing data produced by the first sequencing platform includes a call that the specific allelic variant is present but the second sequencing data produced by the second sequencing platform does not include a call that the specific allelic variant is present.

In some embodiments, in the disclosed methods, the conducting the first comparison includes forming a first subset of sequencing data including only those values from the first sequencing data that do not exhibit the presence of the first sample-dependent set of systematic errors and conducting a further comparison of the first subset of sequencing data to at least one of the first platform-sample-target-dependent minimum variant reads threshold and the first platform-sample-target-dependent minimum variant allelic frequency to determine if the data indicative of the presence or absence of the specific allelic variant in the first subset passes the at least one of the first platform-sample-target-dependent minimum variant reads threshold and the first platform-sample-target-dependent minimum variant allelic frequency.

In some embodiments, in the disclosed systems, the computer-readable instructions that cause the multi-platform variant detection system to conduct the first comparison includes instructions that cause the multi-platform variant detection system to: form a first subset of sequencing data including only those values from the first sequencing data that do not exhibit the presence of the first sample-dependent set of systematic errors, and conduct a further comparison of the first subset of sequencing data to at least one of the first platform-sample-target-dependent minimum variant reads threshold and the first platform-sample-target-dependent minimum variant allelic frequency to determine if the data indicative of the presence or absence of the specific allelic variant in the first subset passes the at least one of the first platform-sample-target-dependent minimum variant reads threshold and the first platform-sample-target-dependent minimum variant allelic frequency.

In some embodiments, in the disclosed methods, the conducting the second comparison includes: forming a second subset of sequencing data including only those values from the second sequencing data that do not exhibit the presence of the second sample-dependent set of systematic errors, and conducting a further comparison of the second subset of sequencing data to at least one of the second platform-sample-target-dependent minimum variant reads threshold and the second platform-sample-target-dependent minimum variant allelic frequency to determine if the data indicative of the presence or absence of the specific allelic variant in the second subset passes the at least one of the second platform-sample-target-dependent minimum variant reads threshold and the second platform-sample-target-dependent minimum variant allelic frequency.

In some embodiments, in the disclosed systems, the computer-readable instructions that cause the multi-platform variant detection system to conduct the second comparison includes instructions that cause the multi-platform variant detection system to: form a second subset of sequencing data including only those values from the second sequencing data that do not exhibit the presence of the second sample-dependent set of systematic errors, and conduct a further comparison of the second subset of sequencing data to at least one of the second platform-sample-target-dependent minimum variant reads threshold and the second platform-sample-target-dependent minimum variant allelic frequency to determine if the data indicative of the presence or absence of the specific allelic variant in the second subset passes the at least one of the second platform-sample-target-dependent minimum variant reads threshold and the second platform-sample-target-dependent minimum variant allelic frequency.

In some embodiments, in the disclosed systems and methods, the first sequencing platform apparatus differs from the second sequencing platform apparatus.

In some embodiments, in the disclosed systems and methods, the first sequencing platform apparatus is the same as the second sequencing platform apparatus.

In some embodiments, in the disclosed systems and methods, the first sequencing platform apparatus includes an Illumina MiSeg™ sequencer apparatus and the second sequencing platform apparatus includes an Ion PGM™ sequencer apparatus.

These and other aspects and embodiments of the disclosure are illustrated and described below. Other systems, processes, and features will become apparent to one with skill in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

FIG. 1A-B is a schematic diagram of prior art variant detection methods using a single sequencing platform.

FIG. 2A-B is a schematic diagram indicating showing some embodiments of the disclosed multi-platform variant detection (MPVD) methods and systems.

FIG. 3A-J shows a comparison of the results of sequencing 41 biological samples from patients using (1) a first sequencing platform alone (MiSeq™ or Illumina) (FIGS. 3A, 3C, 3F, and 3H); (2) the first sequencing platform with the platform-sample-target-dependent MVRT and MVAF values of the invention (FIGS. 3B, 3D, 3G, and 3I) and (3) both a first sequencing platform and a second sequencing platform using the platform-sample-target-dependent MVRT and MVAF values and variant detection methods of the invention (FIGS. 3E and 3J).

FIG. 4 is a schematic diagram detailing the 20-gene validation workflow that exemplifies embodiments of the disclosed multi-platform variant detection (MPVD) methods and systems.

FIG. 5A-D are graphical representations of calculations for a specific filter—a minimum variant reads threshold (MVRT)—used in embodiments of the disclosed methods and systems.

FIG. 6A-D are graphical representations showing that a quality (QUAL) score of 100 for single nucleotide variants (SNVs) for PGM FF, PGM FFPE, MiSeq FF, and MiSeq FFPE resulted in a substantial decrease in false positives while minimally impacting true positives.

FIG. 7A-D are graphical representations showing that a QUALT of 100 for indel variant calls for PGM FF, PGM FFPE, MiSeq FF, and MiSeq FFPE decreased false positives with no impact to true positives, similar to the results for SNV(s).

FIG. 8 is a graphical representation of the numbers of unique SNV systematic errors in the 20-gene validation testing.

FIG. 9 is a schematic showing total and unique systematic errors in two sequencing platforms in the 20-gene validation testing.

FIG. 10A-D are graphical representations showing minimum variant allelic frequency (MVAF) for each sequencing platform and tissue fixation type. The lowest VAF for analytical sensitivity was achieved with MiSeq FF (1.7% VAF). The value for PGM FFPE at 1.8% VAF was similar and with only minor differences to PGM FF (2.9% VAF) and MiSeq FFPE (3.6% VAF).

FIG. 11A-D are graphical representations of analytical positive predictive value (PPV) for single nucleotide variants in the 20-gene validation testing.

FIG. 12 is a summary of results using the 20-gene validation testing, which exemplifies embodiments of the disclosed methods and systems.

### DETAILED DESCRIPTION

The present disclosure relates to methods and systems for analyzing genetic sequencing data to accurately call the presence or absence of sequence variants. The presence of these sequence variants can be associated with conditions and diseases such as cancer and drug response or resistance. Accurate variant calling helps identify the proper diagnosis, prognosis, and/or treatment for patients with particular conditions and disorders. For example, identifying an actionable variant helps an oncologist determine the appropriate patient-specific therapeutic indications for patients with cancers that are susceptible or resistant to specific treatments. In other embodiments, determining the absence of an allelic variant helps reduce unnecessary treatment and ensure proper diagnosis and prognosis of patients. Using prior art single platform analysis can result in variant calls that are discordant depending on the platform chosen. In some embodiments, the multi-platform variant detection methods and systems described herein resolve discordant calls by using two or more platforms. In some embodiments, the invention can be used in connection with the treatment of patients with cancer, including drug-resistant, metastatic, solid and circulating tumors, and with classifying individuals based on characteristics such as drug responsiveness, side effects, and optimal drug dose.

In some aspects, the disclosed methods and systems reduce false negative and/or false positive variant calls by analyzing sequencing data produced by two different sequencing platforms, and applying to each set of sequencing data at least one filter, such as absence of a platform-dependent systematic error, a first platform-sample-target-dependent minimum variant read threshold, and/or a first platform-sample-target-dependent minimum variant allelic frequency. By using at least two sequencing platforms, and analyzing the sequencing data using the filters described herein, the present disclosure increases the accuracy of diagnosis, prognosis, and therapeutic regimes for various conditions, such as cancer, as compared to using current sequencing platforms and methods.

### DEFINITIONS

In order that the present disclosure may be more readily understood, certain terms used in the disclosure and appended claims are specifically defined below. Additional definitions are set forth throughout the detailed description.

As used in this specification and the appended claims, the singular forms “a”, “an” and “the” include plural referents unless the content clearly dictates otherwise. For example, reference to “a nucleic acid” includes a combination of two or more nucleic acids, and the like. As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the term “next generation sequencing” or “NGS” refers to high-throughput sequencing of large numbers of nucleic acids (e.g., genomic DNA, cDNA) in parallel. Examples include, but are not limited to single-molecule real-time sequencing (SMRT™, Pacific Biosciences, Menlo Park, Calif.), ion semiconductor sequencing (Ion PGM™, and Ion Proton™, Life Technologies Corp., Logan, Utah), pyrosequencing (454 Life Sciences, Roche Diagnostics Corp., Basel, Switzerland), sequencing by synthesis (HiSeg™ and MiSeg™, Illumina, Inc., San Diego, Calif.), sequencing by ligation (SOLiD™, Life Technologies Corp. Logan, Utah), nanopore sequencing, tunneling currents sequencing, sequencing by hybridization, mass spectrometry sequencing, microfluidic Sanger sequencing, RNA polymerase (RNAP) sequencing and others.

As used herein, the term “sequencing platform” refers to a system for sequencing nucleic acids, including genomic DNA (gDNA), complementary DNA (cDNA) and RNA. The system may include one or more machines or apparatuses (e.g., amplification machines, sequencing machines, detection devices, etc.), data storage and analytical devices (e.g., hard drives, remote storage systems, processors, etc.), reagents (e.g., primers, probes, linkers, tags, NTPs, etc.) and particular methods for their use. For example, sequencing by synthesis and pyrosequencing and different platforms. However, the same machine may be used in various ways (e.g., with different reagents) and, therefore, represent two different platforms (e.g., use of a single next generation sequencing platform with different methods of amplification of the sample).

As used herein, the term “read” refers to a single instance of determining the identity of a nucleotide at a particular position or the sequence of nucleotides in a particular polynucleotide. If a nucleotide or polynucleotide sequence is determined X times in a sequencing assay, there are “X reads” or a “read depth of X” or “read coverage of X” for that nucleotide or polynucleotide.

As used herein, the term “SAM file” refers to a “Sequence Alignment/MAP file,” a tab-delimited data file for representing genetic sequences, alignments of sequences and variants of sequences. The SAM format has been developed by the SAM/BAM Format Specification Working Group. See Li et al. (2009), Bioinformatics, 25:2078-9.

As used herein, the term “BAM file” refers to the binary equivalent of a SAM file. The BAM format has been developed by the SAM/BAM Format Specification Working Group. BAM files are typically produced by NGS sequencing platforms to represent the raw results of a sequencing assay

As used herein, the term “Variant Call Format file” or “VCF file” refers to a text file for representing genetic sequence variants and associated bioinformatics and sequencing information. VCF files are typically produced by NGS sequencing platforms to summarize the results of a sequencing assay.

As used herein, the term “true positive” and the abbreviation “TP” refer to a positive result from a test or assay (e.g., indicating the presence of an analyte or fulfillment of a condition) which corresponds to an actual positive (e.g., the presence of the analyte or fulfillment of the condition). True positives are positive results which are correctly identified and factually correct. In some embodiments of the invention, a true positive indicates that a genetic sequencing test of a biological sample (e.g., a tumor biopsy) indicates the correct identification of a particular variant allele (e.g., an oncogenic allele or tumor marker), and the biological sample does, in fact, include the variant allele.

As used herein, the term “false positive” and the abbreviation “FP” refer to a positive result from a test or assay (e.g., indicating the presence of an analyte or fulfillment of a condition) which corresponds to an actual negative (e.g., the absence of the analyte or non-fulfillment of the condition). False positives are positive results which are incorrectly identified and factually incorrect.

As used herein, the term “true negative” and the abbreviation “TN” refer to a negative result from a test or assay (e.g., indicating the absence of an analyte or non-fulfillment of a condition) which corresponds to an actual negative (e.g., the absence of the analyte or non-fulfillment of the condition). True negatives are negative results which are correctly identified and factually correct.

As used herein, the term “false negative” and the abbreviation “FN” refer to a negative result from a test or assay (e.g., indicating the absence of an analyte or non-fulfillment of a condition) which corresponds to an actual positive (e.g., the presence of the analyte or fulfillment of the condition). False negatives are negative results which are incorrectly identified and factually incorrect.

As used herein, the term “positive predictive value (PPV)” relates to the precision of test or assay. Mathematically, the PPV is the number of true positives divided by the sum of true positives plus false positives (Tops/Tops+Fops). In some embodiments, PPV is calculated at the assay level (“assay PPV”) or at the sample level (“sample PPV”). In some embodiments, the PPV is calculated for a given variant type (or a collection of variant types) on a specific sequencing platform. In some embodiments, the PPV is calculated at a fixed variant allelic frequency that is chosen to reflect the actual clinical scenario.

As used herein, the term “sensitivity” refers to the true positive rate. Mathematically, it is the number of true positives divided by the number of true positives plus false negatives (TPs/TPs+FNs). In some embodiments, sensitivity is calculated at the assay level (“assay sensitivity”) or the sample level (“sample sensitivity”). In some embodiments, the assay sensitivity is calculated for a given variant type (or a collection of variant types) on a specific sequencing platform. In some embodiments, the assay sensitivity is calculated at a fixed variant allelic frequency in the test samples that are chosen to reflect the actual clinical scenario.

As used herein, the term “minimum variant reads” means the minimum number of variant reads at any given number of total reads for which there is a particular confidence (such as 95% confidence) that a particular percentage (such as 95%) of all variants are detected in a sample containing variants with a majority of VAFs near the assay's sensitivity. The “minimum variant read” used in a filter may be referred to herein as the “minimum variant read threshold” or “MVRT.”

As used herein, the term “empirical minimum variant reads” means the minimum variant reads at any given number of total reads, for a particular confidence level and for a particular level of sensitivity, which is empirically determined by testing a representative reference sample (e.g., a control sample with known levels of variants, or a sample which is tested repeatedly to refine the determination of allelic variants).

As used herein, the term “minimum percent variant reads” means the proportion of variant reads in a background of normal reads required to call a variant “detected” at particular confidence and sensitivity parameters.

As used herein, the term “empirical minimum percent variant reads” means the minimum percent variant reads in a background of normal reads required to call a variant “detected” at particular confidence and sensitivity parameters, which is empirically determined by testing a representative reference sample (e.g., a control sample with known levels of variants, or a sample which is tested repeatedly to refine the determination of allelic variants).

As used herein, the term “quality (QUAL) score” means a Phred-scaled quality score assigned by a variant detector or determined from a BAM or equivalent file. Higher QUAL scores indicate higher confidence in the variant calling and lower probability of errors. For a quality score of Q, the estimated probability of an error is 10^{-(Q/10)}. For example, a set of Q20 calls has a 1% error rate, and a set of Q30 calls has a 0.1% error rate.

As used herein, the term “minimum variant allelic frequency” means the variant allelic frequency for which a particular sensitivity (for example, at least 95% sensitivity) is obtained at any level of coverage. Coverage is the number of times that a given nucleotide in the sequence has been read or sequenced. An allele frequency at a locus is the number of copies of a particular allele divided by the total number of copies of all alleles at that locus. The MVAF is an equivalent measure of analytical sensitivity for all variants in a given sample.

As used herein, the term “empirical minimum variant allele frequency” means the minimum variant allele frequency at any given number of total reads, for a particular confidence level and for a particular level of sensitivity, which is empirically determined by testing a representative reference sample (e.g., a control sample with known levels of variants, or a sample which is tested repeatedly to refine the determination of allelic variants).

As used herein, the term “systematic error” refers to errors at some genomic positions that appear with greater frequency than can be explained by the effects of errors associated with the ends of reads and surrounding sequence motifs that influence error frequencies. For example, errors are more likely at a position preceded by GG or following a number of GGC motifs, and errors are more likely towards the end of reads. For any given sequencing platform, systematic errors are individual base-call errors that non-randomly and disproportionately occur at specific genomic positions in assays on that sequencing platform but not other sequencing platforms.

As used herein, the term “empirical systematic error” refers to an error of FP at a genomic position that occurs at a frequency greater than 10%, 15%, 20% or 25%.

As used herein, a “platform-sample-target-dependent” filter is a filter which has different values for different sequencing platforms (e.g., a particular pyrosequencing platform vs. a particular sequencing-by-synthesis platform), different sample types (e.g., hepatoma vs. sarcoma biopsy; FF sample vs. FFPE sample), and/or a different sequencing target (e.g., a particular genetic locus or collection of loci). The term “platform-sample-target-dependent” filter includes “platform-dependent” filters, “sample-dependent” filters, “target-dependent” filters, “platform-sample-dependent” filters, “platform-target-dependent” filters, and “sample-target-dependent” filters. Each of these types of platform-sample-target-dependent filters can refer to the minimum allelic variant frequency (MVAF), minimum variant read threshold (MVRT), quality (QUAL) and systematic error (SE) filters.

As used herein, the term “actionable variant” means a variant that informs a particular course of diagnosis, prognosis, or treatment. The term “therapeutically actionable variant” means a variant that informs a particular course of treatment or therapy.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease, e.g., there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical arts for a particular disease or disorder, e.g., cancer.

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

As used herein, the terms “single nucleotide variant”, “SNV”, “single nucleotide polymorphism”, or “SNP” refer to a variation in a nucleotide sequence that occurs when a single nucleotide, e.g., A, T, C, or G, in a genome or other sequence differs between members of a particular species, or when a single nucleotide differs between paired chromosomes within an individual subject or patient. For example, two DNA oligonucleotide fragments from different subjects may contain a difference in a single nucleotide, such as the sequence TTCCT and TTCCG. In such an instance, there are two differing alleles, i.e., the “T allele” and the “G allele.” Typically, SNPs have only two alleles. Moreover, a subject may also be heterozygous or homozygous for a particular SNP. In this case, if the wild-type or naturally occurring allele is “TTC” at an “A-locus” and the subject has a sequence of “TTC” on one chromosome at the A-locus, and TTG on the other paired chromosome at the A-locus, then the subject is said to be heterozygous (C/G) at that locus. However, if the subject has a sequence of “TTG” on one chromosome at the A-locus, and TTG on the other paired chromosome at the A-locus, then the subject is said to be homozygous (G/G) at the A-locus.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder if, after receiving a therapeutic agent according to the methods of the present disclosure, the subject shows observable and/or measurable reduction in or absence of one or more signs and symptoms of a particular disease or condition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Overview of the Invention

Clinically, the goal of precision medicine is to improve treatment response and patient outcomes and to avoid unnecessary treatment when it is unlikely to be effective for a particular patient based on that patient's molecular profile. For example, cancer patients who are eligible for targeted therapies because therapeutically actionable variants are detected in their tumors have a 50%-70% response rate compared to patients who undergo “standard of care” chemotherapy regimens, where response rates are about 10%-20%. This substantial difference in treatment response underscores the need to increase the likelihood of accurately identifying actionable variants over variants of unknown clinical significance. In addition, it is important to confirm the absence of an allelic variant to reduce necessary treatment and ensure proper diagnosis and prognosis of patients. These concerns are not adequately addressed in the current art of laboratory-developed tests using NGS, where all detected variants are typically evaluated in the last step of the process to determine actionability.

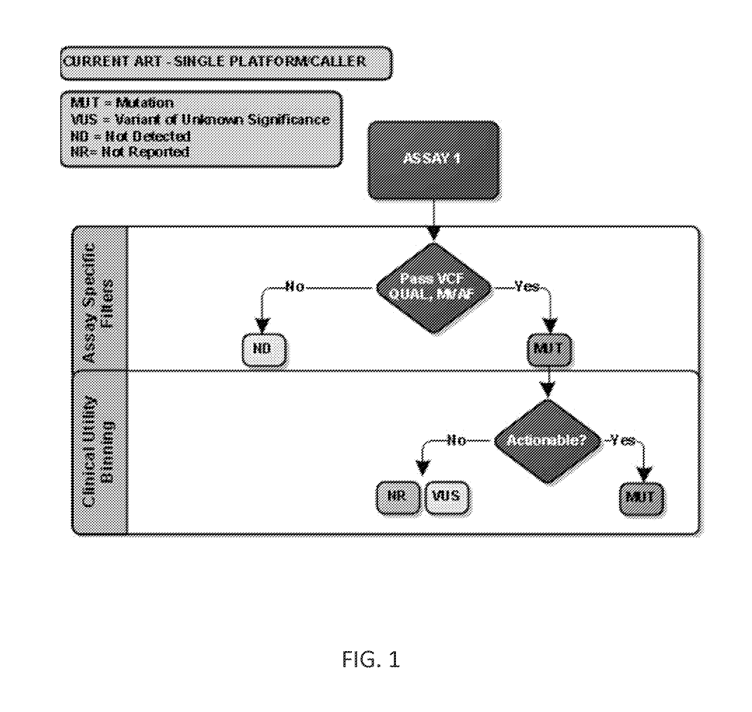

Unfortunately, NGS platforms produce false positive and false negative variant calls, limiting their usefulness for clinical testing. As shown in FIG. 1, the current state of the art is to employ a single sequencing technology and associated variant detector software that analyzes sequencing data and generates a variant call format (VCF) file. Assay-specific filters are used to filter out variants that fall below a (non-platform-sample-target-dependent) minimum variant allelic frequency (MVAF) and/or that have low quality scores (QUAL) as “not detected” (ND) (FIG. 1). While this approach may reduce the number of false positive calls, it also eliminates true positives that are clinically significant because they fall below the prescribed thresholds. Thus, it sacrifices sensitivity for PPV.

The present disclosure relates to methods and systems for analyzing genomic sequencing data to accurately detect the presence of genomic variants. Accurate variant detection helps identify the proper diagnosis, prognosis, and/or treatment for patients with particular conditions and disorders, such as cancer. The disclosed multi-platform variant detection methods and systems identify false negative calls and/or false positive calls made by state of the art NGS platforms by analyzing sequencing data produced by two different sequencing platforms, and applying to each set of sequencing data at least one filter, such as absence of a platform-dependent systematic error, a first platform-sample-target-dependent minimum variant read threshold, or a first platform-sample-target-dependent minimum variant allelic frequency. By using at least two sequencing platforms, and analyzing the sequencing data using the filters described herein, the present disclosure increases the accuracy of diagnosis, prognosis, and therapeutic regimes for various conditions, such as cancer, as compared to using current sequencing platforms and methods. As compared to current sequencing platforms, the present disclosure also allows for more accurate identification of therapeutic regimes or improved prognostics.

The methods described herein are designed to detect substitutions, duplications, insertions, deletions, indels, exon and gene copy number changes, select translocations, structural variants, SNVs and chromosome inversions and translocations, if present, in a biological sample from a subject. The samples include, but are not limited to, sputum, blood (or a fraction of blood such as plasma, serum, or particular cell fractions), lymph, mucus, tears, saliva, urine, semen, ascites fluid, whole blood, and biopsy samples of body tissue, as further discussed herein. In some embodiments, the sample is surgically resected cancerous tissue from a patient.

### Genomic Variant Detection Methods and Systems

The disclosed methods and systems allow analysis of sequencing data and genomic variant detection using more than one independent sequencing platform, including but not limited to NGS methodologies. In some embodiments, two different independent NGS methodologies are used; in other embodiments, three or more sequencing platforms are used.

In the disclosed methods and systems, sample, target and assay-specific post-analytic filters are applied to machine-readable output information (e.g., a SAM, BAM or VCF file) from at least two sequencing platform assays to increase PPV by reducing the number of false positive calls, and determining failed testing (i.e., QUAL and/or MVRT values below a certain value) at the variant level. Specifically, in certain implementations, VFC files and BAM files contain the machine-readable input information. A VCF file contains variant call information, while a BAM file contains sequence alignment data in a binary format. The detected variants are optionally categorized as actionable variants or non-actionable variants.

The invention is based, in part, on the selection and determination of values or thresholds for platform, sample and target-dependent filters, which include quality threshold (QUALT), minimum variant read threshold (MVRT), minimum variant allelic frequency (MVAF), and absence or reduction of systematic errors (SE). These filters are selected based on repeated empirical testing of specific nucleic acid targets using designated platforms and, optionally, specified tissue and fixation types.

The disclosed filters are compatible with any VCF, including those generated by vendor-supplied variant calling software. Alternatively, the methods can be practiced using SAM, BAM or equivalent files as the source of sequencing information.

In some embodiments, the disclosed methods and systems lower MVAF thresholds and increase PPV by including variants with very low VAFs (e.g., <2%) that would not normally be found in prior art VCFs with high MVAF thresholds (e.g., 5% or greater). Thus, in some embodiments, the methods of the invention permit genomic variant detection based on MVAFs less than 5.0%, 4.5%, 4.0%, 3.5%, 3.0%, 2.5%, 2.0%, 1.9%, 1.8%, 1.7%, 1.6% or 1.5%. In the prior art, increasing sensitivity in this manner comes at a significant cost to PPV because increasing the calls of low frequency true positive variants typically entails increasing the calls of low frequency false positive variants. For this reason, current single platform assays typically use more stringent filters to reduce false positives while understanding that true positives with relatively low VAFs (e.g., <5%) may also be incorrectly missed (i.e., false negatives).

In some aspects, in addition to the MVAF and QUAL filters, the development of the disclosed methods and systems led to the application of two additional filters to increase PPV or sensitivity: filtering out of systemic errors (SEs) and application of a minimum variant reads threshold (MVRT). SEs are recurrent false positive calls (e.g., >25%) that are repeatedly detected by one platform or the other. Without the use of the disclosed method and systems using at least two sequencing platforms, the SEs in the prior art methods lead to calling recurring variants that are, in fact, false positives. Similarly, the MVRT filter was established by examining the minimum number of variant reads required for 95% sensitivity at a specified level of coverage, where a majority of variants were at or near the threshold of detection (e.g., 1-5% VAF). Without the use of the disclosed MVRT filter, the prior art methods would result in the inclusion of more false positives and therefore lower PPV, unless a higher MVAF were employed, which would result in more false negatives and therefore lower sensitivity.

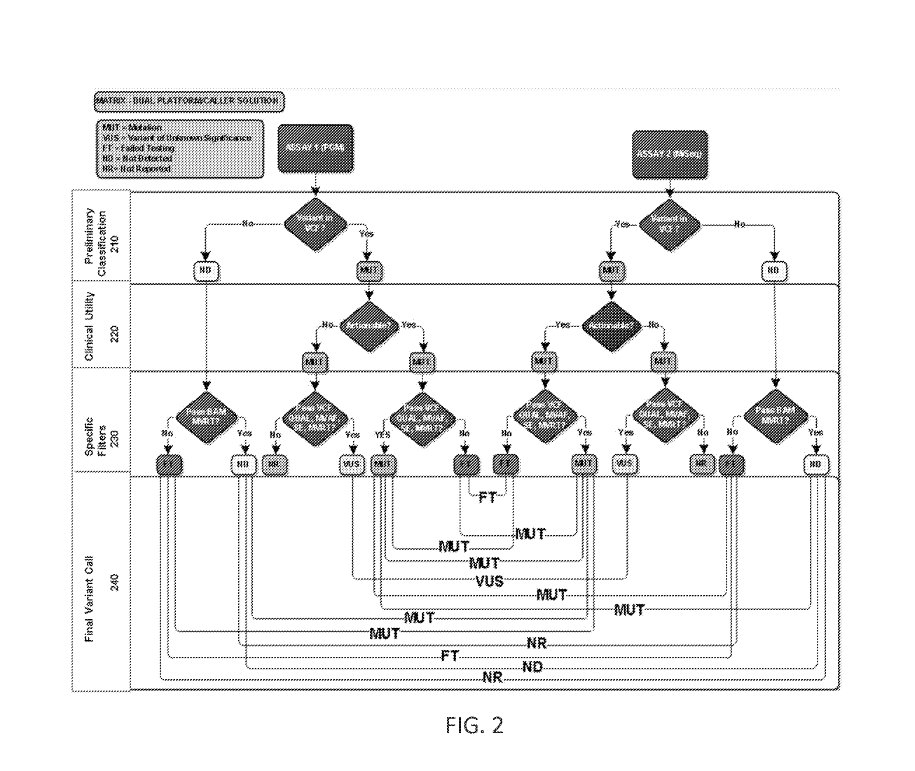

FIG. 2 is a schematic diagram showing embodiments of the disclosed methods. In some embodiments, during the step 210 of preliminary classification, the particular sequencing platform makes a preliminary classification based on VCF data across both platforms, whether a nucleic acid sequence is or is not a variant in a sample. A preliminary classification of variants as either a mutation (MUT) or not detected (ND) is made for each single platform. In certain implementations, the platform-specific application of an MVAF quality control is disabled at the platform level, prior to creation of the VCF. As described in more detail above, doing so increases the sensitivity of the platform but can result in false positive variant calls. In some embodiments, during the step 220 of considering clinical utility, variants are then classified as actionable or not actionable by referring to a predetermined knowledge base of therapeutic, prognostic, or diagnostic associations, e.g., by comparing the variants against a predefined list of variants. In some embodiments, considering clinical utility during this step significantly reduces the run time of variant calling by reducing the number of actionable variants that require further analysis. In step 230, specific filters are applied to increase the accuracy of the final variant call. Concordant non-actionable calls, which are non-actionable calls that are detected by both sequencing platforms, are considered variants of unknown significance (VUS). These detected variants can become actionable if future therapeutic, prognostic, or diagnostic associates are determined for that variant.

As shown in FIG. 2, if both platforms result in an actionable mutation, and both pass the designated number of MVRT, QUALT, SE, and MVAF filters, then the final variant call is a mutation (MUT). That both platforms confirmed the presence of a variant increases the confidence that the detected mutation is a true positive. For concordant non-actionable variant calls, if one call subsequently fails testing (FT) because it does not pass all of the filters, and the other call passes all of the filters, then these cases results in a status of not detected (NR). This reduces the number of false positives. In some embodiments, the disclosed methods and systems reduce the number of false negatives. For example, Assay 1 makes a preliminary classification of ND, but this passes the BAM and MVRT filters; Assay 2 then makes a preliminary classification of MUT, which is actionable, and this passes the VCF, QUAL, MVAF, SE, and MVRT filters. In this case, even though using Assay 1 would have resulted in a result of ND, analysis using Assay 2 results in a final variant call of MUT.

In the step 240 of the final variant call, the results can be divided into concordant MUT, concordant FT, or discordant variants, which include MUT/ND, ND/MUT, MUT/FT, or FT/MUT. For concordant actionable mutations that were identified as a MUT during preliminary classification 210, if any one of the QUAL, MVAF, SE, or MVRT filters is not passed for both platforms, then the result is FT. That both platforms failed the specific filters increases the confidence that the detected mutation was a false positive.

After the final variant call 240 using the disclosed methods and systems, actionable variants can optionally be manually reviewed by a qualified genome analyst, for example, a laboratory technician and pathologist. For concordant MUT calls, identification of the variant in two sequencing platforms, for example, both PGM and MiSeq platforms, is sufficient for confirmation. For discordant MUT calls, a third sequencing technology confirmation, for example, by either Sanger sequencing or pyrosequencing, can be performed. As shown in FIG. 2, for actionable variants, if Assay 1 passes the four filters, but Assay 2 does not pass the four filters, this can still result in a final variant call of a MUT. Thus, this exemplifies how the multi-platform variant detection methods and systems herein can resolve discordant calls made by different platforms, and accurately detect an allelic variant that is actionable. This final variant call can be confirmed through the use of a third sequencing platform or optionally through manual review.

To demonstrate the improvements to assay PPV and sensitivity made by the methods and systems discussed herein, a comparison was made between the results obtained with two prior art NGS systems, MiSeq and Ion PGM, and the current invention:

Methods.

As described in Examples 1 and 2 below, a Pooled Sample representative of variants at 20 cancer genes was used to empirically determine values for MVAF and MVRT which provide 95% confidence and 95% sensitivity for each of the MiSeq and PGM NGS platforms, using each of fresh frozen (FF) and formalin fixed/paraffin embedded (FFPE) samples. Thus, although the recommended or default values for the MiSeq platform use MVAF=0.05 and QUAL=100 filtering, the empirical validation determined that MVAF=0.017, QUAL=100, MVRT=5 filtering should be used for FF samples and MVAF=0.036, QUAL=100, MVRT=10 filtering should be used for FFPE samples. Similarly, although the recommended or default values for the PGM platform use MVAF=0.05 and QUAL=100 filtering, the empirical validation determined that MVAF=0.029, QUAL=100, MVRT=20 filtering should be used for FF samples and MVAF=0.018, QUAL=100, MVRT=21 filtering should be used for FFPE samples. The results are shown in the table below:

TABLE 1
Platform-Sample-Target Dependent Filters

| Platform | Sample Type | MVAF | MVRT |
|---|---|---|---|
| MiSeq | FF | 0.017 | 5 |
| PGM | FF | 0.029 | 20 |
| MiSeq | FFPE | 0.036 | 10 |
| PGM | FFPE | 0.018 | 21 |

Results.

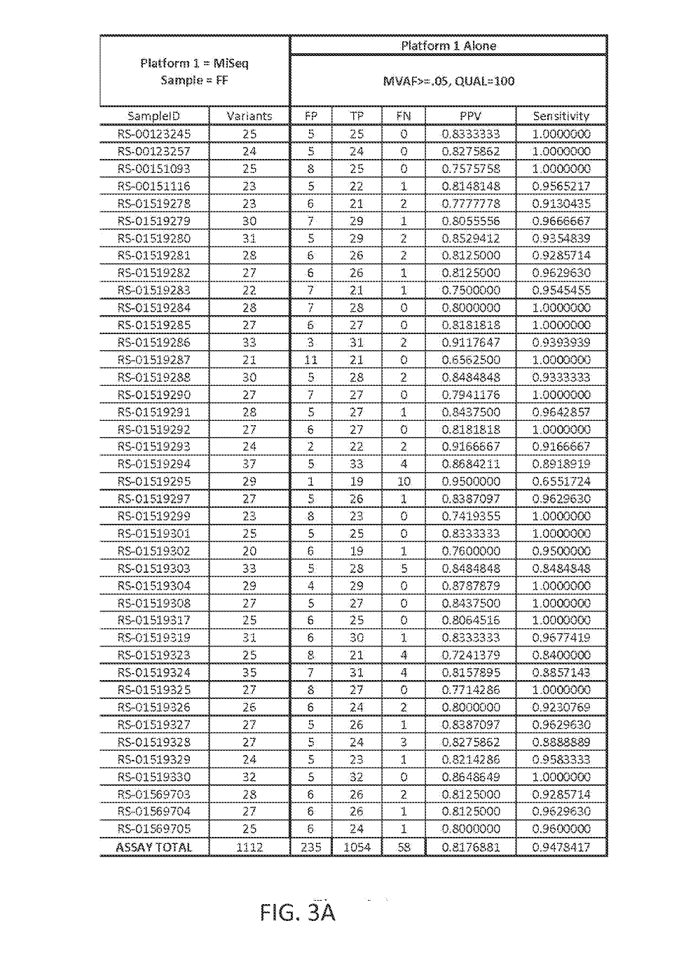

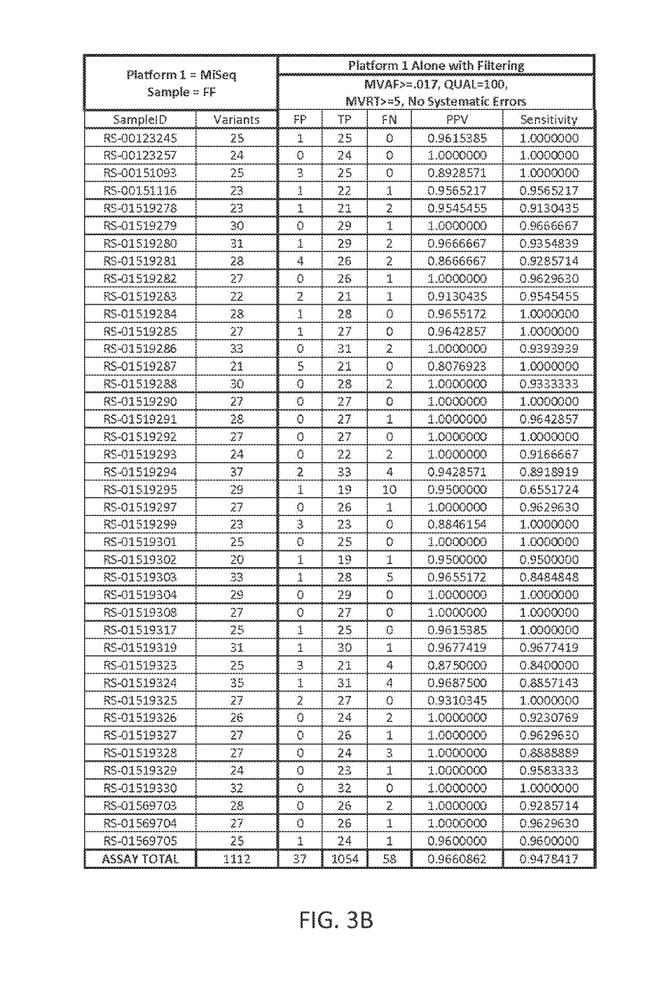

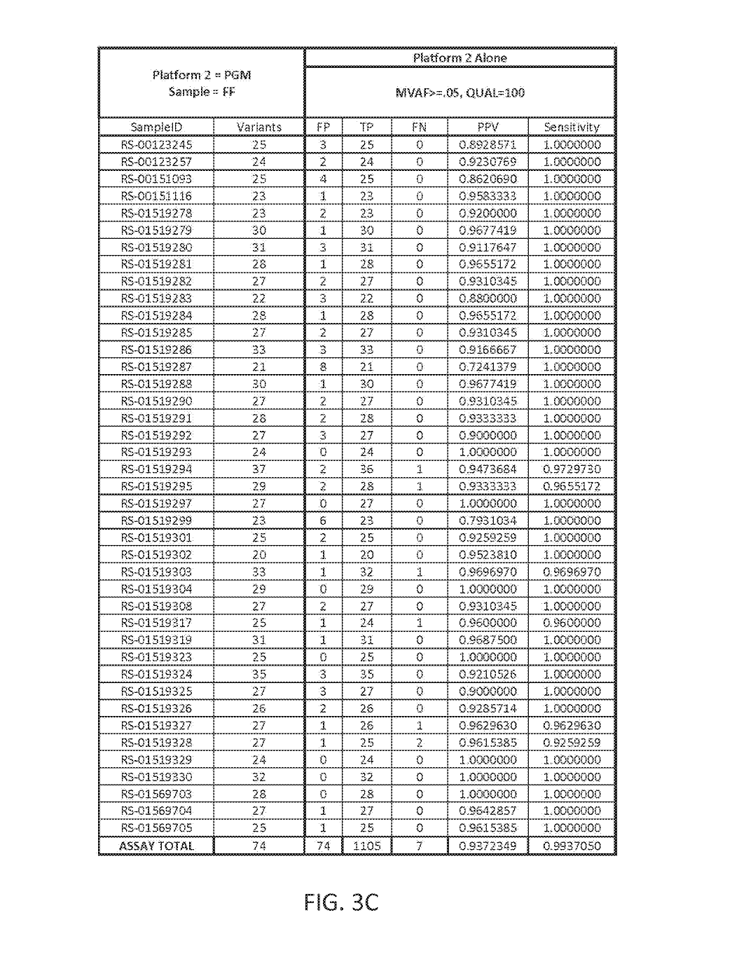

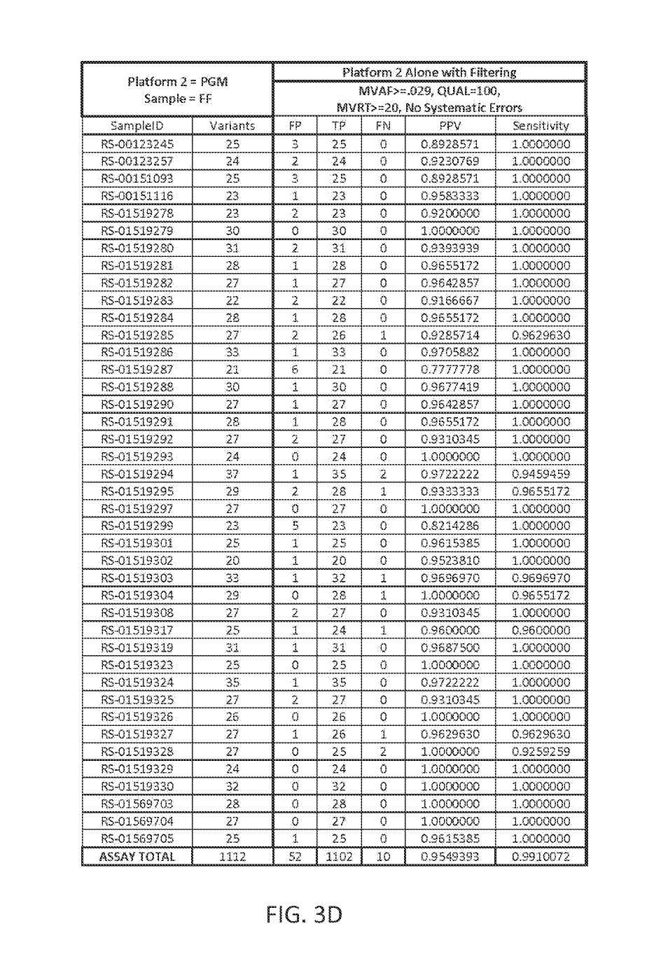

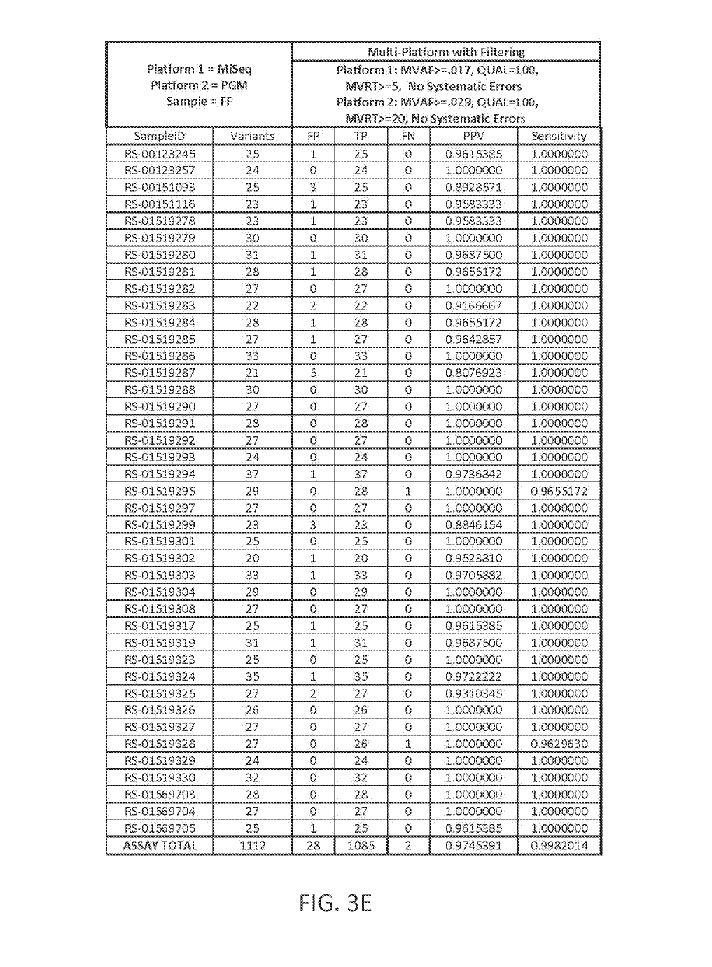

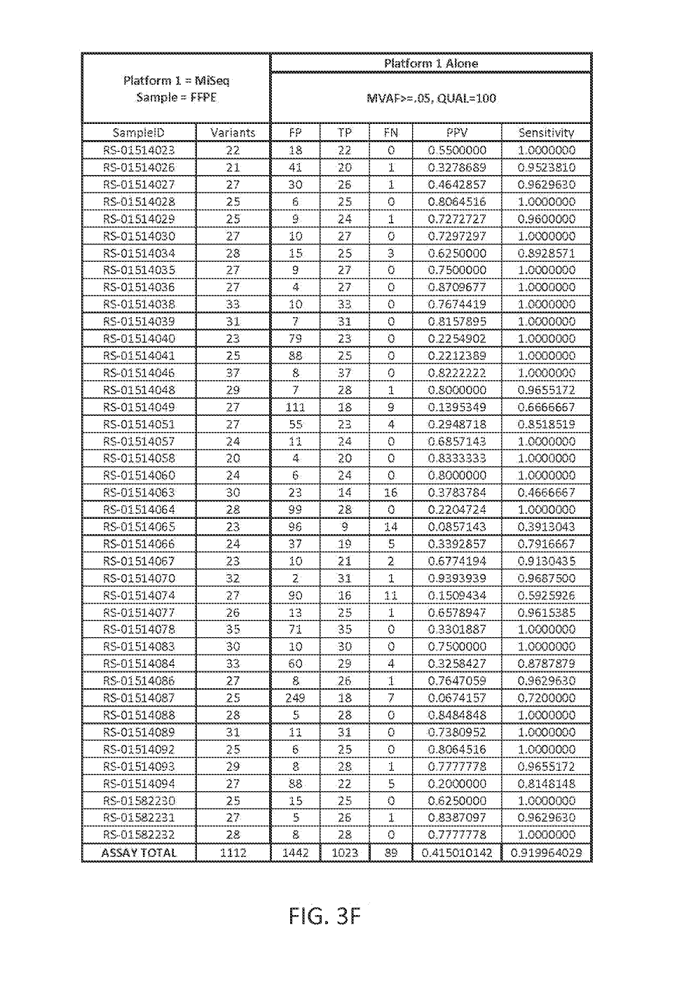

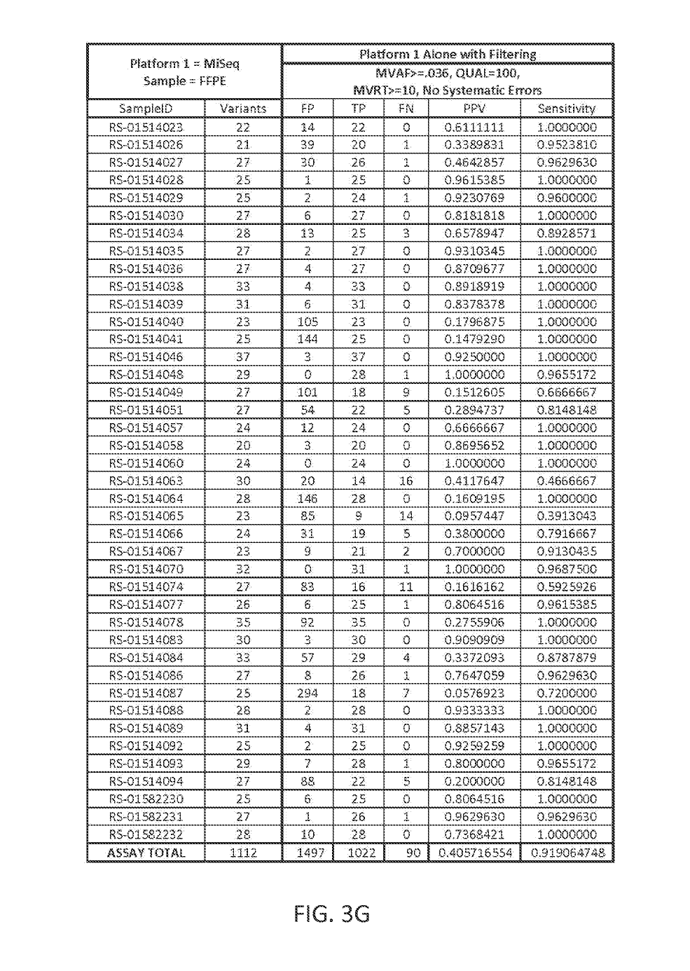

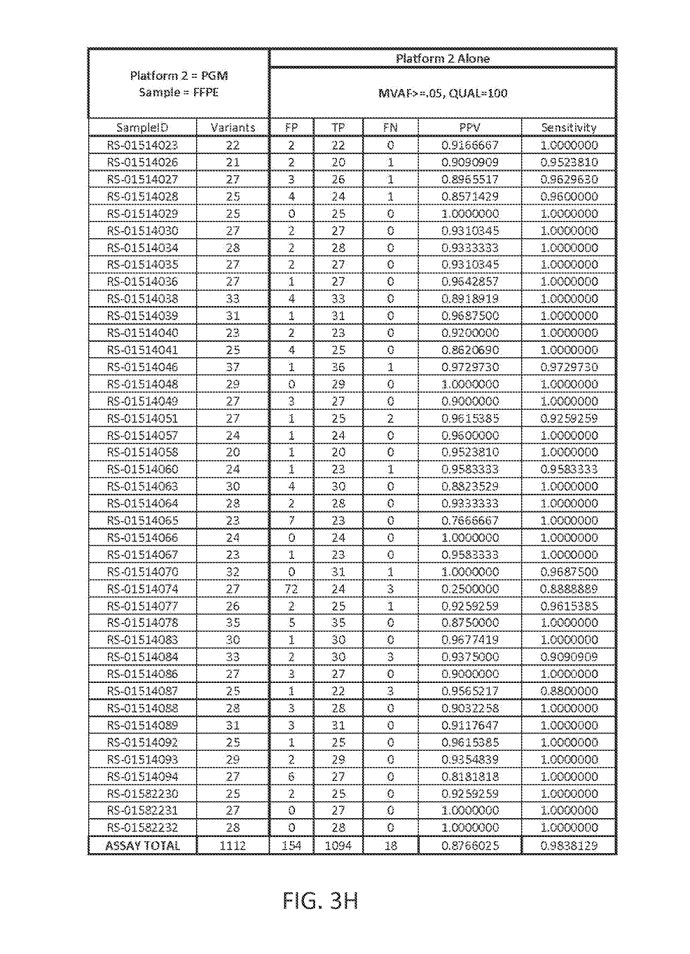

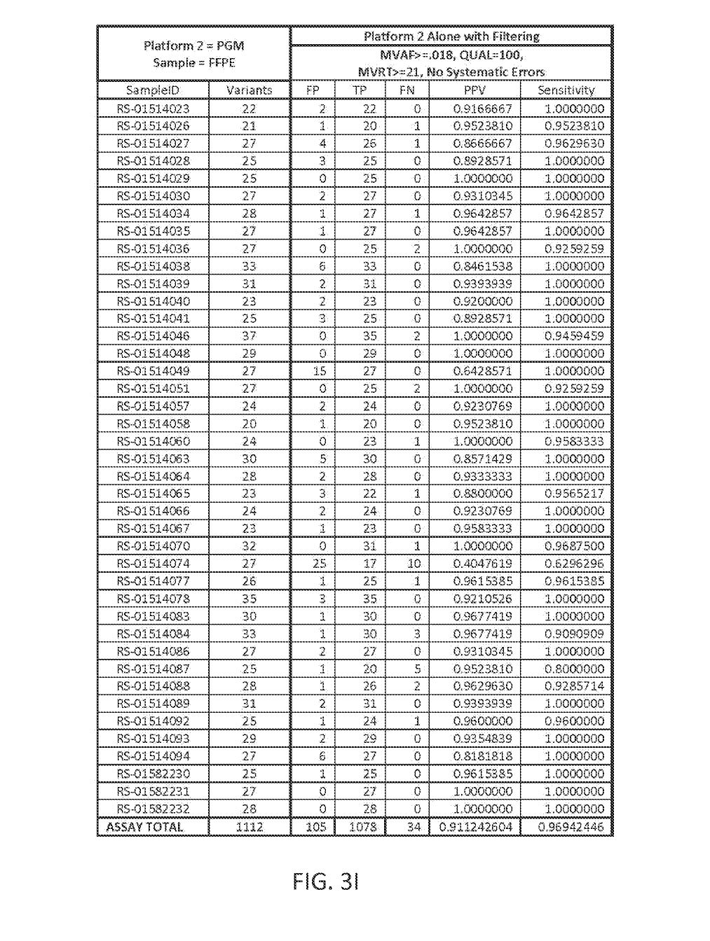

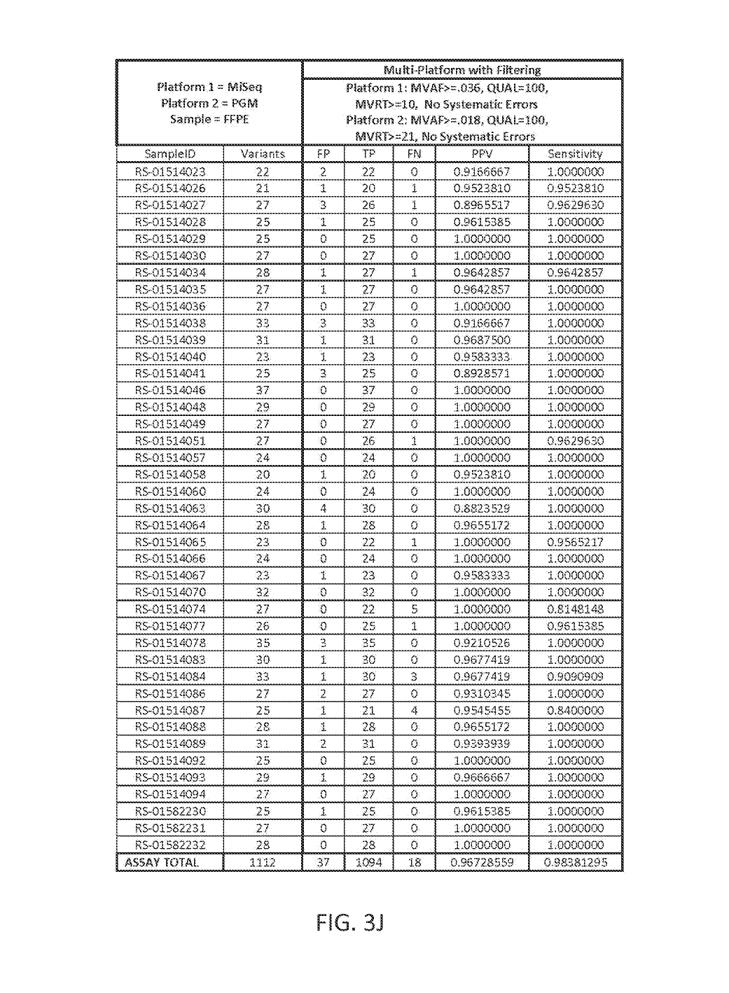

FIGS. 3A-J show the PPV and sensitivity for each of the 41 gold standard reference samples used in the SNV validation, as well as the PPV and sensitivity of each assay in its entirety, for both FF and FFPE tissue for each of these scenarios:

FIG. 3A: FF sample on MiSeq alone

FIG. 3B: FF sample on MiSeq with empirical MVAF, MVRT and QUAL filters

FIG. 3C: FF sample on PGM alone

FIG. 3D: FF sample on PGM with empirical MVAF, MVRT and QUAL filters

FIG. 3E: FF sample on dual MiSeq/PGM platforms with empirical MVAF, MVRT, QUAL and SE filters

FIG. 3F: FFPE sample on MiSeq alone

FIG. 3G: FFPE sample on MiSeq with empirical MVAF, MVRT and QUAL filters

FIG. 3H: FFPE sample on PGM alone

FIG. 3I: FFPE sample on PGM with empirical MVAF, MVRT and QUAL filters

FIG. 3J: FFPE sample on dual MiSeq/PGM platforms with empirical MVAF, MVRT, QUAL and SE filters

The empirical validation led to the determination that the MVAF could be lowered to improve PPV. However, applying the lower MVAF and MVRT filters without the QUAL and SE filters showed mixed results. For FF tissue, there were increases in PPV for both MiSeq and PGM, from 81.8% to 96.6% (FIGS. 3A and 3B) and 93.3% to 95.5% (FIGS. 3C and 3D), respectively. However, for FFPE, there was a decrease in PPV for MiSeq from 41.5% to 40.6% (FIGS. 3F and 3G) and an increase in PPV for PGM from 87.7% to 91.1% (FIGS. 3H and 3I). Changes in sensitivity were also mixed (see same Figures). However, application of dual-platform NGS platform-sample-target-dependent filters boosted both sensitivity and PPV (FIGS. 3E and 3J). Results were most dramatic for MiSeq FFPE, where PPV increased from 41.5% in the prior art (FIG. 3F) to 96.7% using the methods described herein (FIG. 3J). This 55.2% increase was an enormous improvement over the prior art, given that FFPE samples represent the overwhelming majority of samples submitted for NGS somatic variant detection.

TABLE 2
PPV and Sensitivity of NGS Assays: Current Art Compared to Multi-Platform
Variant Detection

| | | Prior Art | | Single Platform with | | Dual Platform with | |
|---|---|---|---|---|---|---|---|
| | | Single Platform with | | Empirical MVAF, | | Empirical MVAF, | |
| | | MVAF >= .05, | | MVRT, QUAL, and | | MVRT, QUAL, and | |
| Assay | | QUAL = 100 | | SE Filters | | SE Filters | |
| | Sample | Assay | Assay | Assay | Assay | Assay | Assay |
| Platform | Type | PPV | Sensitivity | PPV | Sensitivity | PPV | Sensitivity |
| MiSeq | FF | 0.818 | 0.948 | 0.966 | 0.948 | 0.975 | 0.998 |
| MiSeq | FFPE | 0.415 | 0.920 | 0.406 | 0.919 | 0.967 | 0.984 |
| PGM | FF | 0.937 | 0.994 | 0.955 | 0.991 | 0.975 | 0.998 |
| PGM | FFPE | 0.877 | 0.984 | 0.911 | 0.969 | 0.967 | 0.984 |

Differentiating variants that are actionable from those that are not prior to application of assay-specific filters removes poor-quality non-actionable MUT calls from consideration and they are not clinically reported. Additionally, assigning clinical actionability at this step differentiates mutations that are not detected from those that failed testing. This distinction is made in the disclosed methods and systems by reviewing the MVRT of an actionable variant at the BAM level, after which additional confirmation testing can be performed if detected in one platform and not the other. Dual assay testing as described in the methods of the invention also improves the likelihood of correctly reporting actionable variants. This was illustrated by showing results for two (2) actionable variants from two (2) patient samples in the clinical laboratory test validation (Table 3) that would have led to either missed treatment or unnecessary treatment if testing had occurred using only the MiSeq assay under prior art filters.

TABLE 3
Clinical Scenarios: Prior Art Compared
to Current Multi-Platform Methods

| | MiSeq Alone | | Current Method |
|---|---|---|---|
| MiSeq Alone | Clinical | Current Method | Clinical |
| Result | Scenario | Result | Scenario |
| (False) Negative¹ | Missed | True Positive | Proper Treatment |
| | Treatment | | |
| (False) Positive² | Unnecessary | Not Detected | No Unnecessary |
| | Treatment | | Treatment |

¹BRAF gene, V600E amino acid substitution, chr7: 140453136: A: T nucleotide substitution.
²KRAS gene, G12V amino acid substitution, chr12: 25398284: C: A nucleotide substitution.

### CONCLUSION

Applying multi-platform assay-specific filters as well as the methods described herein to validation data set reduced false positive and false negative calls, and increased assay PPV and sensitivity to acceptable diagnostic testing levels. These methods produced a final report of true positive mutations. The methods of the invention combined each of the two platform's filtered variants into a single final variant call (Mutation—Actionable, Mutation—VUS, Not Detected, Failed Testing, Not Reported). The methods and systems substantially reduced false negative calls, increased sensitivity and PPV at low VAF, within a turnaround time (TAT) that meets clinical requirements and NYS CLEP guidelines. The methods and systems distinguish failed testing from variants that are not detected. The methods and systems improved the confidence with which clinical decision making can be made.

### Systems For Genomic Variant Detection

In the disclosed methods and systems, first sequencing data produced by sequencing a first amplified nucleic acid sample derived from the biological sample using first sequencing platform is received; and second sequencing data produced by sequencing a second amplified nucleic acid sample derived from the biological sample using a second sequencing platform is received. The first sequencing platform is different from the second sequencing platform. The sequencing data include nucleotide sequences of a multiplicity of allelic variants, and the methods and systems include selecting from the allelic variants in the data at least one allelic variant for analysis, and calling the presence of the allelic variant in the biological sample if either (i) a first analysis of the first sequencing data relating to the allelic variant passes at least one filter selected from the group consisting of absence of a first platform-dependent systematic error, a first platform-sample-target-dependent minimum variant read threshold and a platform-sample-target-dependent minimum variant allelic frequency, or (ii) a second analysis of the second sequencing data relating to the allelic variant passes at least one filter selected from the group consisting of absence of a second platform-dependent systematic error, a second platform-sample-target-dependent minimum variant read threshold and a second platform-sample-target-dependent minimum variant allelic frequency.

In some aspects, the disclosure includes systems including a first interface for receiving first sequencing data indicative of a presence or absence of an allelic variant in a biological sample based on results from a first sequencing process performed on a first sequencing platform; and a second interface for receiving second sequencing data indicative of a presence or absence of the allelic variant in the biological sample based on results from a second sequencing process performed on a second sequencing platform, the first sequencing platform differing from the second sequencing platform. The system includes a computer-readable memory comprising a first filter based on base-pair level characteristics of variants detected in a reference sample by the first sequencing platform, wherein the first filter is selected from the group consisting of: a first minimum variant reads threshold; a first minimum variant allelic frequency; and a first set of systematic errors. In the system, the computer-readable memory comprising a second filter based on base-pair level characteristics of variants detected in a reference sample by the second sequencing platform, wherein the second filter is selected from the group consisting of: a second minimum variant reads threshold; a second minimum variant allelic frequency; and a second set of systematic errors. The system includes a computational engine comprising at least one computer processor. The computer-readable memory in the system includes instructions that when executed cause the computational engine to: conduct a first comparison of the first filter to the first sequencing data to determine if the data indicative of the presence or absence of the allelic variant passes the first filter; conduct a second comparison of the second filter to the second sequencing data to determine if the data indicative of the presence or absence of the allelic variant passes the second filter; and call the presence or absence of the allelic variant in the biological sample based on the results of the first comparison and the second comparison.

In the foregoing description, certain steps or processes can be performed on particular servers, computer platforms, or as part of a particular computing engine. These descriptions are merely illustrative, as the specific steps can be performed on various hardware devices, including, but not limited to, server systems and/or stand-alone computing platforms. Similarly, the division of where the particular steps are performed can vary, it being understood that no division or a different division is within the scope of the disclosure. Moreover, the use of “analyzer”, “module”, “engine”, and/or other terms used to describe computer system processing is intended to be interchangeable and to represent logic or circuitry in which the functionality can be executed.

In addition, certain implementations of the invention include machine-based hardware data interfaces that enable information (such as sequencing data) to be passed from one machine element to another machine element. For example, a computer system that contains analytic modules for filtering data according to the filters described herein and modules for reconciling the filtered results from multiple sequencing platforms has one or more hardware input interfaces for receiving the raw data from the sequencing platforms. Similarly, such as computer system has one or more output interfaces for providing the final variant call information to another machine element of an overall system. Optionally, the computer system can include one or more output modules that provide the final call information and/or other related information to a human/machine interface.

In some embodiments of the invention, any one or more of the input or output interfaces that enable the transfer of information between machine elements accept or provide information in a binary format (i.e., a format that, even when visually presented, is not human-readable). In other embodiments, the input or output interfaces accept or provide information in a machine readable format that is also human-readable when presented visually. In such cases, the modules for transforming the raw data into final variant call information can convert the information into a binary format for application of the methods disclosed herein, or the information can remain in the format provided. In any of the implementations or embodiments set forth herein, the information can remain in a digital format throughout the processing steps.

Illustrative examples of interfaces include, but are not limited to, serial computer interfaces (e.g., RS-232), parallel computer interfaces (e.g., IEEE 1284), Small Computer System Interface (SCSI) implementations, Universal Serial Bus (USB) interfaces, Firewire (IEEE 1394) interfaces, specialized Personal Computer Memory Card Interface Association (PCMCIA) adapter interfaces, network interfaces (e.g., Ethernet, token ring, etc.), and proprietary system interfaces (e.g., Apple, Inc. Thunderbolt interface). In addition, any of the interfaces can include data connections and supporting computer modules to retrieve or deposit computer files stored in non-transient memory, computer file storage systems, and/or computer-readable database/catalog systems.

The techniques and systems disclosed herein may be implemented as a computer program product for use with a computer system or computerized electronic device. Such implementations may include a series of computer instructions, or logic, fixed either on a tangible medium, such as a computer readable medium (e.g., a diskette, CD-ROM, ROM, flash memory or other memory or fixed disk) or transmittable to a computer system or a device, via a modem or other interface device, such as a communications adapter connected to a network over a medium.

The medium may be either a tangible medium and/or non-transient (e.g., optical or analog communications lines) or a medium implemented with wireless techniques (e.g., Wi-Fi, cellular, microwave, infrared or other transmission techniques). The series of computer instructions embodies at least part of the functionality described herein with respect to the system. Those skilled in the art should appreciate that such computer instructions can be written in a number of programming languages for use with many computer architectures or operating systems.

Furthermore, such instructions may be stored in any tangible memory device, such as semiconductor, magnetic, optical or other memory devices, and may be transmitted using any communications technology, such as optical, infrared, microwave, or other transmission technologies.

It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

In the present invention, at least two methods of sequencing known in the art can be used. In some embodiments, one or both of these platforms are “next generation sequencing” (NGS) platforms. NGS platforms employ high-throughput sequencing technologies that determine nucleotide sequences in a highly parallel fashion (e.g., greater than 150 molecules are sequenced simultaneously). NGS methods are known in the art, and are described, e.g., in Metzker (2010), Nature Biotechnology Reviews 11:31-46. NGS methods include single-molecule real-time sequencing (SMRT™, Pacific Bio, Pacific Biosciences, Menlo Park, Calif.), ion semiconductor sequencing (Ion PGM™, and Ion Proton™, Life Technologies Corp., Logan, Utah), pyrosequencing (454 Life Sciences, Roche Diagnostics Corp., Basel, Switzerland), sequencing by synthesis (HiSeg™ and MiSeg™, Illumina, Inc., San Diego, Calif.), sequencing by ligation ((SOLiD™, Life Technologies Corp. Logan, Utah)), and chain termination (Sanger sequencing). See, e.g., Quail et al. (2012), “A tale of three next generation sequencing platforms: comparison of Ion Torrent, Pacific Biosciences and Illumina MiSeq sequencers,” BMC Genomics 13(1). Although NGS technology has enhanced the speed of data acquisition, it presents serious problems in accurately analyzing massive amounts of sequencing data. Not only is the sheer amount of the data problematic, but NGS data has also been shown to be more error prone than previous first-generation sequencing technologies. Various NGS platforms and variant callers have both position-specific (depending on the location in the read) and sequence-specific (depending on the sequence in the read) errors. Different NGS platforms and variant callers can also have systematic errors associated with them.

NGS technologies typically include multiple steps, e.g., template preparation, sequencing and imaging, and data analysis. Methods for template preparation, moreover, can include multiple steps, such as randomly breaking nucleic acids (e.g., genomic DNA or cDNA) into smaller sizes and generating sequencing templates (e.g., fragment templates or mate-pair templates). The spatially separated templates can be attached or immobilized to a solid surface or support, allowing massive numbers of sequencing reactions to be performed simultaneously. Types of templates that can be used for NGS reactions include, e.g., clonally amplified templates originating from single DNA molecules, and single DNA molecule templates.

Methods for preparing clonally amplified templates include, e.g., emulsion PCR (emPCR) and solid-phase amplification. In emPCR, a library of nucleic acid fragments is generated, and adaptors containing universal priming sites are typically ligated to the ends of the fragment. The fragments then may be denatured into single strands and captured by beads. Each bead captures a single nucleic acid molecule. After amplification and enrichment of emPCR beads, a large number of templates can be attached or immobilized in a polyacrylamide gel on a standard microscope slide, chemically crosslinked to an amino-coated glass surface, or deposited into individual PicoTiterPlate (PTP) wells, in which the NGS reaction can be performed.

Solid-phase amplification can also be used to produce templates for NGS. Typically, forward and reverse primers are covalently attached to a solid support. The surface density of the amplified fragments is defined by the ratio of the primers to the templates on the support. Solid-phase amplification can produce hundreds of millions of spatially separated template clusters). The ends of the template clusters can be hybridized to universal sequencing primers for NGS reactions. Other methods for preparing clonally amplified templates also include, e.g., Multiple Displacement Amplification (MDA) (Lasken (2007), Curr Opin Microbial. 10(5):510-6). MDA is a non-PCR based DNA amplification technique. The reaction involves annealing random hexamer primers to the template and DNA synthesis by high fidelity enzyme, typically phi29 at a constant temperature. MDA can generate larger sized products with lower error frequency.

Template amplification methods such as PCR can be coupled with NGS platforms to target or enrich specific regions of the genome (e.g., exons). Exemplary template enrichment methods include, e.g., microdroplet PCR technology (Tewhey et al. (2009), Nature Biotech. 27:1025-1031), custom-designed oligonucleotide microarrays, and solution-based hybridization methods (e.g., molecular inversion probes (MIPs) (Porreca et al. (2007), Nature Methods, 4:931-936; Krishnakumar et al. (2008), Proc. Natl. Acad. Sci. USA, 105:9296-9310; Turner et al. (2009), Nature Methods, 6:315-316), and biotinylated RNA capture sequences (Gnirke et al. (2009), Nat. Biotechnol. 27(2): 182-9)

Single-molecule templates are another type of template that can be used for NGS reaction. Spatially separated single molecule templates can be immobilized on solid supports by various methods. In one approach, individual primer molecules are covalently attached to the solid support. Adaptors are added to the templates, and the templates are then hybridized to the immobilized primers. In another approach, single molecule templates are covalently attached to the solid support by priming and extending single-stranded, single molecule templates from immobilized primers. Universal primers are then hybridized to the templates. In yet another approach, single polymerase molecules are attached to the solid support, to which primed templates are bound.

Exemplary sequencing and imaging methods for NGS include, but are not limited to, cyclic reversible termination (CR1), sequencing by ligation (SBL), single-molecule addition (pyrosequencing), and real-time sequencing. CRT uses reversible terminators in a cyclic method that minimally includes the steps of nucleotide incorporation, fluorescence imaging, and cleavage. Typically, a DNA polymerase incorporates a single fluorescently modified nucleotide corresponding to the complementary nucleotide of the template base to the primer. DNA synthesis is terminated after the addition of a single nucleotide and the unincorporated nucleotides are washed away. Imaging is performed to determine the identity of the incorporated labeled nucleotide. Then in the cleavage step, the terminating/inhibiting group and the fluorescent dye are removed.

SBL uses DNA ligase and either one-base-encoded probes or two-base-encoded probes for sequencing. Typically, a fluorescently labeled probe is hybridized to its complementary sequence adjacent to the primed template. DNA ligase is used to ligate the dye-labeled probe to the primer. Fluorescence imaging is performed to determine the identity of the ligated probe after non-ligated probes are washed away. The fluorescent dye can be removed by using cleavable probes to regenerate a 5′-PO₄ group for subsequent ligation cycles. Alternatively, a new primer can be hybridized to the template after the old primer is removed.

Pyrosequencing methods are based on detecting the activity of DNA polymerase with another chemiluminescent enzyme. Typically, the method allows sequencing of a single strand of DNA by synthesizing the complementary strand along it, one base pair at a time, and detecting which base was actually added at each step. The template DNA is immobile, and solutions of A, C, G, and T nucleotides are sequentially added and removed from the reaction. Light is produced only when the nucleotide solution complements the first unpaired base of the template. The sequence of solutions which produce chemiluminescent signals allows the determination of the sequence of the template.

Other sequencing methods for NGS include, but are not limited to, nanopore sequencing, sequencing by hybridization, nano-transistor array based sequencing, polony sequencing, scanning tunneling microscopy (STM) based sequencing, and nanowire-molecule sensor based sequencing.

Nanopore sequencing involves electrophoresis of nucleic acid molecules in solution through a nano-scale pore which provides a highly confined space within which single-nucleic acid polymers can be analyzed. Exemplary methods of nanopore sequencing are described, e.g., in Branton et al. (2008), Nat. Biotechnol. 26(10):1146-53. Sequencing by hybridization is a non-enzymatic method that uses a DNA microarray. Typically, a single pool of DNA is fluorescently labeled and hybridized to an array containing known sequences. Hybridization signals from a given spot on the array can identify the DNA sequence. The binding of one strand of DNA to its complementary strand in the DNA double-helix is sensitive to even single-base mismatches when the hybrid region is short or is specialized mismatch detection proteins are present. Exemplary methods of sequencing by hybridization are described, e.g., in Hanna et al. (2000), J. Clin. Microbiol. 38 (7): 2715-21.

Polony sequencing is based on polony amplification and sequencing-by-synthesis via multiple single-base-extensions (FISSEQ). Polony amplification is a method to amplify DNA in situ on a polyacrylamide film. Exemplary polony sequencing methods are described, e.g., in US Patent Publication No. US 2007/0087362. Nano-transistor array based devices, such as Carbon NanoTube Field Effect Transistor (CNTFET), can also be used for NGS. For example, DNA molecules are stretched and driven over nanotubes by micro-fabricated electrodes. DNA molecules sequentially come into contact with the carbon nanotube surface, and the difference in current flow from each base is produced due to charge transfer between the DNA molecule and the nanotubes. DNA is sequenced by recording these differences. Exemplary Nano-transistor array based sequencing methods are described, e.g., in U.S. Patent Publication No. US 2006/0246497.

Scanning tunneling microscopy (STM) can also be used for NGS. STM uses a piezo-electric-controlled probe that performs a raster scan of a specimen to form images of its surface. STM can be used to image the physical properties of single DNA molecules, e.g., generating coherent electron tunneling imaging and spectroscopy by integrating scanning tunneling microscope with an actuator-driven flexible gap. Exemplary sequencing methods using STM are described, e.g., in U.S. Patent Publication No. US 2007/0194225. A molecular-analysis device which is comprised of a nanowire-molecule sensor can also be used for NGS. Such devices can detect the interactions of the nitrogenous material disposed on the nanowires and nucleic acid molecules such as DNA. A molecule guide is configured for guiding a molecule near the molecule sensor, allowing an interaction and subsequent detection. Exemplary sequencing methods using nanowire-molecule sensor are described, e.g., in U.S. Patent Publication No. US 2006/0275779.

Double-ended sequencing methods can be used for NGS. Double ended sequencing uses blocked and unblocked primers to sequence both the sense and antisense strands of DNA. Typically, these methods include the steps of annealing an unblocked primer to a first strand of nucleic acid; annealing a second blocked primer to a second strand of nucleic acid; elongating the nucleic acid along the first strand with a polymerase; terminating the first sequencing primer; deblocking the second primer; and elongating the nucleic acid along the second strand. Exemplary double ended sequencing methods are described, e.g., in U.S. Pat. No. 7,244,567.

### Sequencing Software

DNA sequence analysis is performed, in some embodiments, using a combination of software resources available on the genomic sequencing and analysis website, software freely available on the web. In some embodiments, sequence analysis and identification utilized MacVector, Excel (Microsoft) and programs available on the Galaxy server. See http://galaxyproject.org. In some embodiments, cell/tumor sample purifications and gene expression profiling are employed. In some embodiments, the Affymetrix U133Plus2.0 microarray system is used, as previously described in Zhan, F., et al. Blood 108, 2020-2028 (2006) and Shaughnessy, J. D., Jr., et al. Blood 109, 2276-2284 (2007). Signal intensities are preprocessed and normalized by GCOS1.1 software (Affymetrix). Whole-genome amplification (WGA) genotyping data is performed as appropriate, as described in Heard-Costa et al., Computer-Readable Memory and Computational Engines.

### EXAMPLES

The present disclosure is further illustrated by the following examples, which should not be construed as limiting the foregoing disclosure in any way.

### Example 1

### 20 Gene NGS1: A Multi-Platform Variant Calling System for 20 Cancer Genes

The methods and systems disclosed herein were applied in “20 Gene NGS1”, an NGS assay with 205 actionable mutations for 20 genes including AKT1, ALK, BRAF, CTNNB1, DDR2, EGFR, GNA11, GNAQ, ERBB2/HER2, JAK2, KIT, KRAS, MAP2K/MEK1, NRAS, PDGFRA, PIK3CA, PTEN, RET, SMAD4, and SMO, using two different NGS methodologies. To more accurately call the variants, a series of filters was used to further classify preliminary classifications from at least two high throughput genomic technologies.

General:

A multi-analyte genomic test was developed to detect single nucleotide variants (SNV) in 20 genes relevant to cancer treatment decisions. Unlike other multi-analyte tests, the assay analyzes genes for which there are defined, actionable mutations in terms of therapeutic decision-making. All genes for mutation testing by next generation sequencing are from My Cancer Genome (MCG)(www.mycancergenome.org), an online personalized cancer medicine resource that provides information about gene mutations in specific cancers at a single nucleotide variant level, and the related therapeutic implications including available clinical trials, referred to as “actionable mutations.” At the time of test validation, 171 actionable single nucleotide variants were defined for the genes, in addition to actionable insertions and deletions in ALK (exons 23, 24 and 25), EGFR (exons 19 and 20), ERBB2 (exon 20), KIT (exons 8, 9, 11, 13, 14 and 17) and PDGFRA (exons 12, 14 and 18) (Table 4).

TABLE 4
List of genes and association with specific
cancers included in 20-gene assay

| GENE | CANCER TYPE(S) |
|---|---|
| AKT1 | Breast Cancer, Colorectal Cancer, Lung Cancer |
| ALK | Anaplastic Large Cell Lymphoma, Inflammatory |
| | Myofibroblastic Tumor, Lung Cancer, Neuroblastoma, |
| | Rhabdomyosarcoma |
| BRAF | Colorectal Cancer, GIST, Lung Cancer, Melanoma, Ovarian |
| | Cancer, Thyroid Cancer |
| CTNNB1 | Melanoma |
| DDR2 | Lung Cancer |
| EGFR | Lung Cancer |
| ERBB2 | Breast Cancer, Gastric Cancer, Lung Cancer |
| GNA11 | Melanoma |
| GNAQ | Melanoma |
| JAK2 | Acute Lymphoblastic Leukemia |
| KIT | Acute Myeloid Leukemia, GIST, Melanoma, Thymic |
| | Carcinoma, |
| KRAS | Colorectal Cancer, Lung Cancer, Ovarian Cancer |
| MAP2K1 | Lung Cancer, Melanoma |
| NRAS | Colorectal Cancer, Lung Cancer, Melanoma |
| PDGFRA | GIST |
| PIK3CA | Breast Cancer, Colorectal Cancer, Lung Cancer, Ovarian |
| | Cancer |
| PTEN | Breast Cancer, Colorectal Cancer, Lung Cancer, Ovarian |
| | Cancer |
| RET | Thyroid Cancer |
| SMAD4 | Colorectal Cancer |
| SMO | Basal Cell Carcinoma, Medulloblastoma |

The 20-gene assay test used two different NGS methodologies for cross-confirmation to increase turnaround time (TAT) and minimize the problem of false positive calls and optimize assay positive predictive value and assay sensitivity. This method allowed for calling at least one defined allelic variant as Failed Testing (FT), Not Detected (ND), or Mutated (MUT). This parallel system used the Illumina MiSeq and Ion Torrent PGM platforms to sequence all specimens and employed a clinical-grade laboratory information management system that manages the workflow process and associated information from obtaining a specimen to sequencing analysis. The management system managed all pre-analytical variables, sequencing procedures, and bioinformatics analysis including mapping, alignment and variant calls.

By requiring variant calls to have parallel confirmation across the two platforms, this helps exclude random false positive calls. The two amplicon-based assays targeted the same genes and were used to independently detect and then cross-confirm the reportable variants from each platform.

Classification of Results:

This multi-platform method provided comprehensive diagnosis-centric results based on the clinical indication for testing. In some embodiments, output from the methods of the invention takes into account the non-actionable and actionable status at a specific nucleotide positions, and all actionable mutations are reported in the context of the tumor type tested and other tumor types.

Classification of MUT Results:

All MUT(s), whether concordant or discordant, for both sequencing platforms enter into a “Classify Calls” worklist, where they were binned into actionable for the tumor type tested versus all others based upon the most recent information from My Cancer Genome. This resulted in two separate MUT worklists that shared a related but distinctly separate manual review workflow by a board-certified pathologist with the appropriate molecular pathology certification. Optionally, manual review included a two-step process of 1) visual review of the pileups using an appropriate genome viewer, and 2) review of tabulated data about the variant call that included variables about the MUT call and the second best call at that specific nucleotide location. These variables for both the MUT call and the second best call include the variant allelic frequency (VAF), number of variant reads, number of reference reads, read Q score, base Q score, strand bias, and nucleotides two base pairs 5′ and 3′ of the variant call.

ND and FT Results for Actionable MUTs:

The MVRT is applied to the BAM or equivalent file for any actionable variant position not reported in the VCF. Any actionable variant not detected that has total reads greater than the MVRT is classified as not detected (ND), while those positions that fail to meet the MVRT would be reclassified as failed testing (FT).

MUT Results for Tumor Type Tested:

The worklist for actionable MUT(s) for the tumor type tested included both concordant and discordant calls that require a decision of PASS, FAIL, or CONFIRM. All other MUT(s), including non-actionable and actionable for other tumor types, required a decision of PASS or FAIL. The primary distinction of these two worklists is actionable MUT(s) for the tumor type tested was confirmed by at least two independent technologies of either PGM, MiSeq, Sanger sequencing or pyrosequencing. Nonactionable MUT(s) or actionable MUT(s) for other tumor types may or may not be confirmed by two independent technologies and are not confirmed by Sanger sequencing or pyrosequencing.

Concordant actionable MUT(s) for the tumor type tested were PASS if both calls passed manual review and were reported as actionable mutations detected for tumor type tested; otherwise, they were categorized as CONFIRM or FAIL. Optionally, discordant actionable MUT(s) for the tumor type tested was confirmed by a third sequencing platform, including pyrosequencing (PyroMark), fragment analysis, or Sanger sequencing (ABI 3130x1), and were subsequently reported in the same fashion. MUT(s) for the tumor type tested that failed quality control review or confirmation were reported as actionable mutations not detected for tumor type tested. Optionally, discordant actionable MUT calls for the tumor type tested were failed upon manual review if the singular variant call displayed specific quality control characteristics, but were passed if they were confirmed with an additional sequencing platform.

MUT Results for Other Tumor Types:

Concordant non-actionable MUT(s), or actionable MUT(s) for other tumor types, were PASS if both calls met the same previously-defined filters and reported as non-actionable mutations detected or actionable mutations detected for other tumor types; otherwise, they were FAIL with no additional confirmation.

### Example 2

### Validation of the 20 Gene NGS1 System

In embodiments of the claimed methods and systems, the 20 Gene NGS1 assay was the first clinical next generation sequencing (NGS) assay developed. The assay was used to increase sensitivity and PPV using NGS platforms on the PGM (Ion Torrent) and MiSeq (Illumina) platforms for the panel of 20 genes (AKT1, ALK, BRAF, CTNNB1, DDR2, EGFR, ERBB2 (HER2), GNA11, GNAQ, JAK2, KIT, KRAS, MAP2K(MEK1), NRAS, PDGFRA, PIK3CA, PTEN, RET, SMAD4, and SMO), which included 362 exons, 46,701 bases, and 205 actionable mutations. The bait for targeted enrichment for the PGM included Ion Torrent AmpliSeq (ITAS) chemistry for targeted sequencing of 20 genes using 806 amplicons (73,037 total bases) in two multiplex reactions. The bait for targeted enrichment for the PGM included the Illumina TruSeq Custom Amplicon (TSCA) chemistry for targeted sequencing of 20 genes using 674 amplicons (118,782 total bases) in a single multiplex reaction. Match fresh frozen (FF) and formalin fixed paraffin embedded (FFPE) specimens were used to test sensitivity and PPV as a single platform result and then compared to a combined result.

In some embodiments, the results were reported as modular clinical reports. The reporting tool provided the results of a single completed test, or it integrated the results of two or more completed tests included in 20 Gene NGS1 in the same report, providing the infrastructure for including additional molecular tests in a single clinical report.

The 20 Gene NGS1 assay described in this Example detected SNVs and indels in tumor DNA using two parallel but distinctly different NGS methodologies, Ion Torrent Personal Genome Machine (PGM) and Illumina MiSeq, that increased sensitivity and reduced false positive calls. Each patient tested for 20 Gene NGS1 had parallel Illumina MiSeq and Ion Torrent PGM sequencing performed for the same DNA sample to reduce the problem of false positive calls while optimizing sensitivity.

Specimen Type(s), Including Minimum Volumes/Amounts to Perform the Assay. The 20 Gene NGS1 validation used fresh frozen (FF) or formalin fixed paraffin embedded (FFPE) resection or needle core biopsy specimens. The specimen represented tumor of at least 25% neoplastic tumor nuclei and not more than 40% necrosis. At 25% neoplastic tumor nuclei, a heterozygous mutation will still be present at a 12.5% variant allelic frequency (VAF). 20 Gene NGS1 had a validated 95% sensitivity at a 3.6% VAF for FFPE specimens and 2.9% VAF for FF specimens. By setting the minimum neoplastic tumor nuclei threshold at more than twice our validated variant allelic frequency threshold, this allowed for an additional 50% tumor heterogeneity in the mutational status of the neoplastic cells for any given specimen.

Specimen Type(s). The preferred specimen for 20 Gene NGS1 was specific to the sequencing platform and tissue type and included: (1) for the MiSeq sequencing platform: 250 ng FF DNA or 500 ng FFPE DNA; and (2) for the PGM sequencing platform: 20 ng FF DNA or 40 ng FFPE DNA. Nucleic acid specimens were at a minimum DNA concentration of 10 ng/ul, as determined by picogreen fluorescence assay.

### Validation Overview

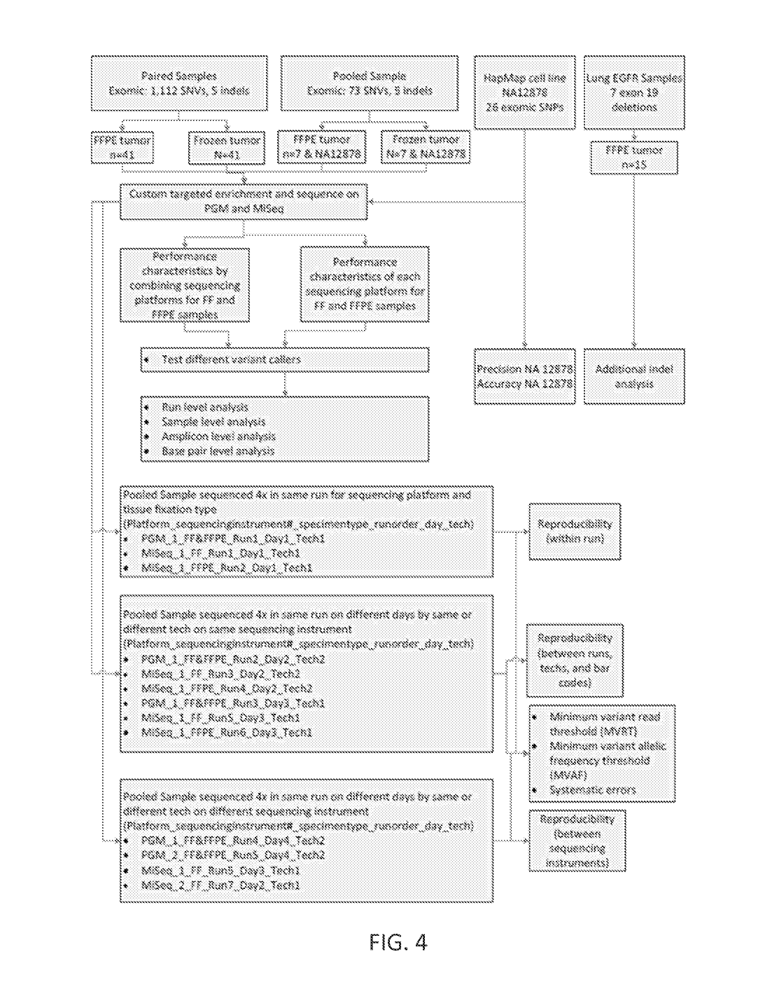

As shown in FIG. 4, which depicts the overall workflow for the 20 Gene NGS1 test validation, three gold standard sample sets (Paired Samples, Pooled Sample and NA12878) were used for defining the performance characteristics for the individual sequencing platforms. A fourth gold standard sample set, the EGFR Samples, was included to address limitations in analysis of indels in the other three sample sets, and specifically to address exon 19 EGFR microdeletions. Standard assay performance characteristics including assay and sample sensitivity and positive predictive value (PPV) for each platform (PGM and MiSeq) and tissue fixation type (FF and FFPE) were calculated from the Paired Samples test data, which contained gold standard variants most likely to represent the usual clinical scenario.

The assay performance characteristics of the Paired Samples were evaluated at the run, sample, amplicon, and base pair levels, with increasing granularity of the data at each successive evaluation. Run, sample, and amplicon validation parameters were platform and tissue fixation type-specific (PGM FF, PGM FFPE, MiSeq FF, MiSeq FFPE), while base pair level performance characteristics were calculated for individual sequencing platforms and across both platforms in the multi-platform variant detection methods and systems. For run and sample level performance, parameters were identified that are used to accept or reject an entire run, or an individual sample from further analysis. For amplicon performance evaluation, the results were not applicable to the daily sequencing controls, with the exception of failed amplicons.

Four base-pair level filters were developed to determine quality cutoffs for variant calls: (1) the minimum variant reads threshold (MVRT), (2) the minimum quality score threshold (QUALT), (3) the systematic error (SE) filter, and (4) the minimum variant allelic frequency threshold (MVAF). Due to the limited number of variants at or near the threshold of detection in the Paired Samples, the Pooled Sample was used to define performance MVRT and MVAF, both of which were designed to provide 95% confidence of variant calling at specified intervals of coverage as measures of analytical sensitivity and PPV. Compared to assay performance characteristics, which described the ability of an assay to detect variants across a wide spectrum of variant allelic frequencies, sensitivity and PPV as analytical evaluations were focused on the same or similar parameters when the majority of variants are at or near the threshold of detection. The MVRT defines a critical threshold for failed testing (FT) or not detected (ND) for variant calls with minimal variant reads at the lowest level of coverage. In a similar fashion, the MVAF defines a critical threshold for not detected for variant calls at higher levels of coverage. The Paired Samples data were used to define QUALT with the intent to maximize PPV while maintaining sensitivity. The Pooled Sample was also used to identify a subset of recurrent false positive variants that were platform-specific SEs, and that were subsequently used as a variant call quality filter.

In addition to analysis of individual sequencing platforms for the Paired Samples, performance across both platforms (PGM and MiSeq) was optimized using the methods and systems disclosed herein. The methods were developed to determine variant call quality by applying platform/specimen type quality control filters (i.e., MVRT, MVAF, QUALT and SE). The methods reclassified the single platform preliminary variant calls to conclude whether a preliminary variant classification was a true positive (TP), false negative (FN), or false positive (FP). In some embodiments, the results demonstrated that the disclosed multi-platform methods and systems cancelled out platform-specific false positives variant calls as random errors, while retaining the true positive variant calls, which were often concordant across the PGM and MiSeq platforms.

The Pooled Sample and NA12878 were used for multiple reproducibility evaluations. Precision, or the demonstration of consistent assay sensitivity across multiple runs, was assessed by including NA12878 in each sequencing run (PGM: 18 runs; MiSeq: 13 runs). As shown in FIG. 4, the Pooled Sample, which was sequenced four times in each run across multiple runs while changing one variable (day, instrument, technician, barcodes) each time, was important to defining various reproducibility endpoints. Reproducibility within one run or between multiple runs performed on different days by different technicians on different sequencing machines for each platform with different barcodes was evaluated using both the FF and FFPE Pooled Sample. For reproducibility within a run, the Pooled Sample was sequenced four times in the same run on the same day by the same technician. For the PGM runs, the FF and FFPE Pooled Sample are included in the same run on a single instrument. For the same specimens on the MiSeq the FF and FFPE Pooled Sample are in different runs on a single instrument due to assay interference of FF and FFPE specimens with the Illumina technology. For reproducibility between runs, the Pooled Sample was sequenced four times by the same technicians on the same instrument on a second day. In a parallel manner, the Pooled Sample was also sequenced four times on the same day and instrument, but by a different technician. For reproducibility between instruments, the Pooled Sample was sequenced four times in the same run on different days by the same or different technician and on different PGM or MiSeq instruments within our laboratory. For each run, the barcode indices were also rotated allowing for additional analysis for this variable.

All of the validation parameters were tested with multiple variant callers provided by Torrent Suite™ (Life Technologies Corp., Logan, Utah) and MiSeq Reporter™ (Illumina, Inc., San Diego, Calif.). By restricting our variant calling to these options, we used two well-described variant calling tools that are highly published in the public domain and are freely available; the Ion Torrent Variant Caller™ (ITVC) v3.6.63335 for Torrent Suite™ v3.6.2 and Somatic Variant Caller™ (SVC) v.2.1.12 for MiSeq Reporter™ v2.2.29. Sequencing was performed using the processes outlined in the user guides for these platforms.

In summary, the overall workflow for this validation utilized unique gold standard samples to optimize single sequencing platform sensitivity and PPV for a specific tissue fixation type (PGM FF, PGM FFPE, MiSeq FF, MiSeq FFPE) and then developed numerous run, sample, amplicon and base pair level thresholds and filters that were subsequently applied in the disclosed methods and systems to optimize dual-platform sensitivity and PPV. Evaluation of performance characteristics at the base pair level showed both the limitations of single platform sequencing, especially in terms of PPV, and the strength of the disclosed methods and systems to combine sequencing data from two sequencing platforms, and using the specific filters disclosed herein. While the pre-validation goal was to achieve 95% sensitivity and 95% PPV at a VAF of 5% or greater, the base pair performance analysis of the Pooled Sample more precisely defined these cut-offs for PGM FF, PGM FFPE, MiSeq FF and MiSeq FFPE, and demonstrated utility below this value with the proper filters.

### Base Pair Level Validation Parameters

All of the base pair level validation parameters were objective quantitative measurements, thresholds, or filters derived during the validation of 20 Gene NGS1 to define the performance characteristics of this test.

### Definition of Validation Parameters at the Base Pair Level

Assay and Sample Sensitivity:

Assay sensitivity is calculated for a given variant type (SNV or indel) on a specific sequencing platform (e.g., PGM or MiSeq) using a specified tissue fixation type (FF or FFPE) and diverse group of samples representative of the expected samples to be tested at a VAF in the test samples most likely to reflect the actual clinical scenario. Assay sensitivity outside of the methods described herein is defined as the ratio of total true positives to total true positives plus total false negatives for the Paired Samples resulting in unique values for each sequencing platform and tissue fixation type (FF vs. FFPE). Assay sensitivity uses the same values for calculations after variant calls from both sequencing platforms have been reduced to a single call as determined by the methods and systems described herein. Sample sensitivity was measured by calculating the ratio of true positives to true positive plus false negatives for an individual sample in the Paired Samples for each sequencing platform and tissue fixation type (FF vs. FFPE), both outside and within the multi-platform detection methods and systems. Mean, or average sample sensitivity outside the multi-platform detection system is the average of sample sensitivity for a given sequencing platform and specific tissue fixation type (PGM FF, PGM FFPE, MiSeq FF, MiSeq FFPE). Sample sensitivity is the average of sample sensitivity for a specific tissue fixation type (FF or FFPE).

Assay Specificity:

Assay specificity was not included in this validation as the number of true negatives in our targeted panel of 46,701 base pairs was so overwhelming in comparison to the number of potential false negatives that any reasonable test data would result in a value of >99% and be of extremely limited utility. We focused on assay positive predictive value and error rate as a more meaningful measure of NGS specificity.

Assay PPV:

Assay positive predictive value (PPV) was calculated for a given variant type (SNV or indel) on a specific sequencing platform (PGM or MiSeq) using either FF or FFPE specimens at a variant allelic frequency in the test samples most likely to reflect the actual clinical scenario. Assay PPV outside the multi-platform detection methods and systems is defined as the ratio of the sum of true positives to the sum of true positives plus the sum of false positives for the Paired Samples for each sequencing platform and tissue fixation type (PGM FF, PGM FFPE, MiSeq FF, MiSeq FFPE). Assay PPV within the multi-platform detection methods and systems utilizes the same values for calculations after variant calls from both sequencing platforms have been reduced to a single call following the application of the methods and systems disclosed herein. Sample PPV is defined as the ratio of true positives to true positive plus false positives for an individual sample in the Paired Samples for each sequencing platform and tissue fixation type (FF vs. FFPE) both outside and within the disclosed methods and systems. Mean sample PPV outside the disclosed methods and systems is the average of sample PPV for a given sequencing platform and specific tissue fixation type (PGM FF, PGM FFPE, MiSeq FF, MiSeq FFPE). Mean sample PPV within the disclosed methods and systems is the average of sample PPV for a specific tissue fixation type (FF or FFPE).

Quality Threshold (QUALT):

The quality threshold (QUALT) is defined as the minimum base pair quality score (QUAL) for which above or equal to that value variants in the VCF file will be accepted for final analysis, while below this value they are rejected. The intent of QUALT is to improve PPV at minimal to no impact on sensitivity. QUALT for SNV(s) or indels is measured by calculating the ratio of true positives to false positives for all SNV(s) or indels ranked by QUAL core using the Paired Samples. A ratio greater than one indicates false positives are excluded at the expense of true positives relative to the calculated value.

Minimum Percent Variant Reads (MPVR):

The minimum percent variant reads defines the variant allelic frequency cut-off for which there is 95% confidence that 95% of all SNV(s) are detected when half or more of the expected variants in the tested sample(s) are at or near the threshold of detection. The minimum percent variant read defines a threshold for which below that value there is less than 95% confidence that all variants in a given sample are detected. The MPVR is an equivalent measure of limits of detection for any given single variant. The MPVR is measured by first calculating percent variant reads required for 95% sensitivity for all variants within multiple specified intervals of reads using multiple replicates across multiple runs in the Pooled Sample. Measurement of the MPVR requires reduction in the dimensions of the data by first defining specified median read intervals of 1-50, 51-100, 101-150, 151-200, etc. Each variant is then ranked by descending predicted VAF. At each of these median total number of reads the VAF for which 95% sensitivity can be attained for all variants within this given region is then calculated. This percent variant reads required is then plotted against the specified median read intervals and the intersection of the two values represents the MPVR at that level of coverage.

Linear regression was then used to obtain the best fit for the line of observed plotted values to more accurately measure the MPVR at each specified interval of reads. The MPVR is independently calculated for each sequencing platform and tissue fixation type (PGM FF, PGM FFPE, MiSeq FF, MiSeq FFPE). MPVR could not be calculated for indels due to the limited number of these variants in the Pooled Sample.

Minimum Variant Reads (MVR):

The MVR defines the minimum number of variant reads at any given number of total reads for which there is 95% confidence that 95% of all SNV(s) are detected when half or more of the expected variants in the tested sample(s) or at or near the threshold of detection. The MVR is calculated in the same fashion as MPVR except that total variant reads is plotted against the specified median read intervals (FIG. 4). Linear regression is again used to more accurately measure the MVR at each specified interval of reads. Similar to MPVR, MVR could not be calculated for indels due to the limited number of these variants in the Pooled Sample.

Minimum Variant Read Threshold (MVRT):

The MVRT is defined as the greater of the minimum value of MPVR or MVR at 1-50 reads for each sequencing platform and tissue fixation type (PGM FF, PGM FFPE, MiSeq FF, MiSeq FFPE). In the validation setting, any true positive with fewer variant reads than the MVRT is reclassified as a false negative. A non-gold standard false positive that fails to meet the MVRT is reclassified as not reported (NR). In the validation setting, MVRT does not apply to false negatives as they are never reclassified to avoid spuriously inflating sensitivity. In the clinical setting MVRT is applied to any actionable or non-actionable MUT or actionable variant not detected (ND). Any MUT, actionable or non-actionable, or actionable variant not detected that fails to meet the MVRT would be reclassified as failed testing (FT) within the multi-platform detection methods and systems.

Minimum Variant Allelic Frequency (MVAF):

The MVAF precisely defines the minimum variant allelic frequency for which 95% sensitivity is obtained at any level of coverage. In contrast to MPVR, MVR, and MVRT which evaluate sensitivity as percent or total variant reads versus total reads integrating various levels of coverage, MVAF directly evaluates sensitivity versus VAF at any level of coverage using the Pooled Sample. The MVAF is an equivalent measure of analytical sensitivity for all variants in a given sample. The MVAF is measured by plotting cumulative sensitivity versus VAF for a descending ranking of VAF for all variants in the Pooled Sample using multiple replicates across multiple runs. That minimum VAF value for which 95% sensitivity can no longer be obtained represents the MVAF. The MVAF could be readily applied to SNV(s) due to their abundance, but due to the paucity of indels and their extremely low VAF in the Pooled Sample, this value could not be directly determined in the same fashion. MVAF for indels utilized the Paired Samples and was defined as that minimum VAF for which 95% sensitivity could still be attained. No MVAF for indels could be determined for PGM FF or PGM FFPE due to lack of sensitivity, as opposed to MiSeq or the multi-platform detection methods and systems.

Minimum Variant Reads Positive Predictive Value (MVR-PPV):

The MVR-PPV is the minimum number of variant reads, independent of total coverage that allows for 95% confidence that a call is a true positive and not a false positive. As opposed to MVAF which compares sensitivity to VAF, the MVR-PPV is focused on PPV instead of total variant reads. MVR-PPV is measured by calculating cumulative PPV of the Pooled Sample GS for a descending ranking of all variants by number of variant reads and then determining the number of variant reads where PPV is below 95%. In the validation results the values of MVR-PPV for PGM are very close to those for the MVRT, but much higher for MiSeq, limiting the usefulness of this parameter and underscoring the importance of the multi-platform detection methods and systems in managing false positive calls in next generation sequencing.

Systematic Errors:

Systematic errors (SE) are recurrent false positive (FP) variants, defined as being present in at least 25% of all replicates of the Pooled Sample for a specific sequencing platform (PGM or MiSeq) using either FF or FFPE specimens. All systematic errors in the clinical setting that correspond to an actionable variant are reported as failed testing.

### Base Pair Level Performance Characteristics

Performance characteristics at the base pair level can be divided into those that are used for thresholds or filters (MVRT, QUALT, SE, MVAF), reproducibility, and measurements of the output (sensitivity and PPV) of a single or combined sequencing platform.

### Minimum Variant Reads Threshold (MVRT)

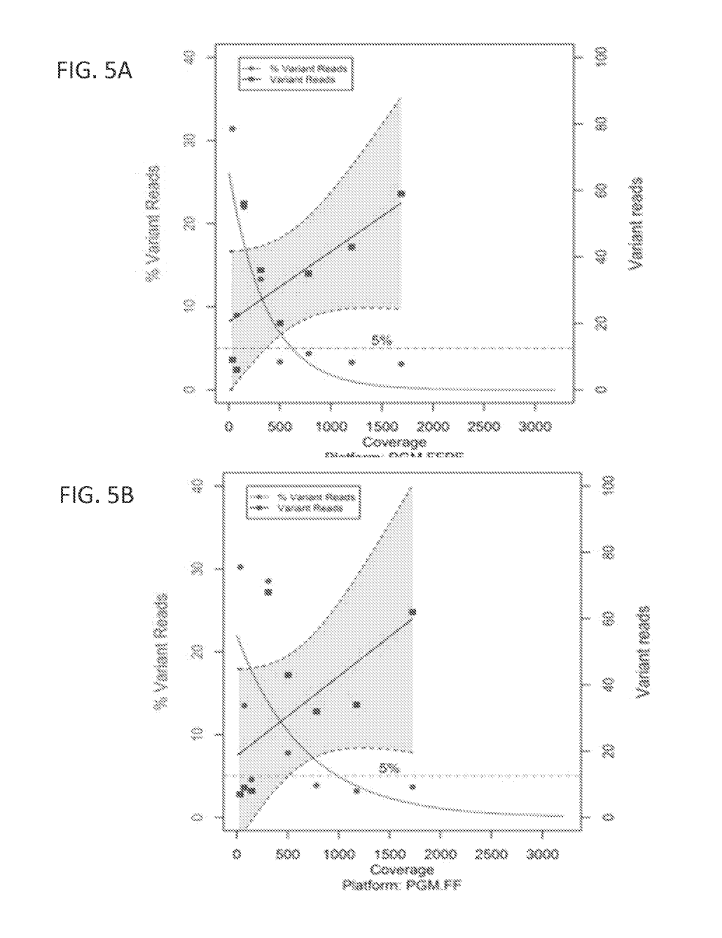

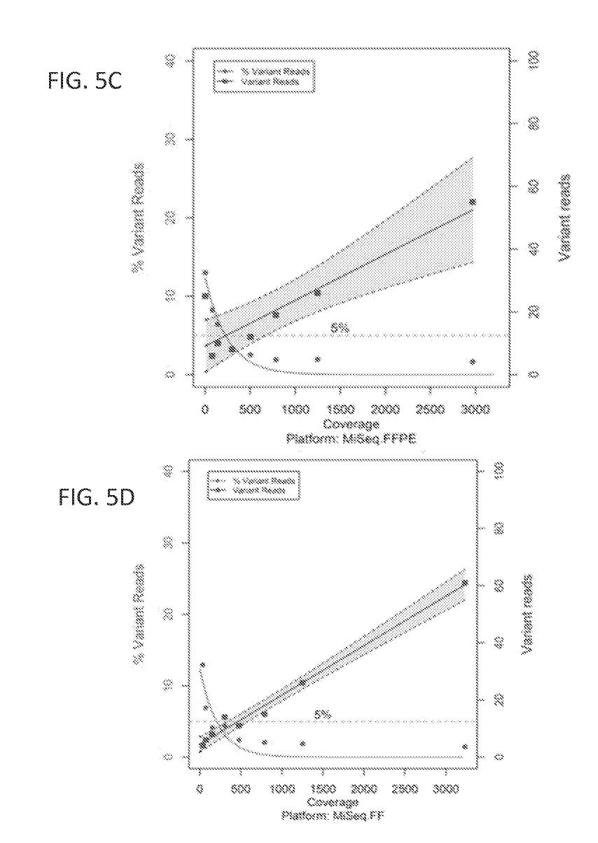

As a first requirement for calculating any of the performance characteristics of 20 Gene NGS1, a minimum variant reads threshold (MVRT) was established by evaluating the minimum number of variant reads (MVR) required for 95% sensitivity at a specified level of coverage. This analysis was performed using multiple replicates of the Pooled Sample where a majority of variants were at or near the threshold of detection and variant calling performed using SVC v.2.1.12 and ITVC v.3.6.63335. While four different variant callers (GATK (MiSeq), SVC v.2.1.12 (MiSeq), ITVC v. 3.4.51874 (PGM), and ITVC v. 3.6.63335 (PGM)) were tested, the two variant callers with the optimal sensitivity—SVC v.2.1.12 and ITVC v. 3.6.63335—were chosen for development of thresholds and filters at the base pair level and final use in 20 Gene NGS1. The minimum variant reads required for 95% sensitivity was evaluated at specified intervals of coverage for both percent variant reads required and total number of variant reads required. The specified intervals of level of coverage were defined using specified median read intervals of 1-50, 51-100, 101-200, 201-400, 401-600, 601-1,000, 1,001-1,500 and 1,500 to 5,000. At each of these median total number of reads, the VAF for which 95% sensitivity could be attained for all variants within this given region was predicted for both total and percent variant reads. Each of these values was then plotted against the specified median read intervals and loess regression used to plot the best-fitted line (FIGS. 5A-D).

As shown in FIGS. 5A-D and Table 5, the predicted percent variant reads (MPVR) required for 95% sensitivity for SNV(s) at the 1-50 median reads interval ranged from a high of 24% for PGM FFPE to a low of 11% for MiSeqFFPE and FF. The corresponding value for the predicted total variant reads (MVR) for SNV(s) ranged from a high of 20 of PGM FFPE to a low of 5 for MiSeq FF. For both PGM and MiSeq the values for MVR and MPVR at 1-50 reads were very similar for FF and FPPE. The higher of these two values for each platform and specific tissue fixation type (FF or FFPE) at 1-50 reads was chosen as the threshold for MVRT (Table 5) for SNV(s). This value ranged from a high of <21 for PGM FFPE to a low of <5 MiSeq FF and FFPE. In a corresponding fashion the MVR and MPVR were predicted for the remaining higher intervals of coverage. As the targeted range of coverage of 200 to 400× to much higher coverage of 1,000 to 1,500× increased the spread between the predicted number of reads for MPVR and MVR widened. This was particularly prominent with MiSeq FF and FFPE, where only one variant read based upon percent variant reads was required for the MPVR, while greater than 20 were required for both for MVR by total variant reads.

TABLE 5
Predicted percent and total variant reads required
for 95% sensitivity required to pass the MVRT.

| Interval of | | PGM | PGM | MiSeq | MiSeq |
|---|---|---|---|---|---|
| coverage | Analysis | FFPE | FF | FFPE | FF |
| 1-50 | Predicted % variant reads (MPVR) | 24% | 21% | 11% | 11% |
| reads | Calculated MVR for % variant reads | 6 | 5 | 3 | 3 |
| | Predicted minimum variant reads (MVR) | 20 | 19 | 9 | 4 |
| | Minimum variant read threshold (MVRT) | <21 | <20 | <10 | <5 |
| 200-400 | Predicted % variant reads (MVR) | 12% | 14% | 4% | 3% |
| reads | Calculated MVR for % variant reads | 35 | 42 | 11 | 10 |
| | Predicted minimum variant reads (MVR) | 27 | 26 | 13 | 10 |
| 1,000-1,500 | Predicted % variant reads (MVR) | 0.90% | 3.40% | 0.97% | 0.45% |
| reads | Calculated MVR for % variant reads | 11 | 42 | 1 | 1 |
| | Predicted minimum variant reads (MVR) | 47 | 49 | 27 | 26 |

While it would be possible to derive a MVRT for each specified interval of coverage a more practical and precisely defined threshold for this purpose for SNV(s) was the MVAF. An MVRT could not be specifically defined for indels due to the limited number of such variants that did not allow for grouping by specified intervals of reads. As a default, we accepted all values for MVRT for indels of those for SNV(s) for the corresponding sequencing platform and tissue fixation type. In support of this default MVRT for indels there were other base pair level thresholds for SNV(s) and indels that were very similar including the MVAF for indels and SNV(s) for MiSeq and QUALT for both PGM and MiSeq.

### Minimum QUAL Threshold (QUALT)

As a second requirement for calculating any of the performance characteristics of 20 Gene NGS1, a mean read quality threshold (QUALT) was established by calculating the ratio of true positives to false positives for all SNV(s) ranked by QUAL score for the Paired Samples using MiSeq SVC v.2.1.12 and ITVC v3.6.63335. A ratio greater than one indicates false positives are excluded at the expense of true positives relative to the calculated value. The QUAL threshold (QUALT) is the minimum base pair quality score used to exclude false positive variants with minimal impact to sensitivity. For MiSeq FFPE the ratio of true positives to false positives SNV(s) did not exceed 1.0 at any QUAL score. For PGM FFPE, PGM FF, and MiSeq FF this value was only exceeded at a QUAL score of 100. For simplification of the data, the true positive and false positive SNV(s) were grouped by intervals of QUAL scores and a ratio of true positives to false positives calculated for each interval (Table 6).

TABLE 6
Ratio of true positive (TP) to false positive
(FP) SNVs for QUAL score in the Paired Samples.

| | | | True | False | |
|---|---|---|---|---|---|
| Platform | Sample Type | Qual Group | Positive | Positive | Ratio TP/FP |
| MiSeq | FFPE | 20-29 | 2 | 6240 | 0.000 |
| MiSeq | FFPE | 30-39 | 4 | 3048 | 0.001 |
| MiSeq | FFPE | 40-49 | 1 | 1836 | 0.001 |
| MiSeq | FFPE | 50-59 | 3 | 1312 | 0.002 |
| MiSeq | FFPE | 60-69 | 2 | 850 | 0.002 |
| MiSeq | FFPE | 70-79 | 2 | 643 | 0.003 |
| MiSeq | FFPE | 80-89 | 4 | 531 | 0.008 |
| MiSeq | FFPE | 90-99 | 3 | 362 | 0.008 |
| MiSeq | FFPE | >=100 | 1023 | 1741 | 0.588 |
| MiSeq | FF | 20-29 | 4 | 662 | 0.006 |
| MiSeq | FF | 30-39 | 1 | 105 | 0.010 |
| MiSeq | FF | 40-49 | 1 | 34 | 0.029 |
| MiSeq | FF | 50-59 | 4 | 15 | 0.267 |
| MiSeq | FF | 60-69 | | 3 | 0.000 |
| MiSeq | FF | 70-79 | 2 | 1 | 2.000 |
| MiSeq | FF | 80-89 | 6 | | NA |
| MiSeq | FF | 90-99 | 1 | | NA |
| MiSeq | FF | >=100 | 1054 | 37 | 28.486 |
| PGM | FFPE | <10 | | 359 | 0.000 |
| PGM | FFPE | 10-19 | | 846 | 0.000 |
| PGM | FFPE | 20-29 | | 547 | 0.000 |
| PGM | FFPE | 30-39 | 1 | 393 | 0.003 |
| PGM | FFPE | 40-49 | | 242 | 0.000 |
| PGM | FFPE | 50-59 | 1 | 141 | 0.007 |
| PGM | FFPE | 60-69 | | 115 | 0.000 |
| PGM | FFPE | 70-79 | 1 | 76 | 0.013 |
| PGM | FFPE | 80-89 | 2 | 40 | 0.050 |
| PGM | FFPE | 90-99 | 1 | 31 | 0.032 |
| PGM | FFPE | >=100 | 1095 | 166 | 6.596 |
| PGM | FF | <10 | | 66 | 0.000 |
| PGM | FF | 10-19 | | 20 | 0.000 |
| PGM | FF | 20-29 | | 2 | 0.000 |
| PGM | FF | 30-39 | | 1 | 0.000 |
| PGM | FF | 50-59 | | 1 | 0.000 |
| PGM | FF | 60-69 | 1 | 3 | 0.333 |
| PGM | FF | 80-89 | 1 | 1 | 1.000 |
| PGM | FF | >=100 | 1105 | 52 | 21.250 |

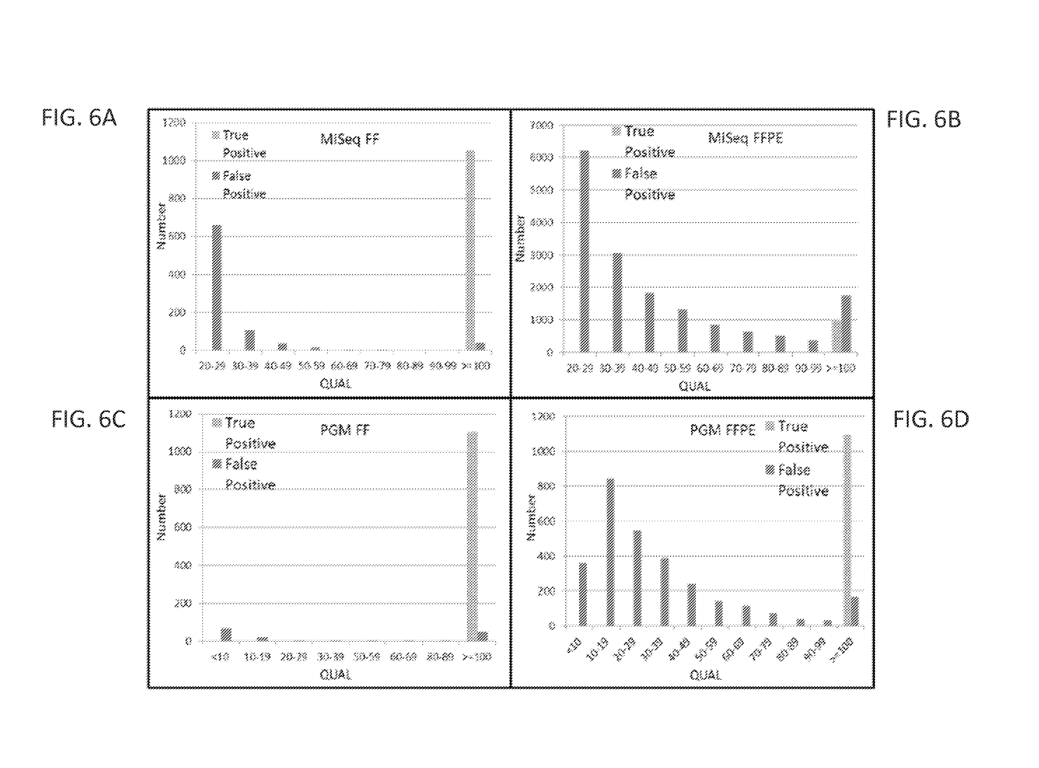

From these grouped intervals, it was apparent that using a QUALT of 100 for SNV(s) for PGM FF, PGM FFPE, MiSeq FF, and MiSeq FFPE resulted in a substantial decrease in false positives while minimally impacting true positives (FIGS. 6A-D). FIGS. 6A-D show that a quality (QUAL) score of 100 for single nucleotide variants (SNVs) for PGM FF, PGM FFPE, MiSeq FF, and MiSeq FFPE resulted in a substantial decrease in false positives while minimally impacting true positives.

For indels, the number of calls was much more limited than for SNV(s) allowing for a limited analysis of variant calls that have a QUAL=100. The analysis was also simplified as there were only five gold standard indels in the Paired Samples, of which all were detected for MiSeq FF and MiSeq FFPE, while three of the five were detected for both PGM FF and PGM FFPE. For PGM FF and PGM FFPE the ratio of true positives to false positives equaled 1.0 for QUAL equal to 100 (Table 7), while there were no true positive calls with a QUAL less than 100. In a similar fashion there were no true positive calls for MiSeq FF or MiSeq FFPE with a QUAL less than 100, although the ratio of true positives to false positives did not equal or exceed 1.0 for variant calls with a QUAL equal to 100.

TABLE 7
Ratio of true positive (TP) to false positive (FP)
indels for QUAL score in the Paired Samples.

| | | | True | False | |
|---|---|---|---|---|---|
| Platform | Type Sample | Qual Group | Positive | Positive | Ratio TP/FP |
| MiSeq | FFPE | <100 | 0 | 258 | 0.000 |
| MiSeq | FFPE | >=100 | 5 | 20 | 0.250 |
| MiSeq | FF | <100 | 0 | 134 | 0.000 |
| MiSeq | FF | >=100 | 5 | 12 | 0.417 |
| PGM | FFPE | <100 | 0 | 139 | 0.000 |
| PGM | FFPE | >=100 | 3 | 3 | 1.000 |
| PGM | FF | <100 | 0 | 86 | 0.000 |
| PGM | FF | >=100 | 3 | 3 | 1.000 |

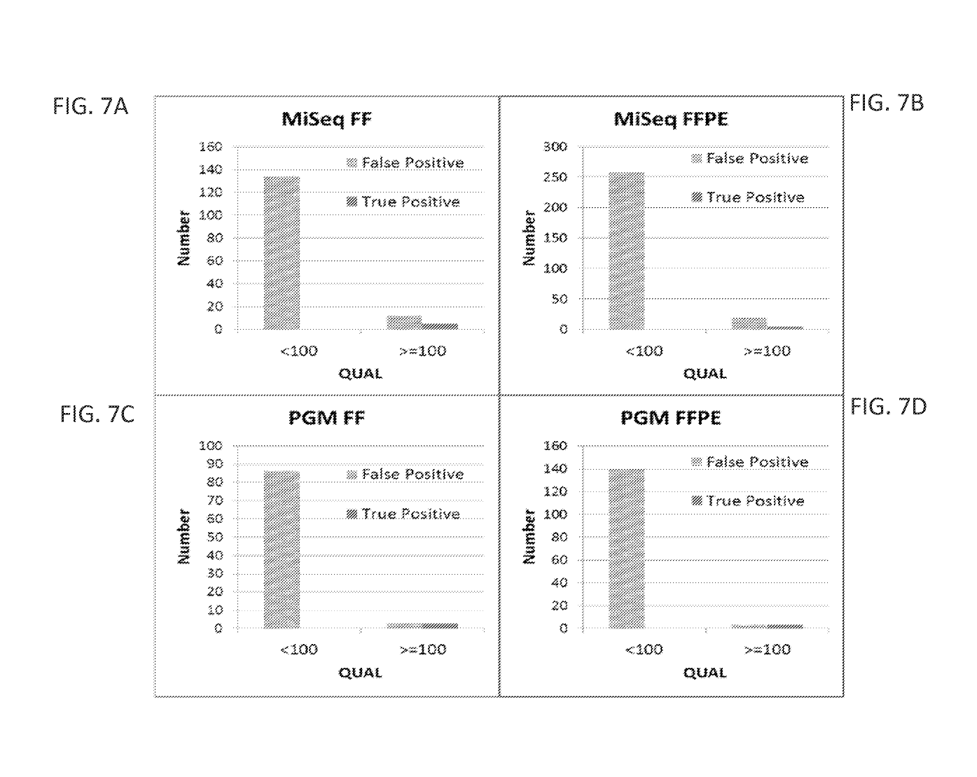

From this analysis it was apparent that using a QUALT of 100 for indel variant calls for PGM FF, PGM FFPE, MiSeq FF, and MiSeq FFPE decreased false positives with no impact to true positives (FIGS. 7A-D), similar to the results for SNV(s). FIGS. 7A-D are graphical representations showing that a QUALT of 100 for indel variant calls for PGM FF, PGM FFPE, MiSeq FF, and MiSeq FFPE decreased false positives with no impact to true positives.

### Systematic Errors (SE)

As a third requirement for calculating the performance characteristics of 20 Gene NGS1, we established a systematic errors (SE) filter for both PGM and MiSeq. SEs were recurrent false positives arbitrarily defined as present in at least one-fourth of all replicates of multiple runs for the Pooled Sample for a specific sequencing platform (PGM or MiSeq) using either FF or FFPE fixation type specimens. There were 20 replicates each of the Pooled Sample for PGM FF and FFPE allowing for any false positive identified in five or more replicates for either fixation type to be classified as a recurrent false positive. There were 16 and 12 replicates each of the Pooled Sample for MiSeq FF and FFPE, respectively, allowing for any false positive identified in 4 and 3 or more replicates, respectively, for either fixation type to be classified as a recurrent false positive. SEs for SNV(s) were common for both PGM and MiSeq (Table 8), and with the exception of one SE (chr10:43614995:SNP:C:T) for the RET gene, were not shared between the two sequencing platforms.

TABLE 8
SNV systematic errors (SE) in the Pooled Sample.

| | Systematic Errors | | | | | | | |
|---|---|---|---|---|---|---|---|---|
| | MiSeq FF | | MiSeq FFPE | | PGM FF | | PGM FFPE | |
| Gene | Unique | Total | Unique | Total | Unique | Total | Unique | Total |
| AKT1 | 1 | 4 | 1 | 3 | 0 | 0 | 0 | 0 |
| ALK | 17 | 116 | 9 | 63 | 0 | 0 | 0 | 0 |
| BRAF | 16 | 64 | 10 | 31 | 0 | 0 | 0 | 0 |
| CTNNB1 | 12 | 48 | 11 | 36 | 0 | 0 | 0 | 0 |
| DDR2 | 13 | 52 | 15 | 48 | 1 | 6 | 0 | 0 |
| EGFR | 17 | 68 | 11 | 34 | 0 | 0 | 0 | 0 |
| ERBB2 | 10 | 52 | 5 | 23 | 1 | 8 | 1 | 13 |
| GNAQ | 5 | 32 | 6 | 26 | 0 | 0 | 0 | 0 |
| JAK2 | 20 | 80 | 18 | 57 | 1 | 20 | 2 | 21 |
| KIT | 18 | 82 | 16 | 58 | 0 | 0 | 0 | 0 |
| KRAS | 4 | 16 | 3 | 9 | 0 | 0 | 0 | 0 |
| MAP2K1 | 5 | 20 | 2 | 6 | 0 | 0 | 0 | 0 |
| NRAS | 3 | 12 | 3 | 11 | 0 | 0 | 0 | 0 |
| PDGFRA | 20 | 80 | 14 | 42 | 0 | 0 | 0 | 0 |
| PIK3CA | 15 | 60 | 17 | 59 | 1 | 5 | 0 | 0 |
| PTEN | 13 | 98 | 6 | 43 | 1 | 13 | 0 | 0 |
| RET | 5 | 26 | 5 | 16 | 1 | 8 | 0 | 0 |
| SMAD4 | 6 | 24 | 6 | 19 | 1 | 8 | 1 | 5 |
| SMO | 7 | 35 | 4 | 16 | 0 | 0 | 0 | 0 |
| Total | 207 | 969 | 162 | 600 | 7 | 68 | 4 | 39 |
| Average per gene | 10.35 | | 8.1 | | 0.35 | | 0.2 | |

The highest number of unique SNV SEs was seen with MiSeq FF for PDGFRA and JAK2, each with a total of 20 (FIG. 8). FIG. 8 is a graphical representation of the numbers of unique SNV systematic errors in the 20-gene validation testing. Other genes with 10 or more SEs in one or more sequencing platforms included PGM FFPE ALK, BRAF, CTNNB1, DDR2, ERBB2, EGFR, KIT, PIK3CA, and PTEN. There was an average of 10.3 SEs per gene for MiSeq FF, followed closely by MiSeq FFPE at 8.1. For PGM FF and FFPE the average number of SEs per gene at 0.35 and 0.2, respectively, was much lower. The highest total number of SNV SEs was seen with MiSeq FF for which there were 207 unique and 969 total, respectively, followed closely by MiSeq FFPE at 162 unique and 600 total. There were fewer SNV SEs for PGM FF (7 unique, 168 total) and FFPE (4 unique, 39 total). For total SNV SEs, almost two-thirds (65.4%) were shared by FF and FFPE for the PGM, while just slightly less than 20% were shared between FF and FFPE for MiSeq. Conversely, the overlap of unique SEs was higher for PGM FF and FFPE (37%) than for MiSeq FF and FFPE (6%) (FIG. 8).

On a variant specific basis, there were 11 variants for MiSeq FF and FFPE present in 75% or more of all replicates, of which six were common to both (Table 9). For MiSeq FF, there were 7 SEs present in all 16 replicates (100%) of the Pooled Sample. For PGM FF and FFPE, there were two variants present in 75% or more of all replicates, of which one was common to both. None of the SNV SEs for either PGM or MiSeq corresponded to actionable variants in the clinical application of 20 Gene NGS1.

TABLE 9
SNV systematic errors identified in at least
75% of Pooled Sample replicates.

| | |
|---|---|
| Gene | TumAltVariant |

MiSeq FFPE

| | |
|---|---|
| ALK | *chr2: 29443611: SNP: T: C |
| ALK | *chr2: 29448427: SNP: T: G |
| ALK | chr2: 29451790: SNP: T: C |
| ALK | chr2: 29451793: SNP: A: C |
| ALK | chr2: 29451799: SNP: T: C |
| ERBB2 | chr17: 37883561: SNP: A: G |
| GNAQ | *chr9: 80430646: SNP: A: C |
| KIT | chr4: 55593476: SNP: T: C |
| PTEN | *chr10: 89692913: SNP: G: A |
| PTEN | *chr10: 89692921: SNP: A: T |
| PTEN | *chr10: 89692923: SNP: G: A |

MiSeq FF

| | |
|---|---|
| ALK | *chr2: 29448427: SNP: T: G |
| ALK | chr2: 29940543: SNP: G: A |
| GNAQ | *chr9: 80430646: SNP: A: C |
| KIT | chr4: 55593476: SNP: T: C |

PGM FF

| | |
|---|---|
| JAK2 | *chr9: 5077517: SNP: T: C |
| PTEN | chr10: 89720683: SNP: C: G |

PGM FFPE

| | |
|---|---|
| JAK2 | *chr9: 5077517: SNP: T: C |
| ERBB2 | chr17: 37872084: SNP: C: T |

SNV systematic errors identified in 100%
of Pooled Sample replicates.
MiSeq FF

| Gene | TumAltVariant |
|---|---|
| ALK | *chr2: 29443611: SNP: T: C |
| ALK | *chr2: 29448423: SNP: T: G |
| ERBB2 | *chr17: 37883561: SNP: A: G |
| GNAQ | *chr9: 80430646: SNP: A: C |
| PTEN | *chr10: 89692913: SNP: G: A |
| PTEN | *chr10: 89692921: SNP: A: T |
| PTEN | *chr10: 89692923: SNP: G: A |

*Systematic errors identified in both FF and FFPE specimen for either MiSeq or PGM.

There were 255 total false positive indels in the multiple replicates of the Paired Samples that represented 101 unique variant calls. While the average number of false positive indels per sample was relatively close for all sequencing platforms and tissue fixation type at 3.6 for Miseq FF, 5.4 for MiSeq FFPE, 3.0 for PGM FF, and 3.6 for PGM FFPE per run, recurrent examples, or SEs, were much more common for MiSeq than for PGM (Table 10).

TABLE 10
Replicates with indel systematic errors (SE) in the Pooled Sample.

| | | | | | |
|---|---|---|---|---|---|
| | | PGM | PGM | MiSeq | MiSeq |
| Gene | TumAltVariant | FF | FFPE | FF | FFPE |

| | | | | |
|---|---|---|---|---|
| AKT1 | chr14: 105242073: INDEL: CTC: — | | 25% | |
| ALK | chr2: 29451783: INDEL: —: A | | | 58% |
| ALK | chr2: 30143052: INDEL: G: — | | | 25% |
| BRAF | chr7: 140482927: INDEL: —: G | | 50% | 25% |
| BRAF | chr7: 140482927: INDEL: G: — | | 100% | 92% |
| CTNNB1 | chr3: 41277987: INDEL: —: A | 30% | | |
| KRAS | chr12: 25368434: INDEL: T: — | | 50% | 42% |
| MAP2K1 | chr15: 66782062: INDEL: A: — | | 63% | 100% |
| SMO | chr7: 128829040: INDEL: GCT: — | | | 33% |

There were no systematic indel errors for PGM FFPE and only one for PGM FF involving the gene CTNNB1, a single base pair insertion —:A at 3:41277987. There were a total of 7 systematic indel errors for MiSeq FFPE and 5 for MiSeq FF for which 4 were common to both groups. The most common systematic indel error identified in all 16 MiSeq FF and 11 of 12 MiSeq FFPE replicates for the Pooled Sample involved the BRAF gene and was a single base pair deletion G:—at 7:140482927. Similar to SNV(s) none of the SEs for indels corresponded to actionable variants in the clinical application of 20 Gene NGS1.

### Minimum Variant Allelic Frequency (MVAF)

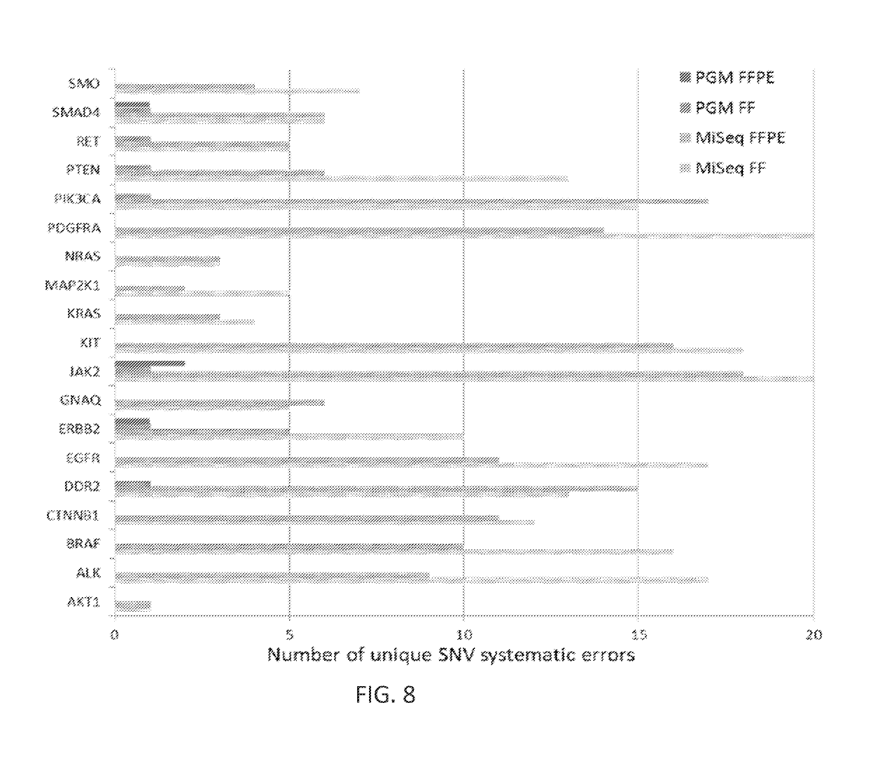

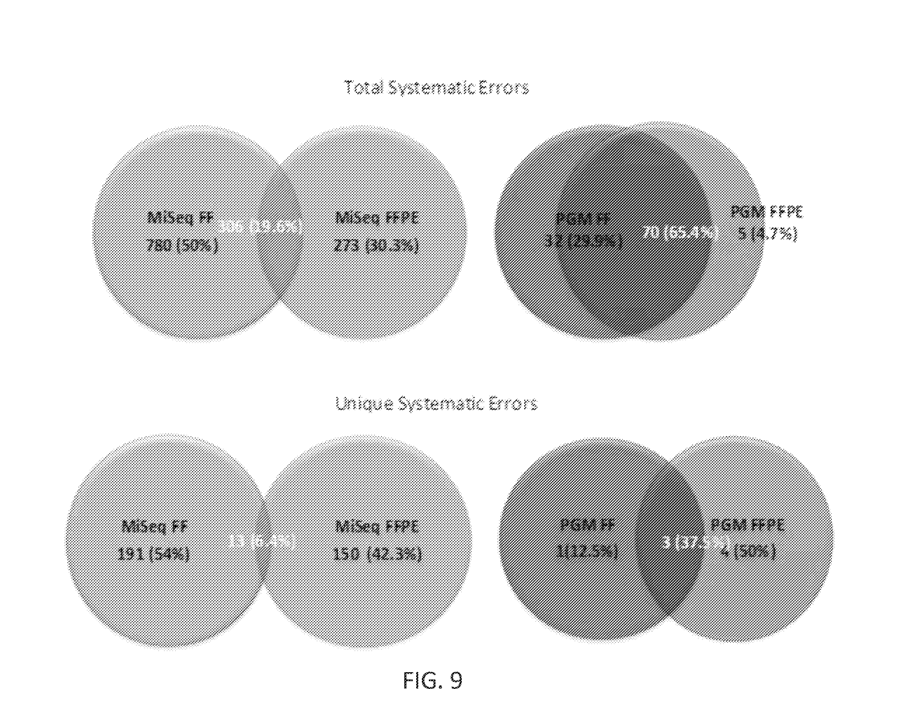

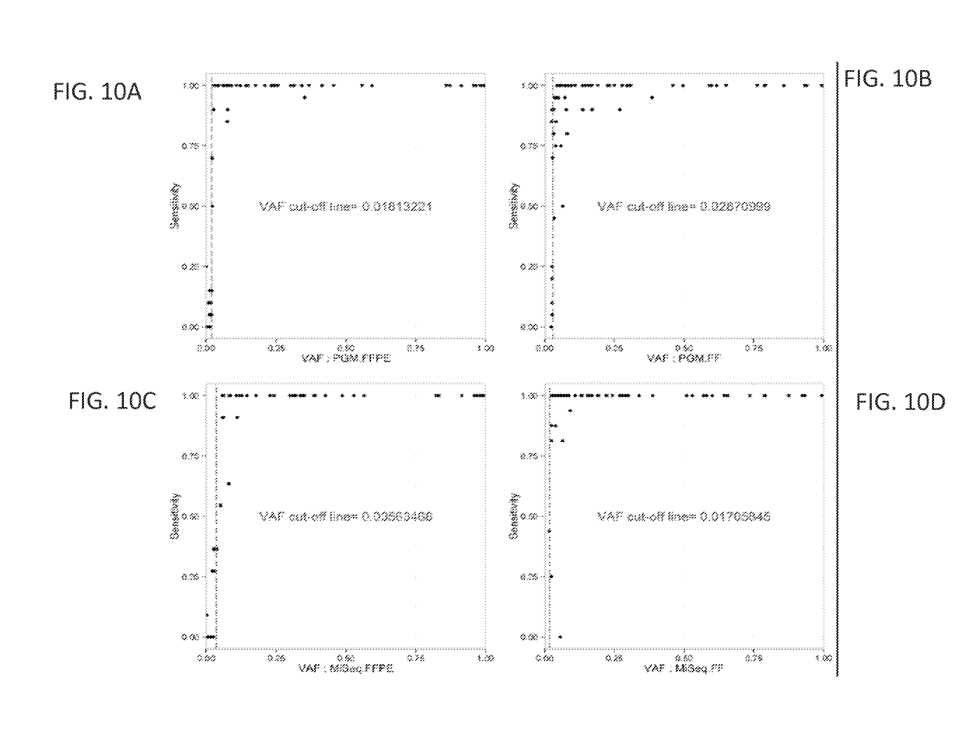

Analytical sensitivity, which is defined by the minimum VAF (MVAF) to achieve estimated 95% overall sensitivity for variants with an equivalent or greater VAF, was calculated using multiple replicates of the Pooled Sample for each sequencing platform and tissue fixation type. There were 16 replicates (libraries) of the Pooled Sample for the MiSeq FF, 12 for MiSeq FFPE, and 20 each for PGM FF and FFPE. To identify the minimum VAF, cumulative sensitivity for a given sequencing platform and specimen type using the Pooled Sample for multiple replicates across multiple runs was calculated for a descending ranking of all variants by VAF and then identifying that variant frequency when sensitivity was below 95%. The results for SNV(s) showed that all platforms for all fixation specimen types achieved 95% sensitivity below our initial targeted goal of a 5% VAF (FIGS. 10A-D, showing MVAF for each sequencing platform and tissue fixation type). As shown in FIGS. 10A-D, the lowest VAF for analytical sensitivity was achieved with MiSeq FF (1.7% VAF), although the value for PGM FFPE at 1.8% VAF was similar and with only minor differences to PGM FF (2.9% VAF) and MiSeq FFPE (3.6% VAF).

Any sample in the Paired Samples with an indel was selected to be in the Pooled Sample, but due to the uniqueness of the 5 indels in this group the predicted VAF for each was very low (Table 11). This issue was further complicated by the lower VAF values for FFPE than for FF specimens due to NA12878 included in the Pooled Sample with none of the 5 indels above a VAF of 4% for PGM FFPE or MiSeq FFPE. Despite the low VAF for all indels, the MiSeq FF results showed 100% sensitivity for all 5 indels with the value for MVAF for indels at 1.8%. There was only one indel at >95% sensitivity for MiSeq FFPE with a VAF of 3.5% which represented the MVAF in this instance. PGM FF and PGM FFPE failed to detect any of the 5 indels, thus not defining a definitive MVAF from the Pooled Sample.

TABLE 11
Indel detection for multiple replicates of the Pooled Sample using ITVC
v3.6.63335 (PGM) and SVC v.2.1.12 (MiSeq).

| | | PGM FF | | PGM FFPE | |
|---|---|---|---|---|---|
| | | Percent | Predicted | Percent | Predicted |
| Gene | Variant | detected | VAF | detected | VAF |
| CTNNB1 | 3:41266133:Indel:CCTT:C | 0% | 0.0253988 | 0% | 4.266E-21 |
| PTEN | 10:89717727:Indel: | 0% | 0.0189221 | 0% | 0.0071379 |
| | GTGATATCAAA:- | | | | |
| PTEN | 10:89692825:Indel:CT:C | 0% | 0.0341489 | 0% | 0.0088217 |
| PTEN | 10:89717769:Indel:TA:T | 5% | 0.0396813 | 10% | 0.0366132 |
| SMAD4 | 18:48584513:Indel:TG:T | 0% | 0.1080947 | 0% | 0.0240309 |
| | | MiSeq FF | | MiSeq FFPE | |
| | | Percent | Predicted | Percent | Predicted |
| Gene | Variant | detected | VAF | detected | VAF |
| CTNNB1 | 3:41266133:Indel:CCTT:C | 100% | 0.0290688 | 0% | 4.421E-21 |
| PTEN | 10:89717727:Indel: | 100% | 0.0189221 | 36% | 0.0072934 |
| | GTGATATCAAA:- | | | | |
| PTEN | 10:89692825:Indel:CT:C | 100% | 0.0411537 | 9% | 0.0095129 |
| PTEN | 10:89717769:Indel:TA:T | 100% | 0.042278 | 100% | 0.0366132 |
| SMAD4 | 18:48584513:Indel:TG:T | 100% | 0.1080947 | 9% | 0.0240309 |

Given the inability to define a MVAF for indels for PGM FF and PGM FFPE using the Pooled Sample, we utilized an additional cohort, the Paired Samples, to define this value. To briefly summarize the results in the Paired Samples, three of five known unique indels ranging from a VAF of 25% to 55% were detected for both PGM FF and FFPE. We thus currently define the MVAF in indels for PGM FF and FFPE at a VAF of 25%, but with 60% confidence of detection of such variants.

### Summary of Thresholds and Filters

Tables 12 and 13 summarize the threshold, trend values and filters applied at the run, sample, amplicon or base pair level identified in the validation of 20 Gene NGS1 that was applied as part of the clinical standard operating procedure. Run and sample threshold and trend values are displayed and managed via an information management system. Base pair level thresholds and filters are applied as per the techniques set forth herein.

TABLE 12
PGM thresholds and filters.

| | | | FF Threshold or | FFPE Threshold |
|---|---|---|---|---|
| Level | Threshold | Unit | Trend Value | or Trend Value |
| Run | Total bases | total bases in M | 274 | 274 |
| Run | Key signal | value | 63 | 63 |
| Run | Total Reads | number reads in | 299 | 299 |
| | | M | | |
| Run | mean read length | number of base | 90 | 90 |
| | | pairs | | |
| Run | % aligned bases | percent base pairs | 95 | 95 |
| Run | Total number of | total bases in M | 162 | 162 |
| | bases AQ20 | | | |
| Run | Mapped Reads | number reads | 44493 | 44493 |
| | (No template | | | |
| | control) | | | |
| Run | Positive | Percent | 95 | 95 |
| | Sensitivity | | | |
| | Control | | | |
| Sample | Mapped Reads | number reads | 157,672 | 157,672 |
| Sample | Mean Depth | Number bases | 174 | 174 |
| Sample | On Target | Percent | 80 | 80 |
| Sample | Uniformity | Percent | 76 | 76 |
| Base pair | MVRT for | number reads | <20 | <21 |
| | SNV(s) | | | |
| Base pair | QUALT for | value | >100 | >100 |
| | SNV(s) & indels | | | |
| Base pair | SE for SNV(s) & | See list | See Tables 14A | See Tables 14A |
| | indels | | and 14B | and 14B |
| Base pair | MVAF for | percent variant | 2.8709985% | 1.8132212% |
| | SNV(s) | reads | | |

TABLE 13
MiSeq thresholds and filters.

| | Summary Report | | FF Threshold | FFPE Threshold |
|---|---|---|---|---|
| Level | Field Name | Unit | or Trend Value | or Trend Value |
| Run | Density (K/mm2) | value | 241 | 241 |
| Run | Clusters PF (%) | value | 85 | 85 |
| Run | ReadsPF (M) | number reads in | 5 | 5 |
| | | M | | |
| Run | % >= Q30 | percent base pairs | 96 | 96 |
| Run | Positive Sensitivity | Percent | 95 | 95 |
| | Control | | | |
| Sample | Clusters PF | value | 135,119 | 135,119 |
| Sample | Coverage | Number bases | 250 | 250 |
| Base pair | MVRT for SNV(s) | number reads | <5 | <10 |
| Base pair | QUALT for | value | >100 | >100 |
| | SNV(s) & indels | | | |
| Base pair | SE for SNV(s) & | See list | See Tables 14A | See Tables 14A |
| | indels | | and 14B | and 14B |
| Base pair | MVAF for SNV(s) | percent variant | 1.7058452% | 3.5634685% |
| | | reads | | |
| Base pair | MVAF for indels | percent variant | 1.8922108% | 3.6613217% |
| | | reads | | |

TABLE 14A
SNV systematic error (SE) frequency counts in the Pooled Sample.

| | | MiSeq | MiSeq | PGM | PGM |
|---|---|---|---|---|---|
| Gene | TumAltVariant | FF | FFPE | FF | FFPE |
| ALK | chr2: 29443611: SNP: T: C | 16 | 11 | | |
| ALK | chr2: 29448423: SNP: T: G | 16 | 7 | | |
| ERBB2 | chr17: 37883561: SNP: A: G | 16 | 11 | | |
| GNAQ | chr9: 80430646: SNP: A: C | 16 | 11 | | |
| PTEN | chr10: 89692913: SNP: G: A | 16 | 11 | | |
| PTEN | chr10: 89692921: SNP: A: T | 16 | 11 | | |
| PTEN | chr10: 89692923: SNP: G: A | 16 | 11 | | |
| ALK | chr2: 29448427: SNP: T: G | 14 | 10 | | |
| ALK | chr2: 29940543: SNP: G: A | 14 | | | |
| KIT | chr4: 55593476: SNP: T: C | 14 | 11 | | |
| PTEN | chr10: 89692993: SNP: G: A | 14 | | | |
| SMO | chr7: 128829066: SNP: A: G | 11 | 7 | | |
| ALK | chr2: 29451790: SNP: T: C | 10 | 8 | | |
| RET | chr10: 43614995: SNP: C: T | 10 | | 8 | |
| ALK | chr2: 29451793: SNP: A: C | 9 | 9 | | |
| ALK | chr2: 29451799: SNP: T: C | 9 | 9 | | |
| AKT1 | chr14: 105239610: SNP: G: A | 4 | | | |
| ALK | chr2: 29416517: SNP: G: A | 4 | | | |
| ALK | chr2: 29419683: SNP: T: C | 4 | | | |
| ALK | chr2: 29432701: SNP: C: T | 4 | | | |
| ALK | chr2: 29450489: SNP: C: T | 4 | | | |
| ALK | chr2: 29606659: SNP: G: A | 4 | | | |
| ALK | chr2: 29917833: SNP: G: A | 4 | | | |
| ALK | chr2: 29940498: SNP: G: A | 4 | | | |
| BRAF | chr7: 140449097: SNP: T: C | 4 | | | |
| BRAF | chr7: 140449155: SNP: A: G | 4 | | | |
| BRAF | chr7: 140449200: SNP: T: A | 4 | | | |
| BRAF | chr7: 140453167: SNP: C: T | 4 | | | |
| BRAF | chr7: 140476731: SNP: G: A | 4 | | | |
| BRAF | chr7: 140476776: SNP: T: A | 4 | | | |
| BRAF | chr7: 140494225: SNP: T: A | 4 | | | |
| BRAF | chr7: 140500211: SNP: C: T | 4 | | | |
| BRAF | chr7: 140500256: SNP: C: T | 4 | | | |
| BRAF | chr7: 140501324: SNP: A: G | 4 | | | |
| BRAF | chr7: 140507806: SNP: A: G | 4 | | | |
| BRAF | chr7: 140507851: SNP: G: A | 4 | | | |
| BRAF | chr7: 140508739: SNP: C: T | 4 | | | |
| BRAF | chr7: 140508784: SNP: C: T | 4 | | | |
| BRAF | chr7: 140534591: SNP: C: T | 4 | | | |
| BRAF | chr7: 140534652: SNP: G: A | 4 | | | |
| CTNNB1 | chr3: 41266078: SNP: G: A | 4 | | | |
| CTNNB1 | chr3: 41266180: SNP: C: T | 4 | | | |
| CTNNB1 | chr3: 41266225: SNP: C: T | 4 | | | |
| CTNNB1 | chr3: 41266570: SNP: C: T | 4 | | | |
| CTNNB1 | chr3: 41266944: SNP: A: T | 4 | | | |
| CTNNB1 | chr3: 41266989: SNP: C: T | 4 | | | |
| CTNNB1 | chr3: 41267280: SNP: A: G | 4 | | | |
| CTNNB1 | chr3: 41275070: SNP: T: A | 4 | | | |
| CTNNB1 | chr3: 41275686: SNP: G: A | 4 | | | |
| CTNNB1 | chr3: 41277262: SNP: T: C | 4 | | | |
| CTNNB1 | chr3: 41277891: SNP: T: A | 4 | | | |
| CTNNB1 | chr3: 41277965: SNP: A: T | 4 | | | |
| DDR2 | chr1: l 62722928: SNP: A: T | 4 | | | |
| DDR2 | chr1: 162724486: SNP: T: A | 4 | | | |
| DDR2 | chr1: 162725045: SNP: T: A | 4 | | | |
| DDR2 | chr1: 162731013: SNP: A: G | 4 | | | |
| DDR2 | chr1: 162737068: SNP: C: T | 4 | | | |
| DDR2 | chr1: 162737113: SNP: C: T | 4 | | | |
| DDR2 | chr1: 162740148: SNP: T: A | 4 | | | |
| DDR2 | chr1: 162745494: SNP: A: G | 4 | | | |
| DDR2 | chr1: 162746097: SNP: T: C | 4 | | | |
| DDR2 | chr1: 162746142: SNP: T: C | 4 | | | |
| DDR2 | chr1: 162748421: SNP: G: A | 4 | | | |
| DDR2 | chr1: 162749947: SNP: C: T | 4 | | | |
| DDR2 | chr1: 162749992: SNP: T: C | 4 | | | |
| EGFR | chr7: 55210128: SNP: A: T | 4 | | | |
| EGFR | chr7: 55211063: SNP: A: G | 4 | | | |
| EGFR | chr7: 55211163: SNP: C: T | 4 | | | |
| EGFR | chr7: 55221736: SNP: C: T | 4 | | | |
| EGFR | chr7: 55224240: SNP: G: A | 4 | | | |
| EGFR | chr7: 55224512: SNP: A: G | 4 | | | |
| EGFR | chr7: 55225405: SNP: C: T | 4 | | | |
| EGFR | chr7: 55227913: SNP: T: A | 4 | | | |
| EGFR | chr7: 55228015: SNP: A: T | 4 | | | |
| EGFR | chr7: 55233081: SNP: G: A | 4 | | | |
| EGFR | chr7: 55241618: SNP: T: C | 4 | | | |
| EGFR | chr7: 55242462: SNP: C: T | 4 | | | |
| EGFR | chr7: 55242507: SNP: C: T | 4 | | | |
| EGFR | chr7: 55259475: SNP: G: A | 4 | | | |
| EGFR | chr7: 55260512: SNP: C: T | 4 | | | |
| EGFR | chr7: 55268042: SNP: T: C | 4 | | | |
| EGFR | chr7: 55270306: SNP: C: T | 4 | | | |
| ERBB2 | chr17: 37863243: SNP: T: C | 4 | | | |
| ERBB2 | chr17: 37866664: SNP: C: T | 4 | | | |
| ERBB2 | chr17: 37866709: SNP: C: T | 4 | | | |
| ERBB2 | chr17: 37868665: SNP: T: A | 4 | | | |
| ERBB2 | chr17: 37872162: SNP: C: T | 4 | | | |
| ERBB2 | chr17: 37879900: SNP: G: A | 4 | | | |
| ERBB2 | chr17: 37880214: SNP: A: T | 4 | | | |
| ERBB2 | chr17: 37883223: SNP: G: A | 4 | | | |
| ERBB2 | chr17: 37884125: SNP: C: T | 4 | | | |
| GNAQ | chr9: 80336272: SNP: G: A | 4 | | | |
| GNAQ | chr9: 80336317: SNP: G: A | 4 | | | |
| GNAQ | chr9: 80343470: SNP: G: A | 4 | | | |
| GNAQ | chr9: 80537131: SNP: G: A | 4 | | | |
| JAK2 | chr9: 5029832: SNP: A: G | 4 | | | |
| JAK2 | chr9: 5029877: SNP: A: G | 4 | | | |
| JAK2 | chr9: 5044497: SNP: G: A | 4 | | | |
| JAK2 | chr9: 5050743: SNP: G: A | 4 | | | |
| JAK2 | chr9: 5050788: SNP: A: G | 4 | | | |
| JAK2 | chr9: 5054740: SNP: C: T | 4 | | | |
| JAK2 | chr9: 5054785: SNP: T: C | 4 | | | |
| JAK2 | chr9: 5054849: SNP: G: A | 4 | | | |
| JAK2 | chr9: 5055693: SNP: C: T | 4 | | | |
| JAK2 | chr9: 5064937: SNP: G: A | 4 | | | |
| JAK2 | chr9: 5066689: SNP: C: T | 4 | | | |
| JAK2 | chr9: 5070032: SNP: A: G | 4 | | | |
| JAK2 | chr9: 5073735: SNP: C: T | 4 | | | |
| JAK2 | chr9: 5080275: SNP: T: A | 4 | | | |
| JAK2 | chr9: 5080370: SNP: A: T | 4 | | | |
| JAK2 | chr9: 5080570: SNP: C: T | 4 | | | |
| JAK2 | chr9: 5081823: SNP: G: A | 4 | | | |
| JAK2 | chr9: 5090479: SNP: T: C | 4 | | | |
| JAK2 | chr9: 5090803: SNP: T: A | 4 | | | |
| JAK2 | chr9: 5126717: SNP: A: T | 4 | | | |
| KIT | chr4: 55561735: SNP: G: A | 4 | | | |
| KIT | chr4: 55564601: SNP: T: C | 4 | | | |
| KIT | chr4: 55564646: SNP: C: T | 4 | | | |
| KIT | chr4: 55564697: SNP: G: A | 4 | | | |
| KIT | chr4: 55565890: SNP: G: A | 4 | | | |
| KIT | chr4: 55569957: SNP: T: A | 4 | | | |
| KIT | chr4: 55573314: SNP: A: G | 4 | | | |
| KIT | chr4: 55575695: SNP: T: A | 4 | | | |
| KIT | chr4: 55589797: SNP: C: T | 4 | | | |
| KIT | chr4: 55589842: SNP: T: C | 4 | | | |
| KIT | chr4: 55592178: SNP: C: T | 4 | | | |
| KIT | chr4: 55593440: SNP: G: A | 4 | | | |
| KIT | chr4: 55593617: SNP: G: A | 4 | | | |
| KIT | chr4: 55593662: SNP: T: A | 4 | | | |
| KIT | chr4: 55595574: SNP: A: G | 4 | | | |
| KIT | chr4: 55595619: SNP: T: A | 4 | | | |
| KIT | chr4: 55598128: SNP: G: A | 4 | | | |
| KRAS | chr12: 25362835: SNP: T: A | 4 | | | |
| KRAS | chr12: 25368425: SNP: C: T | 4 | | | |
| KRAS | chr12: 25378570: SNP: T: C | 4 | | | |
| KRAS | chr12: 25380304: SNP: G: A | 4 | | | |
| MAP2K1 | chr15: 66727385: SNP: A: G | 4 | | | |
| MAP2K1 | chr15: 66727430: SNP: G: A | 4 | | | |
| MAP2K1 | chr15: 66774140: SNP: C: T | 4 | | | |
| MAP2K1 | chr15: 66777493: SNP: C: T | 4 | | | |
| MAP2K1 | chr15: 66782887: SNP: A: T | 4 | | | |
| NRAS | chr1: 115251251: SNP: G: A | 4 | | | |
| NRAS | chr1: 115258722: SNP: T: C | 4 | | | |
| NRAS | chr1: 115258767: SNP: T: C | 4 | | | |
| PDGFRA | chr4: 55127319: SNP: A: G | 4 | | | |
| PDGFRA | chr4: 55127499: SNP: G: A | 4 | | | |
| PDGFRA | chr4: 55129869: SNP: G: A | 4 | | | |
| PDGFRA | chr4: 55131135: SNP: G: A | 4 | | | |
| PDGFRA | chr4: 55131180: SNP: G: A | 4 | | | |
| PDGFRA | chr4: 55133791: SNP: T: C | 4 | | | |
| PDGFRA | chr4: 55133836: SNP: T: C | 4 | | | |
| PDGFRA | chr4: 55136843: SNP: A: T | 4 | | | |
| PDGFRA | chr4: 55141100: SNP: T: A | 4 | | | |
| PDGFRA | chr4: 55143619: SNP: G: A | 4 | | | |
| PDGFRA | chr4: 55144610: SNP: G: A | 4 | | | |
| PDGFRA | chr4: 55146511: SNP: G: A | 4 | | | |
| PDGFRA | chr4: 55151562: SNP: C: T | 4 | | | |
| PDGFRA | chr4: 55151609: SNP: A: G | 4 | | | |
| PDGFRA | chr4: 55155023: SNP: G: A | 4 | | | |
| PDGFRA | chr4: 55155266: SNP: T: A | 4 | | | |
| PDGFRA | chr4: 55156537: SNP: A: G | 4 | | | |
| PDGFRA | chr4: 55156582: SNP: A: G | 4 | | | |
| PDGFRA | chr4: 55161364: SNP: G: A | 4 | | | |
| PDGFRA | chr4: 55161409: SNP: T: A | 4 | | | |
| PIK3CA | chr3: 178916864: SNP: A: G | 4 | | | |
| PIK3CA | chr3: 178917554: SNP: T: A | 4 | | | |
| PIK3CA | chr3: 178919217: SNP: A: T | 4 | | | |
| PIK3CA | chr3: 178921361: SNP: G: A | 4 | | | |
| PIK3CA | chr3: 178927478: SNP: G: A | 4 | | | |
| PIK3CA | chr3: 178928059: SNP: G: A | 4 | | | |
| PIK3CA | chr3: 178937027: SNP: C: T | 4 | | | |
| PIK3CA | chr3: 178937383: SNP: C: T | 4 | | | |
| PIK3CA | chr3: 178942574: SNP: T: A | 4 | | | |
| PIK3CA | chr3: 178943789: SNP: T: C | 4 | | | |
| PIK3CA | chr3: 178947115: SNP: G: A | 4 | | | |
| PIK3CA | chr3: 178947839: SNP: G: A | 4 | | | |
| PIK3CA | chr3: 178947884: SNP: A: G | 4 | | | |
| PIK3CA | chr3: 178948018: SNP: T: C | 4 | | | |
| PIK3CA | chr3: 178948126: SNP: A: G | 4 | | | |
| PTEN | chr10: 89624266: SNP: A: T | 4 | | | |
| PTEN | chr10: 89653845: SNP: A: G | 4 | | | |
| PTEN | chr10: 89711982: SNP: T: A | 4 | | | |
| PTEN | chr10: 89717664: SNP: G: A | 4 | | | |
| PTEN | chr10: 89717765: SNP: A: T | 4 | | | |
| PTEN | chr10: 89720720: SNP: G: A | 4 | | | |
| PTEN | chr10: 89720765: SNP: A: G | 4 | | | |
| PTEN | chr10: 89720850: SNP: A: T | 4 | | | |
| PTEN | chr10: 89725199: SNP: A: T | 4 | | | |
| RET | chr10: 43597929: SNP: C: T | 4 | | | |
| RET | chr10: 43597974: SNP: C: T | 4 | | | |
| RET | chr10: 43617433: SNP: T: C | 4 | | | |
| RET | chr10: 43623579: SNP: G: A | 4 | | | |
| SMAD4 | chr18: 48573550: SNP: A: T | 4 | | | |
| SMAD4 | chr18: 48573595: SNP: C: T | 4 | | | |
| SMAD4 | chr18: 48575219: SNP: C: T | 4 | | | |
| SMAD4 | chr18: 48586247: SNP: A: T | 4 | | | |
| SMAD4 | chr18: 48591847: SNP: A: G | 4 | | | |
| SMAD4 | chr18: 48593467: SNP: G: A | 4 | | | |
| SMO | chr7: 128829251: SNP: G: A | 4 | | | |
| SMO | chr7: 128829296: SNP: G: A | 4 | | | |
| SMO | chr7: 128843232: SNP: G: A | 4 | | | |
| SMO | chr7: 128846053: SNP: G: A | 4 | | | |
| SMO | chr7: 128851524: SNP: G: A | 4 | | | |
| SMO | chr7: 128851569: SNP: A: G | 4 | | | |
| AKT1 | chr14: 105239336: SNP: T: C | | 3 | | |
| ALK | chr2: 29416457: SNP: G: A | | 3 | | |
| ALK | chr2: 29416526: SNP: A: T | | 3 | | |
| ALK | chr2: 29606653: SNP: A: T | | 3 | | |
| BRAF | chr7: 140434472: SNP: A: G | | 3 | | |
| BRAF | chr7: 140453162: SNP: T: A | | 3 | | |
| BRAF | chr7: 140476739: SNP: A: T | | 3 | | |
| BRAF | chr7: 140481419: SNP: A: T | | 3 | | |
| BRAF | chr7: 140494113: SNP: T: A | | 3 | | |
| BRAF | chr7: 140501271: SNP: T: C | | 3 | | |
| BRAF | chr7: 140507805: SNP: C: T | | 3 | | |
| BRAF | chr7: 140508710: SNP: A: G | | 4 | | |
| BRAF | chr7: 140534580: SNP: A: G | | 3 | | |
| BRAF | chr7: 140549976: SNP: C: T | | 3 | | |
| CTNNB1 | chr3: 41266151: SNP: G: A | | 4 | | |
| CTNNB1 | chr3: 41266223: SNP: T: A | | 3 | | |
| CTNNB1 | chr3: 41266466: SNP: T: C | | 3 | | |
| CTNNB1 | chr3: 41267211: SNP: T: A | | 3 | | |
| CTNNB1 | chr3: 41268744: SNP: A: T | | 3 | | |
| CTNNB1 | chr3: 41274855: SNP: C: T | | 3 | | |
| CTNNB1 | chr3: 41275037: SNP: C: T | | 3 | | |
| CTNNB1 | chr3: 41277312: SNP: A: G | | 3 | | |
| CTNNB1 | chr3: 41277857: SNP: T: C | | 3 | | |
| CTNNB1 | chr3: 41279525: SNP: G: A | | 3 | | |
| CTNNB1 | chr3: 41280641: SNP: T: C | | 5 | | |
| DDR2 | chr1: 162688874: SNP: G: A | | 4 | | |
| DDR2 | chr1: 162722964: SNP: G: A | | 3 | | |
| DDR2 | chr1: 162724515: SNP: T: C | | 3 | | |
| DDR2 | chr1: 162724997: SNP: C: T | | 3 | | |
| DDR2 | chr1: 162725549: SNP: G: A | | 3 | | |
| DDR2 | chr1: 162729611: SNP: A: T | | 3 | | |
| DDR2 | chr1: 162729673: SNP: C: T | | 4 | | |
| DDR2 | chr1: 162729722: SNP: G: A | | 3 | | |
| DDR2 | chr1: 162731172: SNP: G: A | | 3 | | |
| DDR2 | chr1: 162735842: SNP: C: A | | | 6 | |
| DDR2 | chr1: 162737039: SNP: G: A | | 3 | | |
| DDR2 | chr1: 162740177: SNP: C: T | | 3 | | |
| DDR2 | chr1: 162745953: SNP: C: T | | 3 | | |
| DDR2 | chr1: 162746145: SNP: G: A | | 3 | | |
| DDR2 | chr1: 162748450: SNP: C: T | | 4 | | |
| DDR2 | chr1: 162750000: SNP: A: G | | 3 | | |
| EGFR | chr7: 55211029: SNP: T: C | | 3 | | |
| EGFR | chr7: 55221811: SNP: C: T | | 3 | | |
| EGFR | chr7: 55224251: SNP: A: T | | 3 | | |
| EGFR | chr7: 55224461: SNP: C: T | | 3 | | |
| EGFR | chr7: 55231512: SNP: G: A | | 3 | | |
| EGFR | chr7: 55233040: SNP: C: T | | 3 | | |
| EGFR | chr7: 55238202: SNP: G: A | | 3 | | |
| EGFR | chr7: 55241681: SNP: C: T | | 3 | | |
| EGFR | chr7: 55260483: SNP: G: A | | 4 | | |
| EGFR | chr7: 55268895: SNP: G: A | | 3 | | |
| EGFR | chr7: 55273176: SNP: C: T | | 3 | | |
| ERBB2 | chr17: 37868630: SNP: C: T | | 3 | | |
| ERBB2 | chr17: 37872084: SNP: C: T | | | 8 | 13 |
| ERBB2 | chr17: 37880251: SNP: A: G | | 3 | | |
| ERBB2 | chr17: 37881151: SNP: T: A | | 3 | | |
| ERBB2 | chr17: 37881606: SNP: G: A | | 3 | | |
| GNAQ | chr9: 80409394: SNP: C: T | | 3 | | |
| GNAQ | chr9: 80412533: SNP: G: A | | 3 | | |
| GNAQ | chr9: 80430543: SNP: G: A | | 3 | | |
| GNAQ | chr9: 80430605: SNP: A: T | | 3 | | |
| GNAQ | chr9: 80537098: SNP: T: C | | 3 | | |
| JAK2 | chr9: 5022082: SNP: T: A | | 3 | | |
| JAK2 | chr9: 5050817: SNP: C: T | | 4 | | |
| JAK2 | chr9: 5054612: SNP: C: T | | 3 | | |
| JAK2 | chr9: 5054780: SNP: G: A | | 3 | | |
| JAK2 | chr9: 5054844: SNP: C: T | | 3 | | |
| JAK2 | chr9: 5064966: SNP: T: C | | 4 | | |
| JAK2 | chr9: 5064981: SNP: A: G | | 3 | | |
| JAK2 | chr9: 5066776: SNP: C: T | | | | 5 |
| JAK2 | chr9: 5069071: SNP: A: G | | 3 | | |
| JAK2 | chr9: 5069135: SNP: T: C | | 3 | | |
| JAK2 | chr9: 5077517: SNP: T: C | | | 20 | 16 |
| JAK2 | chr9: 5080604: SNP: T: A | | 3 | | |
| JAK2 | chr9: 5080666: SNP: A: T | | 3 | | |
| JAK2 | chr9: 5081820: SNP: T: C | | 3 | | |
| JAK2 | chr9: 5089749: SNP: A: G | | 3 | | |
| JAK2 | chr9: 5089813: SNP: C: T | | 3 | | |
| JAK2 | chr9: 5090512: SNP: A: G | | 3 | | |
| JAK2 | chr9: 5123079: SNP: T: C | | 3 | | |
| JAK2 | chr9: 5126386: SNP: T: C | | 3 | | |
| JAK2 | chr9: 5126688: SNP: A: G | | 4 | | |
| KIT | chr4: 55561701: SNP: C: T | | 3 | | |
| KIT | chr4: 55561929: SNP: A: T | | 3 | | |
| KIT | chr4: 55564637: SNP: G: A | | 3 | | |
| KIT | chr4: 55565843: SNP: C: T | | 3 | | |
| KIT | chr4: 55569923: SNP: C: T | | 3 | | |
| KIT | chr4: 55573388: SNP: T: C | | 4 | | |
| KIT | chr4: 55575645: SNP: A: T | | 3 | | |
| KIT | chr4: 55594012: SNP: T: C | | 3 | | |
| KIT | chr4: 55595523: SNP: A: G | | 3 | | |
| KIT | chr4: 55595584: SNP: T: C | | 3 | | |
| KIT | chr4: 55598157: SNP: C: T | | 3 | | |
| KIT | chr4: 55599308: SNP: G: A | | 3 | | |
| KIT | chr4: 55602679: SNP: A: T | | 3 | | |
| KIT | chr4: 55603361: SNP: G: A | | 4 | | |
| KIT | chr4: 55603431: SNP: A: G | | 3 | | |
| KRAS | chr12: 25362791: SNP: T: C | | 3 | | |
| KRAS | chr12: 25368477: SNP: A: G | | 3 | | |
| KRAS | chr12: 25378566: SNP: T: A | | 3 | | |
| MAP2K1 | chr15: 66729101: SNP: C: T | | 3 | | |
| MAP2K1 | chr15: 66777347: SNP: C: T | | 3 | | |
| NRAS | chr1: 115252206: SNP: G: A | | 3 | | |
| NRAS | chr1: 115256446: SNP: A: T | | 4 | | |
| NRAS | chr1: 115256508: SNP: C: T | | 4 | | |
| PDGFRA | chr4: 55127285: SNP: C: T | | 3 | | |
| PDGFRA | chr4: 55129881: SNP: A: T | | 3 | | |
| PDGFRA | chr4: 55130021: SNP: G: A | | 3 | | |
| PDGFRA | chr4: 55131137: SNP: C: T | | 3 | | |
| PDGFRA | chr4: 55133473: SNP: A: T | | 3 | | |
| PDGFRA | chr4: 55136893: SNP: T: A | | 3 | | |
| PDGFRA | chr4: 55143622: SNP: C: T | | 3 | | |
| PDGFRA | chr4: 55144146: SNP: A: G | | 3 | | |
| PDGFRA | chr4: 55144549: SNP: G: A | | 3 | | |
| PDGFRA | chr4: 55144614: SNP: C: T | | 3 | | |
| PDGFRA | chr4: 55146516: SNP: C: T | | 3 | | |
| PDGFRA | chr4: 55151552: SNP: A: G | | 3 | | |
| PDGFRA | chr4: 55151611: SNP: C: T | | 3 | | |
| PDGFRA | chr4: 55153617: SNP: G: A | | 3 | | |
| PIK3CA | chr3: 178916831: SNP: T: C | | 3 | | |
| PIK3CA | chr3: 178917478: SNP: G: A | | 3 | | |
| PIK3CA | chr3: 178917521: SNP: A: T | | 3 | | |
| PIK3CA | chr3: 178917569: SNP: A: G | | 5 | | |
| PIK3CA | chr3: 178919109: SNP: T: C | | 3 | | |
| PIK3CA | chr3: 178921435: SNP: C: T | | 4 | | |
| PIK3CA | chr3: 178921476: SNP: G: A | | 4 | | |
| PIK3CA | chr3: 178927436: SNP: C: T | | 3 | | |
| PIK3CA | chr3: 178927988: SNP: G: A | | 3 | | |
| PIK3CA | chr3: 178928223: SNP: C: T | | 3 | | |
| PIK3CA | chr3: 178936021: SNP: T: A | | 3 | | |
| PIK3CA | chr3: 178936085: SNP: A: T | | 3 | | |
| PIK3CA | chr3: 178937478: SNP: T: A | | | 5 | |
| PIK3CA | chr3: 178938778: SNP: G: A | | 3 | | |
| PIK3CA | chr3: 178938817: SNP: C: T | | 3 | | |
| PIK3CA | chr3: 178942535: SNP: T: C | | 3 | | |
| PIK3CA | chr3: 178947831: SNP: T: C | | 3 | | |
| PIK3CA | chr3: 178948087: SNP: A: T | | 3 | | |
| PIK3CA | chr3: 178951977: SNP: C: T | | 4 | | |
| PTEN | chr10: 89712011: SNP: C: T | | 3 | | |
| PTEN | chr10: 89717736: SNP: A: G | | 4 | | |
| PTEN | chr10: 89720683: SNP: C: G | | | 13 | |
| PTEN | chr10: 89720712: SNP: A: T | | 3 | | |
| RET | chr10: 43597962: SNP: A: G | | 3 | | |
| RET | chr10: 43601985: SNP: C: T | | 3 | | |
| RET | chr10: 43612085: SNP: A: T | | 3 | | |
| RET | chr10: 43622171: SNP: G: A | | 4 | | |
| RET | chr10: 43623696: SNP: A: G | | 3 | | |
| SMAD4 | chr18: 48573584: SNP: T: A | | 3 | | |
| SMAD4 | chr18: 48573644: SNP: A: T | | 3 | | |
| SMAD4 | chr18: 48575190: SNP: G: A | | 4 | | |
| SMAD4 | chr18: 48584714: SNP: C: T | | 3 | | |
| SMAD4 | chr18: 48584803: SNP: T: C | | | 8 | 5 |
| SMAD4 | chr18: 48591814: SNP: T: C | | 3 | | |
| SMAD4 | chr18: 48604832: SNP: G: A | | 3 | | |
| SMO | chr7: 128845486: SNP: C: T | | 3 | | |
| SMO | chr7: 128851536: SNP: C: T | | 3 | | |
| SMO | chr7: 128852049: SNP: G: A | | 3 | | |

TABLE 14B
Indel systematic error (SE) frequency counts in the Pooled Sample.

| | | PGM | PGM | MiSeq | MiSeq |
|---|---|---|---|---|---|
| Gene | Variant | FFPE | FF | FFPE | FF |
| DDR2 | chr1: 162724430: INDEL: G: — | 1 | | | |
| RET | chr10: 43572750: INDEL: TGC: — | | | 1 | 1 |
| RET | chr10: 43600424: INDEL: —: C | | 1 | | |
| RET | chr10: 43600430: INDEL: —: C | | 1 | | |
| RET | chr10: 43600434: INDEL: C: — | 1 | | | |
| RET | chr10: 43606843: INDEL: G: — | | | 1 | |
| RET | chr10: 43609117: INDEL: A: — | | | | 1 |
| RET | chr10: 43615066: INDEL: —: G | 2 | 4 | | |
| RET | chr10: 43615178: INDEL: —: G | 1 | | | |
| RET | chr10: 43619121: INDEL: G: — | | | 1 | |
| RET | chr10: 43622120: INDEL: C: — | | | 1 | |
| PTEN | chr10: 89685271: INDEL: T: — | 4 | | | |
| PTEN | chr10: 89685289: INDEL: A: — | 1 | | | |
| PTEN | chr10: 89720812: INDEL: A: — | 1 | 1 | | |
| KRAS | chr12: 25368434: INDEL: T: — | | | 5 | 8 |
| KRAS | chr12: 25378575: INDEL: A: — | | 1 | | |
| KRAS | chr12: 25380194: INDEL: T: — | | | 1 | |
| AKT1 | chr14: 105242073: INDEL: CTC: — | | | 1 | 4 |
| MAP2K1 | chr15: 66736999: INDEL: —: A | 1 | | | |
| MAP2K1 | chr15: 66774094: INDEL: —: C | | 1 | | |
| MAP2K1 | chr15: 66777450: INDEL: —: T | 1 | | | |
| MAP2K1 | chr15: 66782062: INDEL: A: — | | | 10 | 10 |
| ERBB2 | chr17: 37856507: INDEL: T: — | | | 1 | |
| ERBB2 | chr17: 37856540: INDEL: C: — | | | 1 | |
| ERBB2 | chr17: 37868236: INDEL: C: — | | | 1 | |
| ERBB2 | chr17: 37868586: INDEL: —: G | 1 | 3 | | |
| ERBB2 | chr17: 37883664: INDEL: —: G | | | 1 | |
| ERBB2 | chr17: 37883664: INDEL: G: — | | | 1 | |
| ERBB2 | chr17: 37883774: INDEL: C: — | | | | 1 |
| ERBB2 | chr17: 37884218: INDEL: —: G | | | 1 | |
| SMAD4 | chr18: 48573547: INDEL: —: A | 1 | 2 | | |
| SMAD4 | chr18: 48575122: INDEL: A: — | 1 | | | |
| SMAD4 | chr18: 48575141: INDEL: —: A | 4 | 1 | | |
| SMAD4 | chr18: 48581301: INDEL: —: C | | 1 | | |
| SMAD4 | chr18: 48584778: INDEL: —: A | 1 | 1 | | |
| GNA11 | chr19: 3118934: INDEL: G: — | | | 1 | |
| GNA11 | chr19: 3119036: INDEL: G: — | | | | 1 |
| GNA11 | chr19: 3119278: INDEL: C: — | | | 1 | |
| ALK | chr2: 29416122: INDEL: T: — | | | 1 | |
| ALK | chr2: 29416157: INDEL: G: — | | | 1 | |
| ALK | chr2: 29416345: INDEL: CT: — | 1 | 1 | | |
| ALK | chr2: 29416692: INDEL: C: — | 1 | | | |
| ALK | chr2: 29443664: INDEL: C: — | | | 1 | |
| ALK | chr2: 29446231: INDEL: —: T | 2 | 1 | | |
| ALK | chr2: 29451783: INDEL: —: A | | | 7 | 2 |
| ALK | chr2: 29451815: INDEL: —: C | | 2 | | |
| ALK | chr2: 29451815: INDEL: —: C | 1 | | | |
| ALK | chr2: 29456456: INDEL: CCT: — | | | | 1 |
| ALK | chr2: 29606675: INDEL: —: G | 2 | 1 | | |
| ALK | chr2: 29917778: INDEL: —: G | 1 | | | |
| ALK | chr2: 30143052: INDEL: G: — | | | 3 | |
| ALK | chr2: 30143154: INDEL: G: — | | | | 1 |
| ALK | chr2: 30143483: INDEL: A: — | 1 | 1 | | |
| PIK3CA | chr3: 178916662: INDEL: C: — | 1 | | | |
| PIK3CA | chr3: 178916885: INDEL: —: T | 2 | 2 | | |
| PIK3CA | chr3: 178927481: INDEL: —: A | 1 | | | |
| PIK3CA | chr3: 178937764: INDEL: A: — | | | | 1 |
| PIK3CA | chr3: 178942518: INDEL: A: — | 2 | 4 | | |
| PIK3CA | chr3: 178942597: INDEL: A: — | 1 | | | |
| PIK3CA | chr3: 178948153: INDEL: —: T | 3 | 2 | | |
| CTNNB1 | chr3: 41266073: INDEL: —: A | 1 | | | |
| CTNNB1 | chr3: 41266242: INDEL: —: A | 1 | 1 | | |
| CTNNB1 | chr3: 41275197: INDEL: G: — | | | 1 | |
| CTNNB1 | chr3: 41277987: INDEL: —: A | 3 | 6 | | |
| PDGFRA | chr4: 55127345: INDEL: T: — | 1 | | | |
| PDGFRA | chr4: 55138602: INDEL: —: G | 2 | 1 | | |
| KIT | chr4: 55561719: INDEL: CCAT: — | | | | 2 |
| KIT | chr4: 55573286: INDEL: C: — | 1 | | | |
| KIT | chr4: 55589841: INDEL: T: — | 1 | 1 | | |
| KIT | chr4: 55592101: INDEL: —: T | 1 | 1 | | |
| SMO | chr7: 128829015: INDEL: —: G | | 1 | | |
| SMO | chr7: 128829015: INDEL: G: — | | 1 | | |
| SMO | chr7: 128829040: INDEL: GCT: — | | | 4 | 1 |
| SMO | chr7: 128829055: INDEL: GCT: — | | 1 | | |
| SMO | chr7: 128843237: INDEL: —: C | | | 1 | |
| SMO | chr7: 128843255: INDEL: —: C | 1 | | | |
| SMO | chr7: 128845120: INDEL: G: — | 1 | | | |
| SMO | chr7: 128850925: INDEL: —: C | 1 | | | |
| SMO | chr7: 128851514: INDEL: —: C | 1 | | | |
| SMO | chr7: 128851983: INDEL: —: C | 1 | 2 | | |
| SMO | chr7: 128851996: INDEL: —: C | 1 | 1 | | |
| SMO | chr7: 128852155: INDEL: C: — | | 1 | | |
| SMO | chr7: 128852189: INDEL: C: — | 2 | 1 | 1 | |
| BRAF | chr7: 140482927: INDEL: —: G | | | 3 | 6 |
| BRAF | chr7: 140482927: INDEL: G: — | | | 11 | 16 |
| BRAF | chr7: 140501358: INDEL: —: A | 1 | | | |
| BRAF | chr7: 140624415: INDEL: C: — | | 1 | | |
| EGFR | chr7: 55220357: INDEL: G: — | | | 1 | |
| EGFR | chr7: 55221748: INDEL: C: — | | | | 1 |
| EGFR | chr7: 55221790: INDEL: —: C | 3 | 1 | | |
| EGFR | chr7: 55249026: INDEL: —: C | 1 | 2 | | |
| EGFR | chr7: 55269429: INDEL: —: T | 1 | | | |
| EGFR | chr7: 55273266: INDEL: —: G | | 1 | | |
| JAK2 | chr9: 5050692: INDEL: —: T | 1 | 1 | | |
| JAK2 | chr9: 5050692: INDEL: —: T | | | | |
| JAK2 | chr9: 5055689: INDEL: T: — | 1 | | | |
| JAK2 | chr9: 5066679: INDEL: —: T | 3 | 4 | | |
| JAK2 | chr9: 5069022: INDEL: —: A | 1 | 1 | | |
| JAK2 | chr9: 5069060: INDEL: A: — | | | 1 | |
| JAK2 | chr9: 5069193: INDEL: C: — | | 1 | | |
| JAK2 | chr9: 5090509: INDEL: A: — | 1 | | | |
| Total false | | 72 | 61 | 65 | 57 |
| positives | | | | | |

### Assay and Sample Sensitivity and PPV Using Single Platform Analysis

Assay and sample sensitivity and PPV for SNV(s) were calculated using the 41 Paired Samples, which contained a total of 1,112 gold standard SNV(s). In addition to utilizing QUALT, MVRT, MVAF and SE thresholds/filters to optimize sensitivity and PPV for both PGM and MiSeq, we also tested 4 different variant callers [GATK (MiSeq), SVC v.2.1.12 (MiSeq), ITVC v. 3.4.51874 (PGM), and ITVC v. 3.6.63335 (PGM)]. The two with the optimal sensitivity, MiSeq SVC v.2.1.12 and PGM ITVC v. 3.6.63335, were chosen for development of thresholds and filters at the base pair level and final use in the 20-Gene validation testing (20 Gene NGS1) (FIG. 9). Default parameters from each of the manufacturers were used with the exception of those listed in the PGM Run Metrics Report. Sensitivity and PPV were first calculated for each of these four iterations of the variant caller without applying the QUALT, MVRT, MVAF and SE thresholds and filters.

Table 15 and 16 summarize the results of assay and sample sensitivity and PPV for SNV(s). The highest assay sensitivity of any platform prior to application of thresholds and filters was PGM FF at 100% for ITVC. PGM FFPE was very similar at 99% for ITVC. Application of the MVRT, QUALT, MVAF, and SE thresholds and filters resulted in a decline of assay sensitivity of 1 to 2% for PGM ITVC for both FF and FFPE. Assay sensitivity for MiSeq SVC v.2.1.12 was lower than PGM ITVC for both FF and FFPE, and application of thresholds and filters decreased sensitivity 2% in FFPE.

TABLE 15
Assay sensitivity and PPV for Paired Samples SNVs using PGM Ion Torrent Variant
Caller (ITVC) and MiSeq Reporter Somatic Variant Caller (SVC) with and without
application of QUALT, MVRT, MVAF or SE thresholds and filters.

| | | | Multi- | | | | | | | | |
|---|---|---|---|---|---|---|---|---|---|---|---|
| | | | Platform | | | | | | | | |
| | | | Filters | | | | Total | Total | Total | | |
| | Tissue | | VAF | QUAL | MVRT | MVAF | True | False | False | Assay | Assay |
| Platform | Type | MPVD | setting | Cutoff | Cutoff | Cutoff | Positive | Positive | Negative | Sensitivity | PPV |
| PGM | FF | ITVC | 0.2% | None | None | None | 1107 | 185 | 5 | 100% | 86% |
| | | v.3.6.63335 | | | | | | | | | |
| PGM | FF | ITVC | 0.2% | >99 | >=20 | >.035 | 1102 | 52 | 10 | 99% | 95% |
| | | v.3.6.63335 | | | | | | | | | |
| PGM | FFPE | ITVC | 0.2% | None | None | None | 1101 | 3002 | 11 | 99% | 58% |
| | | v.3.6.63335 | | | | | | | | | |
| PGM | FFPE | ITVC | 0.2% | >99 | >=21 | >.018 | 1078 | 106 | 34 | 97% | 91% |
| | | v.3.6.63335 | | | | | | | | | |
| MiSeq | FF | SVC | 1% | None | None | None | 1073 | 1160 | 39 | 96% | 48% |
| | | v.2.1.12 | | | | | | | | | |
| MiSeq | FF | SVC | 1% | >99 | >=5 | >.017 | 1054 | 235 | 41 | 96% | 82% |
| | | v.2.1.12 | | | | | | | | | |
| MiSeq | FFPE | SVC | 1% | None | None | None | 1044 | 16915 | 68 | 94% | 10% |
| | | v.2.1.12 | | | | | | | | | |
| MiSeq | FFPE | SVC | 1% | >99 | >=10 | >.028 | 1022 | 1619 | 90 | 92% | 39% |
| | | v.2.1.12 | | | | | | | | | |

TABLE 16
Sample sensitivity and PPV for SNVs in the Paired Samples using PGM Ion Torrent
Variant Caller (ITVC) and MiSeq Reporter Somatic Variant Caller (SVC) with and
without application of QUAL, MVRT, MVAF or SE filters.

| | | | Multi- | | | | | | | | | |
|---|---|---|---|---|---|---|---|---|---|---|---|---|
| | | Multi- | Platform | | | | Avg | Avg | | | | |
| | | Platform | Filters | | | | # | # | Mean | Range | Mean | Range |
| | Tissue | Variant | VAF | QUAL | MVRT | MVAF | False | False | Sample | Sample | Sample | Sample |
| Platform | Type | Detection | setting | Cutoff | Cutoff | Cutoff | Pos | Neg | Sensitivity | Sensitivity | PPV | PPV |
| PGM | FF | ITVC | 0.2% | None | None | None | 4.6 | 0.1 | 100% | 93-100% | 88% | 70-96% |
| | | v.3.6.63335 | | | | | | | | | | |
| PGM | FF | ITVC | 0.2% | >99 | >=20 | >.035 | 1.3 | 0.2 | 99% | 93-100% | 95% | 78-100% |
| | | v. 3.6.63335 | | | | | | | | | | |
| PGM | FFPE | ITVC | 0.2% | None | None | None | 73.2 | 0.3 | 99% | 93-100% | 58% | 2-94% |
| | | v. 3.6.63335 | | | | | | | | | | |
| PGM | FFPE | ITVC | 0.2% | >99 | >=21 | >.018 | 2.6 | 0.8 | 97% | 63-100% | 92% | 40-100% |
| | | v. 3.6.63335 | | | | | | | | | | |
| MiSeq | FF | SVC | 1% | None | None | None | 28.3 | 1.0 | 97% | 79-100% | 49% | 31-66% |
| | | v.2.1.12 | | | | | | | | | | |
| MiSeq | FF | SVC | 1% | >99 | >=5 | >.017 | 5.7 | 1.0 | 95% | 66-100% | 82% | 66-95% |
| | | v.2.1.12 | | | | | | | | | | |
| MiSeq | FFPE | SVC | 1% | None | None | None | 420.9 | 1.7 | 94% | 43-100% | 10% | 2-37% |
| | | v.2.1.12 | | | | | | | | | | |
| MiSeq | FFPE | SVC | 1% | >99 | >=10 | >.028 | 39.5 | 2.2 | 92% | 39-100% | 62% | 6-100% |
| | | v.2.1.12 | | | | | | | | | | |

Assay PPV was in contrast to sensitivity much lower for all platforms ranging from a low of 10% for MiSeq FFPE to a high of 86% for PGM FF prior to application of thresholds and filters. After application of thresholds and filters the greatest improvement in PPV was noted for PGM FFPE, changing from 58% to 91%. The majority of false positive calls on the PGM FFPE platform are from low quality reads, explaining the marked improvement in assay PPV following the application of MVRT, QUALT, MVAF, and SE thresholds and filters, with only a small corresponding 2% decrease in assay sensitivity. A modest gain in PPV was also shown for PGM FF from 86% to 95%. Similarly, application of thresholds and filters significantly improved assay PPV for both MiSeq FF and FFPE from 48% to 82%, and 10% to 39% respectively.

Mean sample sensitivity (Table 16) followed the same trends as assay sensitivity. For PGM FF and FFPE, the minimum sensitivity for any given sample was 93% prior to application of thresholds and filters. Application of MVRT, QUALT, MVAF and SE thresholds and filters had no impact for PGM FF sample sensitivity, however, for PGM FFPE, the minimum sample sensitivity decreased 30% with application of MVRT, QUALT, MVAF, and SE thresholds and filters. Sample sensitivity for MiSeq showed a much greater range of values than did the PGM. For MiSeq FF and FFPE, minimum sample sensitivity was 79% and 43% respectively, with no application of thresholds or filters. Application of the MVRT, QUALT, MVAF, and SE thresholds and filters for MiSeq resulted in slight decreases to sample sensitivity from 43% to 39% for FFPE samples and from 79% to 66% for FF samples.

Mean sample PPV was highest for PGM FF at 88% and lowest for MiSeq FFPE prior to application of the filters (Table 16). The range of sample level PPV varied widely by platform and sample type prior to application of thresholds and filters, with the greatest range being that for PGM FFPE (2%-94%). Application of MVRT, QUALT, MVAF, and SE thresholds and filters increased the minimum sample PPV across the board, however, a wide, and in the case of MiSeq FFPE, even wider (from 2%-37% to 6%-100%) range of values was observed following application of filters and thresholds, indicating suboptimal performance of SVC v.2.1.12 in this regard.

Unlike FF samples, there were FFPE samples that did not meet our goal of having at least 250× mean depth or coverage. To evaluate the impact, we investigated the association of mean depth or coverage with sample sensitivity and PPV for FFPE samples. For both platforms, we found that the subset of samples with mean depth or coverage below 250× also had lower PPV and sensitivity than those with at least 250× mean depth or coverage, with the greatest difference, 35 percentage points, shown for PGM PPV (28% vs. 63%) prior to application of thresholds and filters (Table 17).

table 17
Sample sensitivity and PPV above and below 250× depth or coverage for the FFPE
Paired Samples GS using PGM Ion Torrent Variant Caller (ITVC) and MiSeq Reporter
Somatic Variant Caller (SVC) with and without application of QUALT, MVRT, MVAF
or SE filters.

| | | | Post | | | | | | |
|---|---|---|---|---|---|---|---|---|---|
| | | | Multi- | | | Mean | Mean | Mean | Mean |
| | | Multi- | Platform | | | sample | sample | sample | sample |
| | | Platform | Variant | Mean | Mean | Sensitivity | PPV | Sensitivity | PPV |
| | Tissue | Variant | Detection | Sample | Sample | >250× | >250× | <250× | <250× |
| Platform | Type | Detection | Filters | Sensitivity | PPV | depth | depth | depth | depth |
| PGM | FFPE | ITVC | none | 99% | 58% | 99% | 63% | 97% | 28% |
| | | v.3.6.63335 | | | | | | | |
| PGM | FFPE | ITVC | Yes | 98% | 89% | 99% | 93% | 86% | 87% |
| | | v.3.6.63335 | | | | | | | |
| MiSeq | FFPE | SVC | none | 94% | 10% | 94% | 10% | 75% | 3% |
| | | v.2.1.12 | | | | | | | |
| MiSeq | FFPE | SVC | Yes | 92% | 53% | 92% | 63% | 66% | 24% |
| | | v.2.1.12 | | | | | | | |

The application of thresholds and filters increased PPV across the board, but it had the greatest effect on PPV for MiSeq FFPE samples with <250× mean depth or coverage, showing an increase from 24% to 63%. Again, in single platform analysis, huge increases in mean sample PPV through the application of thresholds and filters came at a cost to mean sample sensitivity, with the greatest decrease of 11% shown for PGM FFPE samples (from 86% to 97%).

In an additional effort to improve single platform PPV, the minimum requirements for number of variant reads was investigated that allow for 95% confidence that any variant call is a true positive and not a false positive. The intent of this process was to develop an equivalent MVAR for PPV, or analytical PPV (APPV), as we had previously developed for sensitivity. To do so we calculated cumulative PPV using the Pooled Sample for multiple replicates across multiple runs for the 16 replicates (libraries) of the Pooled Sample for the MiSeq FF, 12 for MiSeq FFPE, and 20 each for PGM FF and FFPE. Cumulative PPV for a given sequencing platform and specimen type was calculated for an ascending ranking of all variants by number of variant reads and then identifying that number of variant reads where PPV was below 95%. For PGM FF and FFPE, 23 and 18 minimum variant reads, respectively, were required to have 95% confidence that a variant was not a false positive (FIGS. 10A-D). FIGS. 10A-D are graphical representations showing MVAF for each sequencing platform and tissue fixation type. The lowest VAF for analytical sensitivity was achieved with MiSeq FF (1.7% VAF). The value for PGM FFPE at 1.8% VAF was similar and with only minor differences to PGM FF (2.9% VAF) and MiSeq FFPE (3.6% VAF).

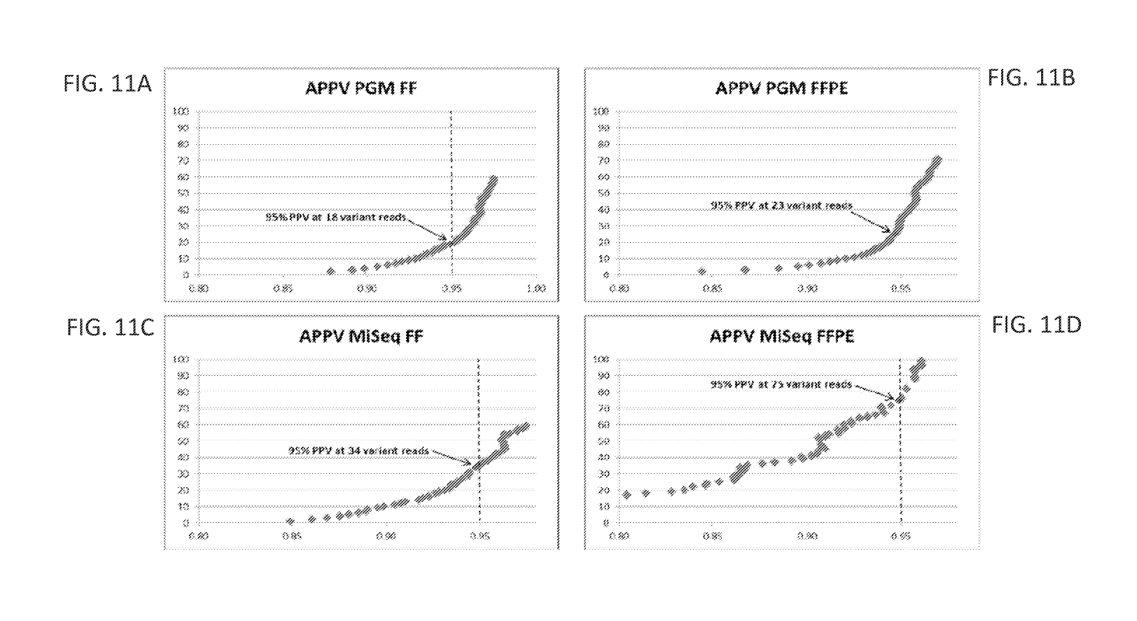

These values are relatively close to the values of 21 and 20 for MVAR for sensitivity for PGM FF and FFPE, respectively. For MiSeq FF and FFPE, the corresponding values were more than 10× these values and not close to any practical application. By capping the total BAM reads to less than 1,000 for MiSeq FF and FFPE the analysis showed that 34 and 75 variant reads were required for 95% confidence that a variant was not a false positive, versus the values of 5 and 10 for MVAR for sensitivity, respectively. The much higher numbers for analytical PPV for MiSeq FF and FFPE, and the need to cap total BAM reads to less than 1,000 is shown by plotting PPV against total BAM reads for the same datasets (FIGS. 11A-D). FIGS. 11A-D are graphical representations of analytical positive predictive value (PPV) for single nucleotide variants in the 20-gene validation testing. For both MiSeq FF and FFPE, as opposed to PGM FF and FFPE, PPV declines at high levels of coverage and similar to PGM FF and FFPE peaks at levels of 1-2,000× coverage. Application of an equivalent MVAR for PPV as for sensitivity has minimal impact on assay sensitivity for PGM, but markedly decreases sensitivity for MiSeq due to the much higher cut-off values and is not a practical solution.

The analysis of assay and sample sensitivity and PPV for indels in the Paired Samples was simplified by the limited number of indels, and the ability to greatly exclude false positives at no impact to true positives by application of the QUALT. As discussed herein, the average number of false positive indels per sample was relatively close for all sequencing platforms and tissue fixation type at 3.6 for Miseq FF, 5.4 for MiSeq FFPE, 3.0 for PGM FF, and 3.6 for PGM FFPE per run, but these numbers were greatly reduced to 0.17, 0.37, 0.07, and 0.07, respectively, after application of the QUALT. Similar to the results for the Pooled Sample, assay sensitivity for detection of indels in the Paired Samples for MiSeq FF and MiSeq FFPE was 100% (Table 18). Assay sensitivity for PGM FF and MiSeq FFPE at 60% each was improved over the results for the Pooled Sample likely due to a higher VAF of the known gold standard indels. Assay PPV for all sequencing platforms and tissue fixation types after applying thresholds and filters was still less than optimal ranging from a high of 50% for PGM FF and FFPE to a low of 25% for MiSeq FFPE.

TABLE 18
Assay sensitivity and PPV for indels in the Paired Samples using PGM Ion Torrent
Variant Caller (ITVC) and MiSeq Reporter Somatic Variant Caller (SVC) with and
without application of QUALT, MVRT, MVAF or SE filters.

| | | | Multi- | | | | | | | | |
|---|---|---|---|---|---|---|---|---|---|---|---|
| | | Multi- | Platform | | | | | | | | |
| | | Platform | Filters | | | | Total | Total | Total | | |
| | Tissue | Variant | VAF | QUAL | MVRT | MVAF | True | False | False | Assay | Assay |
| Platform | Type | Detection | setting | Cutoff | Cutoff | Cutoff | Pos | Pos | Neg | Sensitivity | PPV |
| PGM | FFPE | ITVC | 0.2% | None | None | None | 3 | 3 | 2 | 60% | 2% |
| | | v.3.6.63335 | | | | | | | | | |
| PGM | FFPE | ITVC v. | 0.2% | >99 | None | None | 3 | 3 | 2 | 60% | 50% |
| | | 3.6. 63335 | | | | | | | | | |
| PGM | FF | ITVC v. | 0.2% | None | None | None | 3 | 3 | 2 | 60% | 50% |
| | | 3.6. 63335 | | | | | | | | | |
| PGM | FF | ITVC v. | 0.2% | >99 | None | None | 3 | 3 | 2 | 60% | 50% |
| | | 3.6. 63335 | | | | | | | | | |
| MiSeq | FFPE | SVC | 1% | None | None | None | 5 | 15 | 0 | 100% | 2% |
| | | v.2.1.12 | | | | | | | | | |
| MiSeq | FFPE | SVC | 1% | >99 | None | 0.036 | 5 | 15 | 0 | 100% | 25% |
| | | v.2.1.12 | | | | | | | | | |
| MiSeq | FF | SVC | 1% | None | None | None | 5 | 7 | 0 | 100% | 4% |
| | | v.2.1.12 | | | | | | | | | |
| MiSeq | FF | SVC | 1% | >99 | None | 0.019 | 5 | 7 | 0 | 100% | 42% |
| | | v.2.1.12 | | | | | | | | | |

While all samples in the Paired Samples with a gold standard indel were also included in the Pooled Sample, a higher VAF for these variants in the former is not the complete explanation for an improvement in their detection. In Table 19, all gold standard indels in the Paired Samples were sorted by sequencing platform and tissue fixation type and then ranked by ascending percent variant reads in the BAM file. From this detailed information, it was apparent that the two indels missed by PGM are common to both FF and FFPE fixation tissue types, but in all instances there was adequate coverage and number of variant reads in the BAM file to potentially make the correct call. A detailed review of the BAM pileups showed that the majority of bases at all these locations was of Q20 or greater. While various parameters in ITVC can be set at different values in the Ion Torrent Variant Suite, raw sequencing data was not readily exportable to test with other variant callers. Given the proprietary nature of the PGM and Ion Torrent Variant Suite, as a single sequencing platform there is limited detection of indels with the current system. In the clinical scenario, all of these discordant indels in the PGM would be detected upon manual review of the BAM pileup using a computer-based inspection tool that enables the manual review of the BAM pileup information.

TABLE 19
Results for detection of gold standard indels in the Paired Samples sorted by sequencing
platform and tissue fixation type and ranked by ascending percent variant reads in the
BAM file.

| | | | | | | | | |
|---|---|---|---|---|---|---|---|---|
| | | | | | | | | Percent |
| | | | | | Total | Variant | *Variant | variant |
| | | | | *VAF | BAM | reads | reads | reads |
| TCGAID | Gene | Variant | Detected | VCF | Reads | BAM | VCF | BAM |

MiSeq FF

| | | | | | | | | |
|---|---|---|---|---|---|---|---|---|
| TCGA- | SMAD4 | chr18:48584513: | Yes | 0.11 | 2231 | 338 | 328 | 0.15 |
| G4- | | Indel:TG:T | | | | | | |
| 6309 | | | | | | | | |
| TCGA- | PTEN | chr10:89717769: | Yes | 0.33 | 4003 | 1328 | 1297 | 0.33 |
| G4- | | Indel:TA:T | | | | | | |
| 6309 | | | | | | | | |
| TCGA- | PTEN | chr10:89692825: | Yes | 0.35 | 283 | 98 | 95 | 0.35 |
| G4- | | Indel:CT:C | | | | | | |
| 6309 | | | | | | | | |
| TCGA- | CTNNB1 | chr3:41266133: | Yes | 0.26 | 2832 | 997 | 721 | 0.35 |
| G4- | | Indel:CCTT:C | | | | | | |
| 6586 | | | | | | | | |
| TCGA- | PTEN | chr10:89717727: | Yes | 0.48 | 4778 | 2316 | 2245 | 0.48 |
| E2- | | Indel:GTGAT | | | | | | |
| A14Z | | ATCAAA:- | | | | | | |

MiSeq FFPE

| | | | | | | | | |
|---|---|---|---|---|---|---|---|---|
| TCGA- | SMAD4 | chr18:48584513: | Yes | 0.16 | 2361 | 488 | 473 | 0.21 |
| G4- | | Indel:TG:T | | | | | | |
| 6309 | | | | | | | | |
| TCGA- | PTEN | chr10:8971776: | Yes | 0.25 | 3633 | 905 | 906 | 0.25 |
| G4- | | Indel:TA:T | | | | | | |
| 6309 | | | | | | | | |
| TCGA- | PTEN | chr10:89692825: | Yes | 0.30 | 307 | 93 | 88 | 0.30 |
| G4- | | Indel:CT:C | | | | | | |
| 6309 | | | | | | | | |
| TCGA- | CTNNB1 | chr3:41266133: | Yes | 0.20 | 398 | 145 | 75 | 0.36 |
| G4- | | Indel:CCTT:C | | | | | | |
| 6586 | | | | | | | | |
| TCGA- | PTEN | chr10:89717727: | Yes | 0.47 | 1061 | 499 | 481 | 0.47 |
| E2- | | Indel:GTGAT | | | | | | |
| Al4Z | | ATCAAA:- | | | | | | |

PGM FF

| | | | | | | | | |
|---|---|---|---|---|---|---|---|---|
| TCGA- | CTNNB1 | chr3:41266133: | Yes | 0.23 | 567 | 131 | 131 | 0.23 |
| G4- | | Indel:CCTT:C | | | | | | |
| 6586 | | | | | | | | |
| TCGA- | PTEN | chr10:89692825: | Yes | 0.37 | 1089 | 306 | 281 | 0.28 |
| G4- | | Indel:CT:C | | | | | | |
| 6309 | | | | | | | | |
| TCGA- | PTEN | chr10:89717769: | No | 0.00 | 945 | 327 | 0 | 0.35 |
| G4- | | Indel:TA:T | | | | | | |
| 6309 | | | | | | | | |
| TCGA- | SMAD4 | chr18:48584513: | No | 0.00 | 1201 | 571 | 0 | 0.48 |
| G4- | | Indel:TG:T | | | | | | |
| 6309 | | | | | | | | |
| TCGA- | PTEN | chr10:89717727: | Yes | 0.50 | 806 | 396 | 393 | 0.49 |
| E2- | | Indel:GTGAT | | | | | | |
| A14Z | | ATCAAA:- | | | | | | |

PGM FFPE

| | | | | | | | | |
|---|---|---|---|---|---|---|---|---|
| TCGA- | PTEN | chr10:89692825: | Yes | 0.33 | 759 | 189 | 186 | 0.25 |
| G4- | | Indel:CT:C | | | | | | |
| 6309 | | | | | | | | |
| TCGA- | CTNNB1 | chr3:41266133: | Yes | 0.26 | 290 | 74 | 74 | 0.26 |
| G4- | | Indel:CCTT:C | | | | | | |
| 6586 | | | | | | | | |
| TCGA- | PTEN | chr10:89717769: | No | 0.00 | 610 | 175 | 0 | 0.29 |
| G4- | | Indel:TA:T | | | | | | |
| 6309 | | | | | | | | |
| TCGA- | SMAD4 | chr18:48584513: | No | 0.00 | 868 | 433 | 0 | 0.50 |
| G4- | | Indel:TG:T | | | | | | |
| 6309 | | | | | | | | |
| TCGA- | PTEN | chr10:89717727: | Yes | 0.56 | 695 | 380 | 378 | 0.55 |
| E2- | | Indel:GTGAT | | | | | | |
| A14Z | | ATCAAA:- | | | | | | |

The analysis of sensitivity and PPV for indels was further evaluated by sequencing the 15 clinical lung cancer EGFR Samples, consisting of seven (7) samples harboring five (5) unique exon 19 EGFR indels and eight (8) samples with no EGFR indel detected. Similar to the Paired Samples results, high assay sensitivity (86%) was achieved for the MiSeq FFPE with 6 exon 19 indels detected in the 7 EGFR indel positive samples (Table 20). One sample, M-11-02006, for which the exon 19 indel was not detected, manual review of the BAM file for both MiSeq and PGM showed no evidence of variant calls (Table 21). This result was equivalent to an update to the gold standard and a final MiSeq FFPE assay sensitivity of 100%. The lack of an EGFR indel in this sample is explained by the fact that this was the only sample in the EGFR Samples where the clinical sample was a biopsy and the validation sample was a different resection specimen, supporting the concept of tumor heterogeneity. There were no MiSeq FF results for the EGFR Samples as the only samples available for testing were FFPE blocks.

TABLE 20
Summary of assay sensitivity and PPV for indels in EGFR using the EGFR Samples
with SVC v.2.1.12 (MiSeq) and ITVC v3.6.63335 with QUAL and MVAF thresholds
and exclusion of systematic errors (SE).

| | | | Multi- | | | | | | | | | |
|---|---|---|---|---|---|---|---|---|---|---|---|---|
| | | Multi- | Platform | | | | | | | | | |
| | | Platform | Filter | | | | Total | Total | Total | | | |
| | Tissue | Variant | VAF | QUAL | MVRT | MVAF | True | False | False | Assay | Assay | Supp. |
| Platform | Type | Detection | setting | Cutoff | Cutoff | Cutoff | Positive | Positive | Negative | Sensitivity | PPV | Data |
| PGM | FFPE | ITVC | 0.2% | >99 | None | None | 0 | 0 | 6 | 0% | NA | S80 |
| | | v.3.6.63335 | | | | | | | | | | |
| MiSeq | FFPE | SVC | 1% | >99 | None | 0.037 | 6 | 3 | 1 | 86% | 65% | S79 |
| | | v.2.1.12 | | | | | | | | | | |

The analysis of sensitivity and PPV for PGM FFPE indels was suboptimal as none of the putative variants were called. In the same manner as the MiSeq FFPE, inspection of the PGM BAM files clearly show the variants are present in the 6 EGFR Samples containing the exon 19 indel, but the ITVC failed to make the correct variant call (Table 21). Closer inspection of the amplicon sequences for EGFR exon 19 show that the start/stop locations for the two amplicons that target exon 19 reside in the 18 bp indel hotpot region. This effectively results in most reads unable to align correctly and nearly 100% strand bias due to map trimming. Fortunately, this scenario is accounted for in the clinical setting for 20 Gene NGS1 during the manual review step described above. Nonetheless for this validation, the assay sensitivity of indel detection for PGM FFPE for the EGFR Samples was 0% (Table 21).

TABLE 21
EGFR Samples indel detection ranked by ascending percent variant reads in the BAM
file.

| | | | | | | | |
|---|---|---|---|---|---|---|---|
| | | Gold | | | | | Percent |
| | | standard | | Total | Variant | *Variant | variant |
| | | classi- | *VAF | BAM | reads | reads | reads |
| TCGAID | Variant | fication | VCF | Reads | BAM | VCF | BAM |

MiSeq FFPE

| | | | | | | | |
|---|---|---|---|---|---|---|---|
| M-12- | 7:55242467- | True | 4.81% | 1546 | 74 | 73 | 4.79% |
| 00517 | 55242484:AATTAAGAGAAGCAAC | Positive | | | | | |
| | A:- | | | | | | |
| M-11- | 7:55242470- | True | 22.83% | 852 | 196 | 189 | 23.00% |
| 01880 | 55242488:TAAGAGAAGCAACATC | Positive | | | | | |
| | TC:- | | | | | | |
| M-12- | 7:55242465- | True | 41.11% | 3388 | 1375 | 1401 | 41.35% |
| 03077 | 55242480:GGAATTAAGAGAAGC:- | Positive | | | | | |
| M-12- | 7:55242467- | True | 45.13% | 510 | 238 | 227 | 46.67% |
| 02970 | 55242484:AATTAAGAGAAGCAAC | Positive | | | | | |
| | A:- | | | | | | |
| M-12- | 7:55242466- | True | 66.69% | 4315 | 2939 | 2835 | 68.11% |
| 02954 | 55242481:GAATTAAGAGAAGCA:- | Positive | | | | | |
| M-11- | 7:55242470- | True | 69.95% | 6476 | 4543 | 4451 | 70.15% |
| 01054 | 55242488:TAAGAGAAGCAACATC | Positive | | | | | |
| | TC:- | | | | | | |
| M-11- | 7:55242466- | False | | 1053 | 0 | | 0.00% |
| 02006 | 55242481 :GAATTAAGAGAAGCA:- | Negative | | | | | |
| M-12- | 7:55242467- | False | | 273 | 17 | | 6.23% |
| 00517 | 55242484 :AATTAAGAGAAGCAAC | Negative | | | | | |
| | A:- | | | | | | |
| M-11- | 7:55242470- | False | | 549 | 185 | | 33.70% |
| 01880 | 55242488:TAAGAGAAGCAACATC | Negative | | | | | |
| | TC:- | | | | | | |
| M-12- | 7:55242465- | False | | 815 | 117 | | 14.36% |
| 03077 | 55242480:GGAATTAAGAGAAGC:- | Negative | | | | | |
| M-12- | 7:55242467- | False | | 662 | 151 | | 22.81% |
| 02970 | 55242484:AATTAAGAGAAGCAAC | Negative | | | | | |
| | A:- | | | | | | |
| M-12- | 7:55242466- | False | | 780 | 213 | | 27.31% |
| 02954 | 55242481:GAATTAAGAGAAGCA:- | Negative | | | | | |
| M-11- | 7:55242470- | False | | 1833 | 1127 | | 61.48% |
| 01054 | 55242488:TAAGAGAAGCAACATC | Negative | | | | | |
| | TC:- | | | | | | |
| M-11- | 7:55242466- | False | | 1321 | 0 | | 0.00% |
| 02006 | 55242481:GAATTAAGAGAAGCA:- | Negative | | | | | |

*Extracted from VCF; Extracted from BAM file.

False positive indels in the EGFR Samples were only identified in the MiSeq FFPE and limited to four unique single base pair deletions outside the actionable region of 7:55,242,470-55,242,481 where more than 90% of all EGFR mutations and the vast majority of indels occur (Table 22). Assay PPV for MiSeq FFPE based upon these results was 65%, and no similar result could be calculated for PGM FFPE.

TABLE 22
EGFR false positive indels ranked by ascending VAF.

| | | | | Gold | | REF | Variant |
|---|---|---|---|---|---|---|---|
| | | | Sample | Standard | VAF | Reads | Reads |
| SampleID | Variant | Platform | Type | Classification | VCF | VCF | VCF |
| M-11-01508 | 7:55269031- | MiSeq | FFPE | False Positive | 2.24% | 803 | 17 |
| | 55269032:C: | | | | | | |
| M-12-02970 | 7:55221748- | MiSeq | FFPE | False Positive | 3.74% | 214 | 8 |
| | 55221749:C: | | | | | | |
| M-12-00517 | 7:55242485- | MiSeq | FFPE | False Positive | 4.78% | 1547 | 73 |
| | 55242486:C: | | | | | | |
| M-12-02970 | 7:55242485- | MiSeq | FFPE | False Positive | 46.56% | 498 | 230 |
| | 55242486:C: | | | | | | |

### Summary of Single Platform Analysis

As a summary of assay and sample sensitivity and PPV for SNV(s) using a single sequencing platform strategy, the first major conclusion from this validation was that sensitivity is less of a problem than PPV. In regard to the latter, application of post-variant calling filters for SNV(s) for the MiSeq resulted in less improvement of assay PPV and a slightly greater decrease in assay sensitivity than for PGM. This is related to the fact that false positive calls for the MiSeq, while more frequent among variants with poor quality reads, are relatively more frequent at high levels of coverage considered as high quality reads than for the PGM. The thresholds and filters of MVRT, QUALT, SE and MVAF greatly improve the results of single platform analysis and are used advantageously in the multi-platform detection methods and systems to diminish the number of variants requiring manual review.

The second major conclusion from this validation was that PGM data, derived from ITVC, was an inadequate method for detection of indels as a single sequencing platform. In contrast, MiSeq for both FF and FFPE performed exceptionally well. Due to the limited number of indels in this validation study, we were unable to define a MVRT for this variant type in either platform, however thresholds and filters for QUALT and SE were readily applied to both. A MVAF could only be defined for MiSeq, but not for PGM due to the lack of detection of indels by the latter.

The third major conclusion was neither of the NGS platforms, individually for FFPE, could achieve our targeted goal of 95% sensitivity and 95% PPV. This resulted in the development of the disclosed methods and systems.

### Variant Calling Methods and Systems

The 20 Gene NGS1 analysis techniques are one embodiment of the variant calling methods and systems described herein. The methods and systems utilize an individual sample sequenced for the same target regions on both the MiSeq and PGM to produce one final result set with the intent of optimizing both sensitivity and PPV. Accounting for predetermined SEs, the multi-platform detection methods and systems was designed to address the inherent nature of random false positive calls for both PGM and MiSeq. The methods and systems include a predetermined list of actionable variants in the clinical setting or gold standard variants in the validation setting. The disclosure utilizes pre-multi-platform detection methods and systems data from the VCF and BAM files from both sequencing platforms to classify calls based upon MVRT, QUALT, MVAF and SE thresholds and filters. The techniques disclosed herein correctly increase the variant calling results of not detected (ND) and failed testing (FT).

### Analysis in the Validation Setting

As discussed herein, in the validation of the 20 Gene NGS1, the methods and systems of classification of variants followed a predetermined set of, with those for non-gold standard variants being simpler than those for gold standard variants (Table 23).

TABLE 23
Analysis for calculating sensitivity and PPV.

| | | | Passes All | | MiSeq | PGM | Decision |
|---|---|---|---|---|---|---|---|
| | MiSeq | PGM | Thresholds of the | | Classification | Classification | of the |
| | Pre-Multi- | Pre-Multi- | Multi-Platform | | of the Multi- | of the Multi- | Multi- |
| | Platform | Platform | Detection | | Platform | Platform | Platform |
| | Detection | Detection | MiSeq | PGM | Detection | Detection | Detection |
| Gold | TP | TP | Yes | Yes | TP | TP | TP |
| standard | TP | TP | Yes | No | TP | FN | TP |
| variants | TP | TP | No | Yes | FN | TP | TP |
| | TP | TP | No | No | FN | FN | FN |
| | TP | FN | Yes | NA | TP | FN | TP |
| | TP | FN | No | NA | FN | FN | FN |
| | FN | TP | NA | Yes | FN | TP | TP |
| | FN | TP | NA | No | FN | FN | FN |
| | FN | FN | NA | NA | FN | FN | FN |
| Non-gold | FP | FP | Yes | Yes | FP | FP | FP |

| | |
|---|---|
| standard | All other non-gold standard variants NR. |

TP = True Positive,
FP = False positive,
FN = False negative,
NA = Not Applicable
NR = Not Reported

A non-gold standard variant requires detection by both sequencing platforms in a given sample in order to enter the multi-platform (MPVD) methods and systems for classification. To be classified as a confirmed false positive (FP) by the MP detection, a non-gold standard variant was required to pass all MP thresholds and filters (MVRT, QUALT, SE and MVAR); otherwise, the variant was not reported (NR). For gold standard variants, the MPVD classification techniques are divided into concordant false negative (FN) variants versus all other possibilities. For concordant false negative variants, the final MPVD classification was always false negative. For all other possible combinations of gold standard variant calls, the MPVD classification of the variant was dependent upon the call passing all platform and sample-type specific MPVD thresholds and filters (MVRT, QUALT, SE, and MVAR). Any true positive (TP) variant that fails any one of these thresholds or filters for a specific sample type and platform is converted to a false negative call prior to entering the MPVD. Pre-MPVD concordant true positive variant calls for which both calls fail one or more of the MPVD thresholds and filters results in a false negative MPVD decision. Pre-MPVD concordant true positive calls for which only one call fails one or more of the thresholds and filters results in a true positive MPVD decision. The MPVD decision for a pre-MPVD discordant true positive call where one platform reports a true positive and the other a false negative, and for which the true positive fails any one of the thresholds and filters results in a false negative MPVD decision. Conversely for this latter situation if the true positive passes all of the thresholds and filters the MPVD decision is true positive.

### Comparison of Variant Detection Methods in the Validation Versus the Clinical Setting

In the clinical setting, gold standard and non-gold standard variants are replaced by actionable and non-actionable variants, respectively. Similar to the treatment of gold standard variants in the validation setting, all actionable variants in the VCF file are accepted for further analysis by the MPVD in the clinical setting whether concordant or discordant across the two sequencing platforms. All non-actionable variants in the VCF file must be concordant across both platforms for further analysis by the MPVD, similar to false positives in the validation setting. An exception to this in the validation setting is that concordant false negatives that are not reported in the VCF file are allowed to enter the MPVD to avoid artificially increasing sensitivity. In the validation setting, the sum of true positive and false negative calls as classified by the MPVD is always equal to the sum of all gold standard variant calls. The MPVD can convert concordant true positive calls to a false negative MPVD decision; however, a concordant false negative always remains a false negative. In the clinical setting the equivalent of a false negative is not detected (ND), or an actionable variant not reported in the VCF. Therefore, the sum of MUT and ND calls after MPVD classification for actionable variants is equal to the sum of all actionable calls.

Additional comparisons of variant detection methods in the validation versus the clinical setting are focused on preliminary classification calls passing the criteria of MVRT, QUALT, SE, and MVAF thresholds and filters. An actionable variant in the clinical setting with concordant MUT calls where at least one of the calls meets all the MVRT, QUALT, SE and MVAF criteria, is sufficient for a MUT MPVD decision. This is the equivalent scenario for gold standard concordant true positive calls in the validation setting, which results in a true positive MPVD decision. An actionable variant in the clinical setting with concordant MUT calls where both calls do not meet MVRT, QUALT, SE and MVAF criteria, results in a FT or ND MPVD decision. This is the equivalent scenario for gold standard concordant true positive calls in the validation setting, which results in a false negative MPVD decision. Similarly, a discordant actionable variant (MUT/FT or MUT/ND) for which the MUT call passes all the MVRT, QUALT, SE and MVAF criteria, is sufficient for a MPVD decision of MUT. The equivalent scenario for gold standard concordant true positive calls in the validation setting results in a true positive MPVD decision. Compared to the validation setting, a discordant MUT variant in the clinical setting requires a third technology confirmation such as pyrosequencing or Sanger sequencing. A discordant actionable variant (MUT/FT or MUT/ND) for which the MUT call does not pass all the MVRT, QUALT, SE and MVAF results in a FT or ND validation call. The equivalent scenario for gold standard concordant true positive calls in the validation setting results in a false negative MPVD decision.

Sensitivity and PPV for SNV(s) was calculated in the MPVD utilizing the same set of 41 samples as used for single platform analysis (Paired Samples), which contained a total of 1,112 gold standard SNV(s) (true positives and false negatives) and a variable number of false positives dependent upon the platform and fixation tissue type. The MPVD utilizes both a MiSeq and PGM run to produce one final result set for FF and FFPE specimens that is confined to the target regions of 20 Gene NGS1. Due to the inherent nature of false positive calls being random for both PGM and MiSeq, with the exception of predetermined SEs, the techniques employed by the MPVD are designed to take advantage of this fact and maximize PPV with minimal impact on sensitivity. Within the MPVD, variant calls are filtered using the QUAL, MVRT, SE, and MVAR filters in the same fashion as for single platform analysis, resulting in reclassification followed by a single MPVD decision for the paired calls using a predefined set of engine rules (Table 24).

In the clinical setting the fundamental rules of the MPVD are that all actionable variants in the VCF are accepted for further analysis whether concordant or discordant for the two sequencing platforms. All non-actionable variants in the VCF require a concordant status for further analysis in the MPVD, otherwise they are excluded. In the validation setting actionable and non-actionable variants are replaced by gold standard and non-gold standard variants, respectively. In this fashion concordant false negatives, which are not reported in the VCF, are allowed to enter the MPVD to avoid artificially increasing sensitivity. For actionable variants in the clinical setting (mutation/MUT, not detected/ND, failed testing/FT), or gold standard variants in the validation setting (true positive/TP, false negative/FN, false positive/FP), a concordant TP or MUT call for which at least one call meets all the criteria of MVRT, QUALT, SE and MVAF is sufficient for a TP MPVD decision. In a similar fashion, a discordant TP/FN gold standard variant, equivalent to a MUT/FT or MUT/ND actionable variant, for which the TP or MUT call passes all the criteria of MVRT, QUALT, SE and MVAF is sufficient for a TP call or MUT in the MPVD. A discordant TP/FN gold standard variant, or MUT/FT or MUT/ND actionable variant in the clinical setting, for which the TP call does not pass MVRT, QUALT, SE and MVAF results in a FN validation call, or equivalent ND or FT clinical call, in the MPVD. In the validation setting concordant FN calls result in a FN call in the MPVD regardless of MVRT, QUALT, SE and MVAF filters, which are not applicable. In the clinical setting FN(s) are equivalent to actionable variants not reported in the VCF and if pass QUAL and MVRT are not otherwise reported, but included in the validation to reflect the actual assay sensitivity. Concordant FP calls in the validation setting result in a FP call in the MPVD when both FP calls pass all the criteria of MVRT, QUALT, SE and MVAF, otherwise they are not reported. Discordant FP calls are not reported in the MPVD in the validation setting. In the clinical setting the MPVD decision for non-actionable variants are managed in a fashion similar to FP calls in the validation setting.

### Assay and Sample Sensitivity and PPV Using the Multi-Platform Detection Methods and Systems

For FF MiSeq SVC v. 2.1.12 and PGM ITVC v3.6.63335 there were 2,426 MPVD decisions for SNV(s), of which slightly more than one-half (1,314; 54%) were a false positive call in either one or both platforms (Table 24). The majority of these false positive calls (1,123; 85%) were excluded from further analysis by failing one or more of the MVRT, QUALT, SE and MVAF thresholds or filters. In the MPVD output the remaining 191 false positive calls were reduced to 29, or 2% of the total, as discordant and non-actionable by classification resulting in an assay PPV of 97.5%. For the 1,111 true positives by both platforms the vast majority (1,110; 99.9%) passed all the criteria of MVRT, QUALT, SE and MVAF filters. For discordant true positives (total=42) the true positive call passed all the criteria of MVRT, QUALT, SE and MVAF filters for all calls (100%). There was only one concordant false negative, for which no filters are applicable and resulting in a MPVD false negative call. For the 42 discordant TP/FN or FN/TP calls, only 12 (18%) failed any of the criteria of MVRT, QUALT, SE and MVAF, resulting in a low number of MPVD false negative calls. The MPVD assay sensitivity and PPV for FF MiSeq SVC v. 2.1.12 and PGM ITVC v3.6.63335 of 99.8% and 97.5%, respectively, is better than the comparable single platform values for FF MiSeq SVC v. 2.1.12 of 99% and 95% and PGM ITVC V3.6.63335 of 96% and 82%, respectively.

TABLE 24
MPVD results for FF specimens for SNV(s) in the Paired Samples.

| | | Pre-Multi- | | Within | | | | | | | |
|---|---|---|---|---|---|---|---|---|---|---|---|
| | | Platform | | Multi-Platform | | Multi-Platform Detection Decision | | | | | Total |
| | | Detection | | Detection | | True | False | False | Not | Failed | Detection |
| | | MiSeq | PGM | MiSeq | PGM | Positive | Positive | Negative | Reported | Testing | Decisions |
| Concordant | gold | TP | TP | TP | TP | 1046 | | | | | 1046 |
| | standard | TP | TP | TP | FN | 4 | | | | | 4 |
| | | TP | TP | FN | TP | 18 | | | | | 18 |
| | | TP | TP | FN | FN | | | 1 | | 1 | 1 |
| | | FN | FN | FN | FN | | | 1 | | | 1 |
| | Non-gold | FP | FP | ND | ND | | | | 1 | 1 | 1 |
| | | FP | FP | ND | FP | | | | 1 | 1 | 1 |
| | | FP | FP | FP | ND | | | | | 0 | 0 |
| | | FP | FP | FP | FP | | 29 | | | | 29 |
| | | | | | Subtotals | 1068 | 29 | 2 | 2 | 3 | 1101 |
| Discordant | gold | TP | FN | TP | FN | 4 | | | | | 4 |
| | standard | TP | FN | FN | FN | | | | | | 0 |
| | | FN | TP | FN | TP | 38 | | | | | 38 |
| | | FN | TP | FN | FN | | | | | | 0 |
| | Non-gold | | FP | ND | ND | | | | 132 | | 132 |
| | | | FP | ND | FP | | | | 22 | | 22 |
| | | FP | | ND | ND | | | | 1121 | 1121 | 1121 |
| | | FP | | FP | ND | | | | 8 | | 8 |
| | | | | | Subtotals | 42 | 0 | 0 | 1283 | 1121 | 1325 |
| | | | | | Totals | 1110 | 29 | 2 | 1285 | 1124 | 2426 |

Sensitivity = 99.8%;
PPV = 97.5%

For FFPE MiSeq SVC v.2.1.12 and PGM ITVC v3.6.63335 there were 20,918 MPVD decisions for SNV(s), of which the majority (19,785; 95%) was a false positive in either or both platforms (Table 25).

TABLE 25
Multi-Platform Detection Results for FFPE specimens for SNV(s) in the paired samples.

| | | | | Within | | | | | | | Total |
|---|---|---|---|---|---|---|---|---|---|---|---|
| | | Pre-Multi- | | the Multi- | | | | | | | Multi- |
| | | Platform | | Platform | | Decision of the Multi-Platform Detection | | | | | Platform |
| | | Detection | | Detection | | True | False | False | Not | Failed | Detection |
| | | MiSeq | PGM | MiSeq | PGM | Positive | Positive | Negative | Reported | Testing | Decisions |
| Concordant | gold | TP | TP | TP | TP | 1006 | | | | | 1006 |
| | standard | TP | TP | TP | FN | 13 | | | | 13 | 13 |
| | | TP | TP | FN | TP | 19 | | | | 19 | 19 |
| | | TP | TP | FN | FN | | | 1 | | 1 | 1 |
| | | FN | FN | FN | FN | | | 6 | | | 6 |
| | Non-gold | FP | FP | ND | ND | | | | 52 | 52 | 52 |
| | | FP | FP | ND | FP | | | | 3 | 3 | 3 |
| | | FP | FP | FP | ND | | | | 19 | 19 | 19 |
| | | FP | FP | FP | FP | | 29 | | | | 37 |
| | | | | | Subtotals | 1038 | 37 | 7 | 74 | 107 | 1156 |
| Discordant | gold | TP | FN | TP | FN | 3 | | | | | 3 |
| | standard | TP | FN | FN | FN | | | 2 | | 2 | 2 |
| | | FN | TP | FN | TP | 53 | | | | | 53 |
| | | FN | TP | FN | FN | | | 9 | | 9 | 9 |
| | Non-gold | | FP | ND | ND | | | | 2825 | | 2825 |
| | standard | | FP | ND | FP | | | | 66 | | 66 |
| | | FP | | ND | ND | | | | 15241 | 15241 | 15241 |
| | | FP | | FP | ND | | | | 1563 | | 1563 |
| | | | | | Subtotals | 56 | 0 | 11 | 19695 | 15252 | 19762 |
| | | | | | Totals | 1094 | 37 | 18 | 19769 | 15359 | 20918 |

Sensitivity = 98.3%;
PPV = 96.7%

The majority of these false positives (18,080; 91%) were excluded from further analysis by failing one or more of the MVRT, QUALT, SE and MVAF thresholds or filters. In the MPVD output the remaining 1,705 false positive calls were reduced to 38 as discordant and non-actionable by classification resulting in an assay PPV of 97%. For the 1,039 true positives called by both platforms the vast majority (1,031; 99%) passed all the criteria of MVRT, QUALT, SE and MVAF thresholds or filters. False negatives were only rarely concordant (total=6) and when discordant (TP/FN or FN/TP) (total=67) the paired true positive call failed any of the criteria of MVRT, QUALT, SE and MVAF in only 12 (18%) examples, resulting in a low number of MPVD false negative calls. For concordant true positive calls (total=1,039) both calls failed any of the criteria of MVRT, QUALT, SE and MVAF in only 6 (0.5%), resulting in a combined 24 MPVD false negative calls and an assay sensitivity of 98.3%. The MPVD assay sensitivity and PPV for FFPE MiSeq SVC v.2.1.12 and PGM ITVC v3.6.63335 of 98% and 97%, respectively, is better than the comparable single platform values for FFPE MiSeq SVC v.2.1.12 of 92% and 42% and PGM ITVC v3.6.63335 of 98% and 88%, respectively.

The Paired Samples results for indels showed a substantial improvement within the MPVD. For FF, MiSeq SVC v.2.1.12 and PGM ITVC v3.6.63335 there were 234 MPVD decisions for indels, of which the majority (229 of 234; 98%) were a false positive call in either one or both platforms (Table 26).

TABLE 26
Multi-Platform Detection Results for FF specimens for indels in the Paired Samples.

| | | | | | | | | | | | Total |
|---|---|---|---|---|---|---|---|---|---|---|---|
| | | Pre-Multi- | | Within Multi- | | Decision of Multi-Platform | | | | | Multi- |
| | | Platform | | Platform | | Detection | | | | | Platform |
| | | Detection | | Detection | | True | False | False | Not | Failed | Detection |
| | | MiSeq | PGM | MiSeq | PGM | Pos | Pos | Neg | Reported | Testing | Decisions |
| Concordant | gold | TP | TP | TP | TP | 3 | | | | | 3 |
| | standard | TP | TP | TP | FN | | | | | | |
| | | TP | TP | FN | TP | | | | | | |
| | | TP | TP | FN | FN | | | | | | |
| | | FN | FN | FN | FN | | | | | | |
| | Non-gold | FP | FP | ND | ND | | | | | | |
| | | FP | FP | ND | FP | | | | | | |
| | | FP | FP | FP | ND | | | | | | |
| | | FP | FP | FP | FP | | 1 | | | | 1 |
| | | | | | Subtotals | 3 | 1 | 0 | 0 | 0 | 4 |
| Discordant | gold | TP | FN | TP | FN | 2 | | | | | 3 |
| | standard | TP | FN | FN | FN | | | | | | |
| | | FN | TP | FN | TP | | | | | | |
| | | FN | TP | FN | FN | | | | | | |
| | Non-gold | | FP | ND | ND | | | | 86 | | 86 |
| | | | FP | | FP | | | | 2 | | 2 |
| | | FP | | ND | ND | | | | 134 | | 134 |
| | | FP | | FP | ND | | | | 6 | | 6 |
| | | | | | Subtotals | 2 | 0 | 0 | 228 | 0 | 230 |
| | | | | | Totals | 5 | 1 | 0 | 228 | 0 | 234 |

Sensitivity = 100%;
PPV = 83%

The majority of these false positive calls (220; 97%) were excluded from further analysis by failing one or more of the QUALT, SE and MVAF thresholds or filters. In the MPVD output, the remaining nine false positive calls were reduced to one final false positive call passing the MPVD engine rules, as the other 8 were discordant and non-actionable by classification resulting in an assay PPV of 83%. For the three true positives by both platforms, each passed all the criteria of QUALT, SE and MVAF thresholds and filters. For the two discordant true positives, the true positive call passed all the criteria of QUALT, SE and MVAF filters for the platform identifying the variant. There were no concordant false negatives resulting in a MPVD assay sensitivity for indels of 100%. Assay sensitivity and PPV for FF specimens of 100% and 83%, respectively, is better than the comparable single platform values for FF MiSeq SVC v.2.1.12 of 100% and 42% and PGM ITVC v3.6.63335 of 60% and 50%, respectively.

For FFPE MiSeq SVC v.2.1.12 and PGM ITVC v3.6.63335 there were 419 MPVD decisions for indels, of which the majority (414 of 234; 99%) were a false positive call in either one or both platforms (Table 27).

TABLE 27
Multi-Platform Detection Results for FFPE specimens for indels in the Paired Samples.

| | | | | | | | | | | | Total |
|---|---|---|---|---|---|---|---|---|---|---|---|
| | | Pre-Multi- | | Within Multi- | | Decision of Multi-Platform | | | | | Multi- |
| | | Platform | | Platform | | Detection | | | | | Platform |
| | | Detection | | Detection | | True | False | False | Not | Failed | Detection |
| | | MiSeq | PGM | MiSeq | PGM | Pos | Pos | Neg | Reported | Testing | Decisions |
| Concordant | gold | TP | TP | TP | TP | 3 | | | | | 3 |
| | standard | TP | TP | TP | FN | | | | | | |
| | | TP | TP | FN | TP | | | | | | |
| | | TP | TP | FN | FN | | | | | | |
| | | FN | FN | FN | FN | | | | | | |
| | Non-gold | FP | FP | ND | ND | | | | | | |
| | | FP | FP | ND | FP | | | | | | |
| | | FP | FP | FP | ND | | | | 1 | | 1 |
| | | FP | FP | FP | FP | | 1 | | | | 1 |
| | | | | | Subtotals | 3 | 1 | 0 | 1 | 0 | 5 |
| Discordant | gold | TP | FN | TP | FN | 2 | | | | | 2 |
| | standard | TP | FN | FN | FN | | | | | | |
| | | FN | TP | FN | TP | | | | | | |
| | | FN | TP | FN | FN | | | | | | |
| | Non-gold | | FP | ND | ND | | | | 139 | | 139 |
| | | | FP | ND | FP | | | | 2 | | 2 |
| | | FP | | ND | ND | | | | 258 | | 258 |
| | | FP | | FP | ND | | | | 13 | | 13 |
| | | | | | Subtotals | 2 | 0 | 0 | 412 | 0 | 414 |
| | | | | | Totals | 5 | 1 | 0 | 413 | 0 | 419 |

Sensitivity = 100%;
PPV = 83%

The majority of these false positive calls (397; 98%) were excluded from further analysis by failing one or more of the QUALT, SE and MVAF thresholds or filters. In the MPVD output the remaining 17 false positive calls were reduced to one final false positive call. For the other 16 false positive calls 15 were discordant and non-actionable by classification and one concordant false positive failed the QUALT threshold resulting in an assay PPV of 83%. For the three true positives by both platforms, all of them passed all the criteria of QUALT, SE and MVAF thresholds and filters. For the two discordant true positives, the true positive call passed all the criteria of QUALT, SE and MVAF filters for the platform identifying the variant. There were no concordant false negatives, resulting in a MPVD assay sensitivity for indels of 100%. Assay sensitivity and PPV for FFPE specimens of 100% and 83%, respectively, is better than the comparable single platform values for FFPE MiSeq SVC v.2.1.12 of 100% and 25% and PGM ITVC v3.6.63335 of 60% and 50%, respectively.

For FFPE, MiSeq SVC v.2.1.12 and PGM ITVC v3.6.63335 using the EGFR Samples, there were 9 final variant calls for indels (Table 28).

TABLE 28
MPVD results for EGFR Samples indels.

| | | | | | | | | | | | Total |
|---|---|---|---|---|---|---|---|---|---|---|---|
| | | Pre-Multi- | | Within Multi- | | Decision of Multi-Platform | | | | | Decisions |
| | | Platform | | Platform | | Methods | | | | | of Multi- |
| | | Methods | | Methods | | True | False | False | Not | Failed | Platform |
| | | MiSeq | PGM | MiSeq | PGM | Pos | Pos | Neg | Platform | Testing | Methods |
| Concordant | gold | TP | TP | TP | TP | 0 | 0 | 0 | 0 | 0 | 0 |
| | standard | TP | TP | TP | FN | 0 | 0 | 0 | 0 | 0 | 0 |
| | | TP | TP | FN | TP | 0 | 0 | 0 | 0 | 0 | 0 |
| | | TP | TP | FN | FN | 0 | 0 | 0 | 0 | 0 | 0 |
| | | FN | FN | FN | FN | 0 | 0 | 0 | *1 | 0 | *1 |
| | Non-gold | FP | FP | ND | ND | 0 | 0 | 0 | 0 | 0 | 0 |
| | | FP | FP | ND | FP | 0 | 0 | 0 | 0 | 0 | 0 |
| | | FP | FP | FP | ND | 0 | 0 | 0 | 0 | 0 | 0 |
| | | FP | FP | FP | FP | 0 | 0 | 0 | 0 | 0 | 0 |
| | | | | | Subtotals | 0 | 0 | 0 | 1 | 0 | 0 |
| Discordant | gold | TP | FN | TP | FN | 6 | 0 | 0 | 0 | 0 | 6 |
| | standard | TP | FN | FN | FN | 0 | 0 | 0 | 0 | 0 | 0 |
| | | FN | TP | FN | TP | 0 | 0 | 0 | 0 | 0 | 0 |
| | | FN | TP | FN | FN | 0 | 0 | 0 | 0 | 0 | 0 |
| | Non-gold | | FP | ND | ND | 0 | 0 | 0 | 0 | 0 | 0 |
| | | | FP | ND | FP | 0 | 0 | 0 | 0 | 0 | 0 |
| | | FP | | ND | ND | 0 | 0 | 0 | 0 | 0 | |
| | | FP | | FP | ND | 0 | 0 | 0 | 2 | 0 | 2 |
| | | | | | Subtotals | 6 | 0 | 0 | 2 | 0 | 8 |
| | | | | | Totals | 6 | 0 | 0 | 3 | 0 | 9 |

*Intel not detected in either platform and not present in wither BAM file resulting in update to the gold standard.
Sensitivity 100%;
PPV = 100%

Six were discordant true positives for which the true positive call passed all the criteria of QUALT, SE and MVAF filters for the platform identifying the variant. One concordant false negative variant resulted in an update to the gold standard and not reported (NR) classification within the MPVD. The remaining two MPVD decisions were non-gold standard discordant false positive calls that resulted in an additional 2 not reported (NR) classifications within the MPVD. 100% assay sensitivity and PPV for the FFPE EGFR Samples is better than the comparable single platform values for FFPE MiSeq SVC v.2.1.12 of 100% and 86%, and no results for PGM ITVC v3.6.63335.

### Summary of Variant Call Results

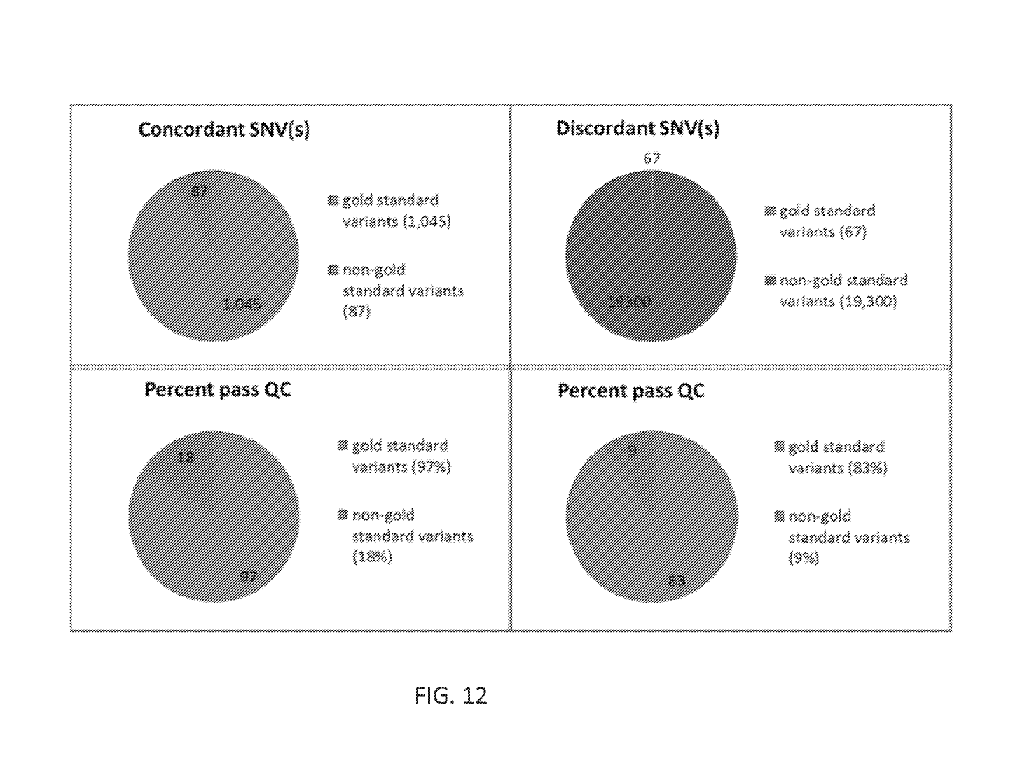

There were several high level results that underscore the strength of the MPVD, and parallel sequencing on both the MiSeq and PGM, with an integrated approach to actionable or gold standard variants. FIG. 12 is a summary of results using the 20-gene validation testing, which exemplifies embodiments of the disclosed methods and systems. As shown in FIG. 12, first, concordant SNV(s) were dominated by gold standard variants while discordant variants were dominated by non-gold standard variants. For FFPE, prior to the MPVD there were 1,132 concordant calls of which 1,045 (92%) were gold standard variants (TP/TP or FN/FN) and 87 (8%) were non-gold standard variants (FP/FP). This compares to the 19,367 discordant variants of which 67 (0.3%) were gold standard variants (TP/FN or FN/TP) and 19,300 (97.3%) were non-gold standard variants (FP/no variant in the other platform).

As shown in FIG. 12, a second result that underscored the strength of the methods and systems was the percentage of SNV(s) within each of these high level groupings that passed all the criteria of the MVRT, QUALT, SE and MVAF thresholds and filters. Of the 1,045 concordant gold standard variants, 1,016 (97%) passed MVRT, QUALT, SE and MVAF, while only 16 (18%) of the 87 concordant non-gold standard variants passed. In the discordant variant group, 56 (83%) of the 67 discordant gold standard variants passed MVRT, QUALT, SE and MVAF, while only 1,809 (9%) of the 19,300 discordant non-gold standard variants passed.

The combined result of these two high level observations is that true positives are generally high quality reads detected on both platforms, while false positives are generally low quality and detected on only one platform. This is supported by the fact that there were 1,038 concordant true positive calls versus 67 discordant TP/FN or FN/TP calls for SNV(s). This compares to the 87 concordant false positive calls versus the 19,300 discordant false positive calls whereby only one of the two platforms, MiSeq or PGM, detected a false positive. Additionally, there were only six concordant false negative calls for which both calls would have resulted in a failed testing classification by the variant detection methods and systems in a clinical setting due to less reads in the BAM file than the MVAR.

The strength of the methods and systems of the invention versus a single sequencing platform for indels was highlighted by a marked improvement in PPV with limited impact for sensitivity due to the excellent detection of this variant type by MiSeq SVC as a single sequencing platform. Optionally, manual review of discordant indels with the BAM pileups, as set forth above, can be done. Defining a specific value for assay sensitivity and PPV for indels for 20 Gene NGS1 had some limitations given the limited number of indels in both the Paired Samples and the EGFR Samples. In regard to indel analysis for a single sequencing platform the MiSeq FF maintained greater than 95% sensitivity in the Pooled Sample ranging from a VAF of 2.9% to 10.8%, while for MiSeq FFPE sensitivity of 95% in the Pooled Sample was limited to the one variant with the highest VAF of 3.6%. The MVAF of 2.9% for MiSeq FF and 3.6% for MiSeq FFPE defined in the Pooled Sample is supported by 100% detection of all variants above this VAF in the Paired Samples and the EGFR Samples. For MiSeq FF and FFPE in the Paired Samples all gold standard indels were detected where the VAF ranged from 15% to 48% and 21% to 47%, respectively. Additionally, for MiSeq FFPE in the EGFR Samples there was 100% sensitivity with the corresponding VAF(s) ranging from 4.8% to 70%. It is important to note that the most common activating indels in EGFR, which are deletions in exon 19 centered around four amino acids at codon positions 747-750, and along with the L858R missense mutation, constitute 90% of all EGFR activating mutations, were well represented in the EGFR Samples. Primers are being designed that target EGFR exon 19 deletions in the ITAS (PGM) enrichment process, which will give us further confidence in our detection of actionable variants for this gene.

Assay sensitivity for indels within the MPVD for both the Paired Samples and the EGFR Samples was 100% and this value will be our final 20 Gene NGS1 result for both FF and FFPE at a MVAF or 2.9% and 3.6%, respectively. Assay PPV for indels, within the MPVD for FF, was limited to evaluation of the Paired Samples with 100% sensitivity and 83% PPV at a MVAF of 2.9%. Assay PPV for indels for FFPE within the MPVD varied from 83% in the Paired Samples to 100% for the Lung EGFR Samples for FFPE, but with the latter limited to only analysis of the EGFR gene. For purposes of this validation for FFPE, we have taken an average of the assay PPV for Paired Samples and EGFR of 91% as our validated value.

The final values for the MPVD for sensitivity and PPV are summarized in Table 29.

TABLE 29
Assay sensitivity and PPV using Variant Calling Methods

| | FF | | | | FFPE | | | |
|---|---|---|---|---|---|---|---|---|
| | SNV(s) | | Indels | | SNV(s) | | Indels | |
| | Percent | VAF | Percent | VAF | Percent | VAF | Percent | VAF |
| Assay Sensitivity | 99.8% | 2.87% | 100.0% | 2.90% | 98.3% | 3.56% | 100.0% | 3.60% |
| Assay PPV | 97.5% | | 91.0% | | 96.7% | | 91.0% | |

1. A method for detecting the presence of at least one specific allelic variant in a biological sample, comprising:
(a) receiving first sequencing data produced by sequencing a first aliquot of nucleic acids from the biological sample using a first sequencing platform;
(b) receiving second sequencing data produced by sequencing a second aliquot of nucleic acids from the biological sample using a second sequencing platform, the first sequencing platform being the same as or differing from the second sequencing platform;
wherein the first sequencing data and second sequencing data comprise the nucleotide sequences of a multiplicity of sequencing reads including a multiplicity of allelic variants;
(c) selecting from the multiplicity of allelic variants in the first sequencing data and second sequencing data at least one specific allelic variant for analysis;
(d) detecting the presence of the specific allelic variant in the biological sample if either:
(i) a first analysis of the first sequencing data relating to the specific allelic variant passes at least one filter selected from the group consisting of absence of a first platform-dependent systematic error, a first platform-sample-target-dependent minimum variant read threshold and a first platform-sample-target-dependent minimum variant allelic frequency, or
(ii) a second analysis of the second sequencing data relating to the specific allelic variant passes at least one filter selected from the group consisting of absence of a second platform-dependent systematic error, a second platform-sample-target-dependent minimum variant read threshold and a second platform-sample-target-dependent minimum variant allelic frequency.

2. The method of claim 1, wherein the first sequencing data is based on sequencing nucleic acids amplified from the biological sample using the first sequencing platform, the second sequencing platform, or both.

3. (canceled)

4. The method of claim 1, wherein the specific allelic variant is selected from the group consisting of a subset of the multiplicity of variants comprising known therapeutically actionable variants, a subset of the multiplicity of variants which does not include at least one known therapeutically non-actionable variant, from a subset of possible variants which comprises known diagnostically informative variants, from a predefined list of variants which does not include at least one known diagnostically non-informative variant, a subset of possible variants which comprises known prognostically informative variants, and a subset of possible variants which does not include at least one known prognostically non-informative variant, and combinations thereof.

5. (canceled)

6. (canceled)

7. (canceled)

8. (canceled)

9. (canceled)

10. The method of claim 1, wherein the at least one filter in one or both of the first and second analyses is selected from the group consisting of a platform-sample-target-dependent minimum variant read threshold or a platform-sample-target-dependent minimum variant allele frequency, and combinations thereof.

11. (canceled)

12. The method of claim 10, wherein in one or both of the platform-sample-target-dependent minimum variant read threshold and the platform-sample-target-dependent minimum variant allele frequency are empirically determined by sequencing at least one control nucleic acid sample or wherein one or both of the threshold and the frequency are known from sequencing at least one control nucleic acid sample, and combinations thereof.

13. (canceled)

14. (canceled)

15. (canceled)

16. (canceled)

17. (canceled)

18. (canceled)

19. The method of claim 12, wherein the control nucleic acid sample comprises the specific allelic variant.

20. The method of claim 12, wherein the minimum variant allele frequency is selected from a range of about less than 4.0% to about less than 2.0%.

21. (canceled)

22. (canceled)

23. (canceled)

24. (canceled)

25. (canceled)

26. (canceled)

27. (canceled)

28. (canceled)

29. (canceled)

30. The method of claim 1 wherein detecting the presence of the specific allelic variant in step (d) further requires that either:
(i) the first analysis of the first sequencing data relating to the specific allelic variant passes at least two filters selected from the group consisting of absence of a first platform-dependent systematic error, a first platform-sample-target-dependent minimum variant read threshold, and a first platform-sample-target-dependent minimum variant allelic frequency, or
(ii) the second analysis of the second sequencing data relating to the specific allelic variant passes at least two filters selected from the group consisting of absence of a second platform-dependent systematic error, a second platform-sample-target-dependent minimum variant read threshold, and a second platform-sample-target-dependent minimum variant allelic frequency.

31. (canceled)

32. (canceled)

33. A method comprising:
(a) receiving first sequencing data indicative of a presence or absence of a specific allelic variant in a biological sample based on results from a first sequencing process performed on a first sequencing platform, the first sequencing data comprising nucleotide sequences of a multiplicity of sequencing reads including a first multiplicity of allelic variants;
(b) receiving second sequencing data indicative of a presence or absence of the specific allelic variant in the biological sample based on results from a second sequencing process performed on a second sequencing platform, the second sequencing data comprising nucleotide sequences of a multiplicity of sequencing reads including a second multiplicity of allelic variants;
(c) determining at least one first filter value based on base-pair level characteristics of a biological standard comprising the specific allelic variant detected by the first sequencing platform, wherein the at least one first filter value is selected from the group consisting of:
a first platform-sample-target-dependent minimum variant reads threshold,
a first platform-sample-target-dependent minimum variant allelic frequency, and
a first sample-dependent set of systematic errors;
(d) conducting a first comparison of the at least one first filter value to the first sequencing data to determine if the data indicative of the presence or absence of the specific allelic variant passes the first filter value;
(e) determining at least one second filter value based on base-pair level characteristics of the biological standard comprising the specific allelic variant detected by the second sequencing platform, wherein the at least one second filter value is selected from the group consisting of:
a second platform-sample-target-dependent minimum variant reads threshold,
a second platform-sample-target-dependent minimum variant allelic frequency, and
a set sample-dependent of second systematic errors;
(f) conducting a second comparison of the at least one second filter value to the second sequencing data to determine if the data indicative of the presence or absence of the specific allelic variant passes the second filter value; and
(g) detecting the presence or absence of the specific allelic variant in the biological sample based on the results of the first comparison and the second comparison.

34. The method of claim 33, wherein one or both of the first and the second sequencing data indicative of the presence or absence of a specific allelic variant in the biological sample is based on sequencing nucleic acids amplified from the biological sample using the first sequencing platform.

35. (canceled)

36. The method of claim 33, wherein the specific allelic variant is selected from the group consisting of one or more subsets of the multiplicity of variants comprising known therapeutically actionable variants, the multiplicity of variants which does not include at least one known therapeutically non-actionable variant, possible variants which comprises known diagnostically informative variants, a predefined list of variants which does not include at least one known diagnostically non-informative variant, possible variants which comprises known prognostically informative variants, possible variants which does not include at least one known prognostically non-informative variant.

37. (canceled)

38. (canceled)

39. (canceled)

40. (canceled)

41. (canceled)

42. The method of claim 33, wherein the at least one first filter value in the first comparison is the first platform-sample-target-dependent minimum variant read threshold, or wherein the at least one first filter value in the second comparison is the second platform-sample-target-dependent minimum variant read threshold.

43. (canceled)

44. (canceled)

45. (canceled)

46. (canceled)

47. The method of claim 33, wherein the at least one first filter value in the first comparison is the first platform-sample-target-dependent minimum variant allele frequency, or wherein the at least one filter value in the second comparison is the second platform-sample-target-dependent minimum variant allele frequency, or both.

48. (canceled)

49. The method of claim 33, wherein at least one of the first and the second platform-sample-target-dependent minimum variant allele frequency is (i) empirically determined by sequencing at least one control nucleic acid sample or (ii) is known from sequencing at least one control nucleic acid sample, or both.

50. (canceled)

51. (canceled)

52. The method of claim 33, wherein at least one of the first and the second platform-sample-target-dependent minimum variant allele frequency is selected from a range of about less than 4.0% to about less than 2.0%.

53. (canceled)

54. (canceled)

55. (canceled)

56. (canceled)

57. (canceled)

58. (canceled)

59. (canceled)

60. (canceled)

61. (canceled)

62. The method of claim 33 wherein the detecting the presence of the specific allelic variant further requires that either:
(i) the first comparison of the first sequencing data relating to the specific allelic variant passes at least two filters values selected from the group consisting of the first platform-sample-target-dependent minimum variant reads threshold, the first platform-sample-target-dependent minimum variant allelic frequency, and absence of the first sample-dependent set of systematic errors, or
(ii) the second comparison of the second sequencing data relating to the specific allelic variant passes at least two filters values selected from the group consisting of the second platform-sample-target-dependent minimum variant reads threshold, the second platform-sample-target-dependent minimum variant allelic frequency, and absence of the second sample-dependent set of systematic errors.

63. (canceled)

64. (canceled)

65. The method of claim 33, wherein the conducting the first comparison includes:
forming a first subset of sequencing data including only those values from the first sequencing data that do not exhibit the presence of the first sample-dependent set of systematic errors; and
conducting a further comparison of the first subset of sequencing data to at least one of the first platform-sample-target-dependent minimum variant reads threshold and the first platform-sample-target-dependent minimum variant allelic frequency to determine if the data indicative of the presence or absence of the specific allelic variant in the first subset passes the at least one of the first platform-sample-target-dependent minimum variant reads threshold and the first platform-sample-target-dependent minimum variant allelic frequency, and wherein:
the conducting the second comparison includes:
forming a second subset of sequencing data including only those values from the second sequencing data that do not exhibit the presence of the second sample-dependent set of systematic errors; and
conducting a further comparison of the second subset of sequencing data to at least one of the second platform-sample-target-dependent minimum variant reads threshold and the second platform-sample-target-dependent minimum variant allelic frequency to determine if the data indicative of the presence or absence of the specific allelic variant in the second subset passes the at least one of the second platform-sample-target-dependent minimum variant reads threshold and the second platform-sample-target-dependent minimum variant allelic frequency.

66. (canceled)

67. (canceled)

68. (canceled)

69. A system comprising:
a first sequencing platform apparatus;
a second sequencing platform apparatus;
a multi-platform variant detection system, comprising:
a first interface for receiving first sequencing data indicative of a presence or absence of a specific allelic variant in a biological sample based on results from a first sequencing process performed on the first sequencing platform;
a second interface for receiving second sequencing data indicative of a presence or absence of a specific allelic variant in the biological sample based on results from a second sequencing process performed on the second sequencing platform;
a computer-readable memory comprising at least one first filter value based on base-pair level characteristics of a biological standard comprising the specific allelic variant detected by the first sequencing platform, wherein the first filter value is selected from the group consisting of:
a first platform-sample-target-dependent minimum variant reads threshold,
a first platform-sample-target-dependent minimum variant allelic frequency, and
a first sample-dependent set of systematic errors,
the computer-readable memory comprising at least one second filter value based on base-pair level characteristics of the biological standard comprising the specific allelic variant detected by the second sequencing platform, wherein the second filter value is selected from the group consisting of:
a second platform-sample-target-dependent minimum variant reads threshold,
a second platform-sample-target-dependent minimum variant allelic frequency filter, and
a second sample-dependent set of systematic errors; and
the computer-readable memory comprising instructions that when executed cause the multi-platform variant detection system to:
conduct a first comparison of the first at least one filter value to the first sequencing data to determine if the data indicative of the presence or absence of the specific allelic variant passes the at least one first filter value;
conduct a second comparison of the second at least one filter value to the second sequencing data to determine if the data indicative of the presence or absence of the specific allelic variant passes the second at least one filter value; and
detect the presence or absence of the specific allelic variant in the biological sample based on the results of the first comparison and the second comparison.

70. The method of claim 69, wherein the first sequencing data indicative of the presence or absence of a specific allelic variant in the biological sample is based on sequencing nucleic acids amplified from the biological sample using the first sequencing platform, or wherein the second sequencing data indicative of the presence or absence of a specific allelic variant in the biological sample is based on sequencing nucleic acids amplified from the biological sample using the second sequencing platform, or both.

71. (canceled)

72. The system of claim 69, wherein the specific allelic variant is selected from the group consisting of a subset of the multiplicity of variants comprising known therapeutically actionable variants, a subset of the multiplicity of variants which does not include at least one known therapeutically non-actionable variant, from a subset of possible variants which comprises known diagnostically informative variants, from a predefined list of variants which does not include at least one known diagnostically non-informative variant, a subset of possible variants which comprises known prognostically informative variants, and a subset of possible variants which does not include at least one known prognostically non-informative variant, and combinations thereof.

73.-105. (canceled)
